SEQUENCE LISTING (192 sequences)

SEQ ID NO: 1 - protein, 89 aa, Homo sapiens [sequence omitted]

SEQ ID NO: 2 - protein, 67 aa, Homo sapiens
  PEPTIDE (1)..(37): Human ProIAPP
TPIESHQVEKRKCNTATCATQRLANFLVHSCNNFGAILSSTNVGSNTYGKRNAVEVLKREPLNYLPL

SEQ ID NO: 3 - protein, 37 aa, Homo sapiens [sequence omitted]

SEQ ID NO: 4 - protein, 37 aa, Felis catus
  PEPTIDE (1)..(37): Cat IAPP (Amylin)
KCNTATCATQRLANFLIRSSNNLGAILSPTNVGSNTY

SEQ ID NO: 5 - protein, 37 aa, Macaca fascicularis
  PEPTIDE (1)..(37): Macaque  IAPP (Amylin)
KCNTATCATQRLANFLVRSSNNFGTILSSTNVGSDTY

SEQ ID NO: 6 - protein, 37 aa, Rattus norvegicus
  PEPTIDE (1)..(37): Rat / Mouse IAPP (Amylin) 1-37
KCNTATCATQRLANFLVRSSNNLGPVLPPTNVGSNTY

SEQ ID NO: 7 - protein, 37 aa, Cavia porcellus
  PEPTIDE (1)..(37): Guinea pig IAPP (Amylin)
KCNTATCATQRLTNFLVRSSHNLGAALLPTDVGSNTY

SEQ ID NO: 8 - protein, 9 aa, Homo sapiens
  PEPTIDE (1)..(9): IAPP 1-9
KCNTATCAT

SEQ ID NO: 9 - protein, 13 aa, Homo sapiens
  PEPTIDE (1)..(13): IAPP 1-13
KCNTATCATQRLA

SEQ ID NO: 10 - protein, 16 aa, Homo sapiens
  PEPTIDE (1)..(16): IAPP 1-16
KCNTATCATQRLANFL

SEQ ID NO: 11 - protein, 6 aa, Homo sapiens
  PEPTIDE (1)..(6): IAPP 2-7
CNTATC

SEQ ID NO: 12 - protein, 12 aa, Homo sapiens
  PEPTIDE (1)..(12): IAPP 2-13
CNTATCATQRLA

SEQ ID NO: 13 - protein, 14 aa, Homo sapiens
  PEPTIDE (1)..(14): IAPP 2-15
CNTATCATQRLANF

SEQ ID NO: 14 - protein, 10 aa, Homo sapiens
  PEPTIDE (1)..(10): IAPP 11-20
RLANFLVHSS

SEQ ID NO: 15 - protein, 10 aa, Homo sapiens
  PEPTIDE (1)..(10): IAPP 20-29
SNNFGAILSS

SEQ ID NO: 16 - protein, 21 aa, Homo sapiens
  PEPTIDE (1)..(21): IAPP 10-30
QRLANFLVHSSNNFGAILSST

SEQ ID NO: 17 - protein, 27 aa, Homo sapiens
  PEPTIDE (1)..(27): IAPP 11-37
RLANFLVHSSNNFGAILSSTNVGSNTY

SEQ ID NO: 18 - protein, 23 aa, Homo sapiens
  PEPTIDE (1)..(23): IAPP 15-37
FLVHSSNNFGAILSSTNVGSNTY

SEQ ID NO: 19 - protein, 13 aa, Homo sapiens
  PEPTIDE (1)..(13): IAPP 18-30
HSSNNFGAILSST

SEQ ID NO: 20 - protein, 16 aa, Homo sapiens
  PEPTIDE (1)..(16): IAPP 18-33
HSSNNFGAILSSTNVG

SEQ ID NO: 21 - protein, 18 aa, Homo sapiens
  PEPTIDE (1)..(18): IAPP 18-35
HSSNNFGAILSSTNVGSN

SEQ ID NO: 22 - protein, 20 aa, Homo sapiens
  PEPTIDE (1)..(20): IAPP 18-37
HSSNNFGAILSSTNVGSNTY

SEQ ID NO: 23 - protein, 18 aa, Homo sapiens
  PEPTIDE (1)..(18): IAPP 20-37
SNNFGAILSSTNVGSNTY

SEQ ID NO: 24 - protein, 15 aa, Homo sapiens
  PEPTIDE (1)..(15): IAPP 23-37
FGAILSSTNVGSNTY

SEQ ID NO: 25 - protein, 13 aa, Homo sapiens
  PEPTIDE (1)..(13): IAPP 25-37
AILSSTNVGSNTY

SEQ ID NO: 26 - protein, 8 aa, Homo sapiens
  PEPTIDE (1)..(8): IAPP 30-37
TNVGSNTY

SEQ ID NO: 27 - protein, 10 aa, Homo sapiens
  PEPTIDE (1)..(10): IAPP -8 - 2
ESHQVEKRKC

SEQ ID NO: 28 - protein, 10 aa, Homo sapiens
  PEPTIDE (1)..(10): IAPP -7 - 3
SHQVEKRKCN

SEQ ID NO: 29 - protein, 10 aa, Homo sapiens
  PEPTIDE (1)..(10): IAPP -6 - 4
HQVEKRKCNT

SEQ ID NO: 30 - protein, 10 aa, Homo sapiens
  PEPTIDE (1)..(10): IAPP -5 - 5
QVEKRKCNTA

SEQ ID NO: 31 - protein, 10 aa, Homo sapiens
  PEPTIDE (1)..(10): IAPP -4 - 6
VEKRKCNTAT

SEQ ID NO: 32 - protein, 10 aa, Homo sapiens
  PEPTIDE (1)..(10): IAPP -3 - 7
EKRKCNTATC

SEQ ID NO: 33 - protein, 10 aa, Homo sapiens
  PEPTIDE (1)..(10): IAPP -2 - 8
KRKCNTATCA

SEQ ID NO: 34 - protein, 10 aa, Homo sapiens
  PEPTIDE (1)..(10): IAPP -1 - 9
RKCNTATCAT

SEQ ID NO: 35 - protein, 10 aa, Homo sapiens
  PEPTIDE (1)..(10): IAPP 1 - 10
KCNTATCATQ

SEQ ID NO: 36 - protein, 10 aa, Homo sapiens
  PEPTIDE (1)..(10): IAPP 2 - 11
CNTATCATQR

SEQ ID NO: 37 - protein, 10 aa, Homo sapiens
  PEPTIDE (1)..(10): IAPP 3 - 12
NTATCATQRL

SEQ ID NO: 38 - protein, 10 aa, Homo sapiens
  PEPTIDE (1)..(10): IAPP 4 - 13
TATCATQRLA

SEQ ID NO: 39 - protein, 10 aa, Homo sapiens
  PEPTIDE (1)..(10): IAPP 5 - 14
ATCATQRLAN

SEQ ID NO: 40 - protein, 10 aa, Homo sapiens
  PEPTIDE (1)..(10): IAPP 6 - 15
TCATQRLANF

SEQ ID NO: 41 - protein, 10 aa, Homo sapiens
  PEPTIDE (1)..(10): IAPP 7 - 16
CATQRLANFL

SEQ ID NO: 42 - protein, 10 aa, Homo sapiens
  PEPTIDE (1)..(10): IAPP 8 - 17
ATQRLANFLV

SEQ ID NO: 43 - protein, 10 aa, Homo sapiens
  PEPTIDE (1)..(10): IAPP 9 - 18
TQRLANFLVH

SEQ ID NO: 44 - protein, 10 aa, Homo sapiens
  PEPTIDE (1)..(10): IAPP 10 - 19
QRLANFLVHS

SEQ ID NO: 45 - protein, 10 aa, Homo sapiens
  PEPTIDE (1)..(10): IAPP 11 - 20
RLANFLVHSS

SEQ ID NO: 46 - protein, 10 aa, Homo sapiens
  PEPTIDE (1)..(10): IAPP 12 - 21
LANFLVHSSN

SEQ ID NO: 47 - protein, 10 aa, Homo sapiens
  PEPTIDE (1)..(10): IAPP 13 - 22
ANFLVHSSNN

SEQ ID NO: 48 - protein, 10 aa, Homo sapiens
  PEPTIDE (1)..(10): IAPP 14 - 23
NFLVHSSNNF

SEQ ID NO: 49 - protein, 10 aa, Homo sapiens
  PEPTIDE (1)..(10): IAPP 15 - 24
FLVHSSNNFG

SEQ ID NO: 50 - protein, 10 aa, Homo sapiens
  PEPTIDE (1)..(10): Amylin 16 - 25
  PEPTIDE (1)..(10): IAPP 16 - 25
LVHSSNNFGA

SEQ ID NO: 51 - protein, 10 aa, Homo sapiens
  PEPTIDE (1)..(10): IAPP 17 - 26
VHSSNNFGAI

SEQ ID NO: 52 - protein, 10 aa, Homo sapiens
  PEPTIDE (1)..(10): IAPP 18 - 27
HSSNNFGAIL

SEQ ID NO: 53 - protein, 10 aa, Homo sapiens
  PEPTIDE (1)..(10): IAPP 19 - 28
SSNNFGAILS

SEQ ID NO: 54 - protein, 10 aa, Homo sapiens
  PEPTIDE (1)..(10): IAPP 20 - 29
SNNFGAILSS

SEQ ID NO: 55 - protein, 10 aa, Homo sapiens
  PEPTIDE (1)..(10): IAPP 21 - 30
NNFGAILSST

SEQ ID NO: 56 - protein, 10 aa, Homo sapiens
  PEPTIDE (1)..(10): IAPP 22- 31
NFGAILSSTN

SEQ ID NO: 57 - protein, 10 aa, Homo sapiens
  PEPTIDE (1)..(10): IAPP 23 - 32
FGAILSSTNV

SEQ ID NO: 58 - protein, 10 aa, Homo sapiens
  PEPTIDE (1)..(10): IAPP 24 - 33
GAILSSTNVG

SEQ ID NO: 59 - protein, 10 aa, Homo sapiens
  PEPTIDE (1)..(10): IAPP 25 - 34
AILSSTNVGS

SEQ ID NO: 60 - protein, 10 aa, Homo sapiens
  PEPTIDE (1)..(10): IAPP 26 - 35
ILSSTNVGSN

SEQ ID NO: 61 - protein, 10 aa, Homo sapiens
  PEPTIDE (1)..(10): IAPP 27 - 36
LSSTNVGSNT

SEQ ID NO: 62 - protein, 10 aa, Homo sapiens
  PEPTIDE (1)..(10): IAPP 28 - 37
SSTNVGSNTY

SEQ ID NO: 63 - protein, 10 aa, Homo sapiens
  PEPTIDE (1)..(10): IAPP 29 - 38
STNVGSNTYG

SEQ ID NO: 64 - protein, 10 aa, Homo sapiens
  PEPTIDE (1)..(10): IAPP 30 - 39
TNVGSNTYGK

SEQ ID NO: 65 - protein, 10 aa, Homo sapiens
  PEPTIDE (1)..(10): IAPP 31 - 40
NVGSNTYGKR

SEQ ID NO: 66 - protein, 10 aa, Homo sapiens
  PEPTIDE (1)..(10): IAPP 32 - 41
VGSNTYGKRN

SEQ ID NO: 67 - protein, 10 aa, Homo sapiens
  PEPTIDE (1)..(10): IAPP 33 - 42
GSNTYGKRNA

SEQ ID NO: 68 - protein, 10 aa, Homo sapiens
  PEPTIDE (1)..(10): IAPP 34 - 43
SNTYGKRNAV

SEQ ID NO: 69 - protein, 10 aa, Homo sapiens
  PEPTIDE (1)..(10): IAPP 35 - 44
NTYGKRNAVE

SEQ ID NO: 70 - protein, 6 aa, Artifical sequence
  Flexible Hinge Spacer
  PEPTIDE (1)..(6): Flexible Hinge Spacer
  SITE (3)..(3): Xaa is any amino acid, preferably Asp
  SITE (5)..(5): Xaa is any amino acid, preferably Asp
PPXPXP

SEQ ID NO: 71 - protein, 4 aa, synthetic peptide
  SITE (1)..(1): epsilon-K
  PEPTIDE (1)..(4): epsilon-K-KKK as a spacer
KKKK

SEQ ID NO: 72 - protein, 4 aa, synthetic peptide
  PEPTIDE (1)..(4): epsilon-K-KKK as a spacer
  SITE (4)..(4): epsilon-K
KKKK

SEQ ID NO: 73 - protein, 17 aa, Clostridium tetani
  PEPTIDE (1)..(17): Clostridium tetani 1 Th
KKQYIKANSKFIGITEL

SEQ ID NO: 74 - protein, 15 aa, Measles virus
  PEPTIDE (1)..(15): MvF1 Th
LSEIKGVIVHRLEGV

SEQ ID NO: 75 - protein, 24 aa, Bordetella pertussis
  PEPTIDE (1)..(24): Bordetella pertussis Th
GAYARCPNGTRALTVAELRGNAEL

SEQ ID NO: 76 - protein, 17 aa, Clostridium tetani
  PEPTIDE (1)..(17): Clostridium tetani 2 Th
WVRDIIDDFTNESSQKT

SEQ ID NO: 77 - protein, 23 aa, diphtheria bacilli
  PEPTIDE (1)..(23): Diphtheria Th
DSETADNLEKTVAALSILPGHGC

SEQ ID NO: 78 - protein, 21 aa, Plasmodium falciparum
  PEPTIDE (1)..(21): Plasmodium falciparum Th
DHEKKHAKMEKASSVFNVVNS

SEQ ID NO: 79 - protein, 17 aa, Schistosoma mansoni
  PEPTIDE (1)..(17): Schistosoma mansoni Th
KWFKTNAPNGVDEKHRH

SEQ ID NO: 80 - protein, 25 aa, Cholera Toxin
  PEPTIDE (1)..(25): Cholera Toxin Th
ALNIWDRFDVFCTLGATTGYLKGNS

SEQ ID NO: 81 - protein, 15 aa, Measles virus
  PEPTIDE (1)..(15): MvF 2 Th
ISEIKGVIVHKIEGI

SEQ ID NO: 82 - protein, 22 aa, Measles virus
  PEPTIDE (1)..(22): KKKMvF3 Th
KKKISISEIKGVIVHKIEGILF

SEQ ID NO: 83 - protein, 22 aa, Measles virus
  PEPTIDE (1)..(22): KKKMvF3 Th
KKKISITEIRTVIVTRIETILF

SEQ ID NO: 84 - protein, 22 aa, Measles virus
  PEPTIDE (1)..(22): KKKMvF 3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
KKKISIXEIXXVIVXXIEXILF

SEQ ID NO: 85 - protein, 18 aa, Hepatitis B virus
  PEPTIDE (1)..(18): HBsAg1 Th
KKKLFLLTKLLTLPQSLD

SEQ ID NO: 86 - protein, 18 aa, Hepatitis B virus
  PEPTIDE (1)..(18): HBsAg1 Th
RRRIKIITRIITIPLSIR

SEQ ID NO: 87 - protein, 18 aa, Hepatitis B virus
  PEPTIDE (1)..(18): HBsAg1 Th
KKKVRVVTKVVTVPISVD

SEQ ID NO: 88 - protein, 18 aa, Hepatitis B virus
  PEPTIDE (1)..(18): HBsAg1 Th
KKKFFFFTKFFTFPVSFD

SEQ ID NO: 89 - protein, 18 aa, Hepatitis B virus
  PEPTIDE (1)..(18): HBsAg1 Th
KKKLFLLTKLLTLPFSLD

SEQ ID NO: 90 - protein, 18 aa, Hepatitis B virus
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
XXXXXXXTXXXTXPXSXX

SEQ ID NO: 91 - protein, 19 aa, Measles virus
  PEPTIDE (1)..(19): MvF4 Th
ISISEIKGVIVHKIETILF

SEQ ID NO: 92 - protein, 19 aa, Measles virus
  PEPTIDE (1)..(19): MvF4 Th
ISITEIRTVIVTRIETILF

SEQ ID NO: 93 - protein, 19 aa, Measles virus
  PEPTIDE (1)..(19): MvF 4 Th
  SITE (4)..(4): S or T
  SITE (7)..(7): K or R
  SITE (8)..(8): G or T
  SITE (12)..(12): H or T
  SITE (13)..(13): K or R
ISIXEIXXVIVXXIETILF

SEQ ID NO: 94 - protein, 18 aa, Hepatitis B virus
  PEPTIDE (1)..(18): HBsAg2 Th
KKKIITITRIITIPQSLD

SEQ ID NO: 95 - protein, 18 aa, Hepatitis B virus
  PEPTIDE (1)..(18): HBsAg2 Th
KKKFFLLTRILTIITTID

SEQ ID NO: 96 - protein, 18 aa, Hepatitis B virus
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
KKKXXXXTRIXTIXXXXD

SEQ ID NO: 97 - protein, 19 aa, Measles virus
  PEPTIDE (1)..(19): MvF 5 Th
ISITEIKGVIVHRIETILF

SEQ ID NO: 98 - protein, 18 aa, Hepatitis B virus
  PEPTIDE (1)..(18): HBsAg 3 Th
KKKIITITRIITIITTID

SEQ ID NO: 99 - protein, 11 aa, Influenza virus
  PEPTIDE (1)..(11): Influenza Matrix protein 1 _1 Th
  PEPTIDE (1)..(11): Influenza Matrix protein 1_1 Th
FVFTLTVPSER

SEQ ID NO: 100 - protein, 15 aa, Influenza virus
  PEPTIDE (1)..(15): Influenza Matrix protein 1_2 Th
SGPLKAEIAQRLEDV

SEQ ID NO: 101 - protein, 9 aa, Influenza virus
  PEPTIDE (1)..(9): Influenza Non-structural protein 1 Th
DRLRRDQKS

SEQ ID NO: 102 - protein, 19 aa, Epstein-Barr virus
  PEPTIDE (1)..(19): EBV BHRF1 Th
AGLTLSLLVICSYLFISRG

SEQ ID NO: 103 - protein, 15 aa, Clostridium tetani
  PEPTIDE (1)..(15): Clostridium tetani TT1 Th
QYIKANSKFIGITEL

SEQ ID NO: 104 - protein, 20 aa, Epstein-Barr virus
  PEPTIDE (1)..(20): EBV EBNA-1 Th
PGPLRESIVCYFMVFLQTHI

SEQ ID NO: 105 - protein, 21 aa, Clostridium tetani
  PEPTIDE (1)..(21): Clostridium tetani  TT2 Th
FNNFTVSFWLRVPKVSASHLE

SEQ ID NO: 106 - protein, 16 aa, Clostridium tetani
  PEPTIDE (1)..(16): Clostridium tetani TT3 Th
KFIIKRYTPNNEIDSF

SEQ ID NO: 107 - protein, 16 aa, Clostridium tetani
  PEPTIDE (1)..(16): Clostridium tetani  TT4 Th
VSIDKFRIFCKALNPK

SEQ ID NO: 108 - protein, 18 aa, Epstein-Barr virus
  PEPTIDE (1)..(18): EBV CP Th
VPGLYSPCRAFFNKEELL

SEQ ID NO: 109 - protein, 14 aa, Human cytomegalovirus
  PEPTIDE (1)..(14): HCMV IE1 Th
DKREMWMACIKELH

SEQ ID NO: 110 - protein, 15 aa, Epstein-Barr virus
  PEPTIDE (1)..(15): EBV GP340 Th
TGHGARTSTEPTTDY

SEQ ID NO: 111 - protein, 13 aa, Epstein-Barr virus
  PEPTIDE (1)..(13): EBV BPLF1 Th
KELKRQYEKKLRQ

SEQ ID NO: 112 - protein, 11 aa, Epstein-Barr virus
  PEPTIDE (1)..(11): EBV EBNA-2 Th
TVFYNIPPMPL

SEQ ID NO: 113 - protein, 32 aa, Artificial Sequence
  Synthetic peptide
  PEPTIDE (1)..(9): IAPP 1-9
  PEPTIDE (10)..(13): KKK-epsilon K as a spacer
  SITE (13)..(13): epsilon K
  PEPTIDE (14)..(32): MvF5 Th (UBITh1)
KCNTATCATKKKKISITEIKGVIVHRIETILF

SEQ ID NO: 114 - protein, 29 aa, Artificial Sequence
  Synthetic peptide
  PEPTIDE (1)..(9): IAPP 1-9
  SITE (10)..(10): epsilon K as a spacer
  PEPTIDE (11)..(29): MvF5 Th (UBITh1)
KCNTATCATKISITEIKGVIVHRIETILF

SEQ ID NO: 115 - protein, 36 aa, Artificial Sequence
  Synthetic peptide
  PEPTIDE (1)..(13): IAPP 1-13
  PEPTIDE (14)..(17): KKK-epsilon K as a spacer
  SITE (17)..(17): epsilon K
  PEPTIDE (18)..(36): MvF5 Th (UBITh1)
KCNTATCATQRLAKKKKISITEIKGVIVHRIETILF

SEQ ID NO: 116 - protein, 39 aa, Artificial Sequence
  Synthetic peptide
  PEPTIDE (1)..(16): IAPP 1-16
  PEPTIDE (17)..(20): KKK-epsilon K as a spacer
  SITE (20)..(20): epsilon K
  PEPTIDE (21)..(39): MvF5 Th (UBITh1)
KCNTATCATQRLANFLKKKKISITEIKGVIVHRIETILF

SEQ ID NO: 117 - protein, 29 aa, Artificial Sequence
  Synthetic peptide
  PEPTIDE (1)..(6): IAPP 2-7
  PEPTIDE (7)..(10): KKK-epsilon K as a spacer
  SITE (10)..(10): epsilon K
  PEPTIDE (11)..(29): MvF5 Th (UBITh1)
CNTATCKKKKISITEIKGVIVHRIETILF

SEQ ID NO: 118 - protein, 26 aa, Artificial Sequence
  Synthetic peptide
  PEPTIDE (1)..(6): IAPP 2-7
  SITE (7)..(7): epsilon K as a spacer
  PEPTIDE (8)..(26): MvF5 Th (UBITh1)
CNTATCKISITEIKGVIVHRIETILF

SEQ ID NO: 119 - protein, 35 aa, Artificial Sequence
  Synthetic peptide
  PEPTIDE (1)..(12): IAPP 2-13
  PEPTIDE (13)..(16): KKK-epsilon K as a spacer
  SITE (16)..(16): epsilon K
  PEPTIDE (17)..(35): MvF5 Th (UBITh1)
CNTATCATQRLAKKKKISITEIKGVIVHRIETILF

SEQ ID NO: 120 - protein, 37 aa, Artificial Sequence
  Synthetic peptide
  PEPTIDE (1)..(14): IAPP 2-15
  PEPTIDE (15)..(18): KKK-epsilon K as a spacer
  SITE (18)..(18): epsilon K
  PEPTIDE (19)..(37): MvF5 Th (UBITh1)
CNTATCATQRLANFKKKKISITEIKGVIVHRIETILF

SEQ ID NO: 121 - protein, 34 aa, Artificial Sequence
  Synthetic peptide
  PEPTIDE (1)..(14): IAPP 2-15
  SITE (15)..(15): epsilon K as a spacer
  PEPTIDE (16)..(34): MvF5 Th (UBITh1)
CNTATCATQRLANFKISITEIKGVIVHRIETILF

SEQ ID NO: 122 - protein, 33 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(33): IAPP 11-20
ISITEIKGVIVHRIETILFKKKKRLANFLVHSS

SEQ ID NO: 123 - protein, 33 aa, Artificial Sequence
  Synthetic peptide
  PEPTIDE (1)..(10): IAPP 11-20
  PEPTIDE (11)..(14): KKK-epsilon K as a spacer
  SITE (14)..(14): epsilon K
  PEPTIDE (15)..(33): MvF5 Th (UBITh1)
RLANFLVHSSKKKKISITEIKGVIVHRIETILF

SEQ ID NO: 124 - protein, 33 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(33): IAPP 20-29
ISITEIKGVIVHRIETILFKKKKSNNFGAILSS

SEQ ID NO: 125 - protein, 33 aa, Artificial Sequence
  Synthetic peptide
  PEPTIDE (1)..(10): IAPP 20-29
  PEPTIDE (11)..(14): KKK-epsilon K as a spacer
  SITE (14)..(14): epsilon K
  PEPTIDE (15)..(33): MvF5 Th (UBITh1)
SNNFGAILSSKKKKISITEIKGVIVHRIETILF

SEQ ID NO: 126 - protein, 44 aa, Artificial Sequence
  Synthetic peptide
  PEPTIDE (1)..(21): IAPP 10-30
  PEPTIDE (22)..(25): KKK-epsilon K as a spacer
  SITE (25)..(25): epsilon K
  PEPTIDE (26)..(44): MvF5 Th (UBITh1)
QRLANFLVHSSNNFGAILSSTKKKKISITEIKGVIVHRIETILF

SEQ ID NO: 127 - protein, 44 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(44): IAPP 10-30
ISITEIKGVIVHRIETILFKKKKQRLANFLVHSSNNFGAILSST

SEQ ID NO: 128 - protein, 50 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(50): IAPP 11-37
ISITEIKGVIVHRIETILFKKKKRLANFLVHSSNNFGAILSSTNVGSNTY

SEQ ID NO: 129 - protein, 46 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(46): IAPP 15-37
ISITEIKGVIVHRIETILFKKKKFLVHSSNNFGAILSSTNVGSNTY

SEQ ID NO: 130 - protein, 36 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(36): IAPP 18-30
ISITEIKGVIVHRIETILFKKKKHSSNNFGAILSST

SEQ ID NO: 131 - protein, 39 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(39): IAPP 18-33
ISITEIKGVIVHRIETILFKKKKHSSNNFGAILSSTNVG

SEQ ID NO: 132 - protein, 41 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(41): IAPP 18-35
ISITEIKGVIVHRIETILFKKKKHSSNNFGAILSSTNVGSN

SEQ ID NO: 133 - protein, 43 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): IAPP 18-37
ISITEIKGVIVHRIETILFKKKKHSSNNFGAILSSTNVGSNTY

SEQ ID NO: 134 - protein, 41 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(41): IAPP 20-37
ISITEIKGVIVHRIETILFKKKKSNNFGAILSSTNVGSNTY

SEQ ID NO: 135 - protein, 38 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(38): IAPP 23-37
ISITEIKGVIVHRIETILFKKKKFGAILSSTNVGSNTY

SEQ ID NO: 136 - protein, 36 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(36): IAPP 25-37
ISITEIKGVIVHRIETILFKKKKAILSSTNVGSNTY

SEQ ID NO: 137 - protein, 31 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(31): IAPP 30-37
ISITEIKGVIVHRIETILFKKKKTNVGSNTY

SEQ ID NO: 138 - protein, 28 aa, Artificial Sequence
  Synthetic peptide
  PEPTIDE (1)..(19): MvF5 Th (UBITh1)
  SITE (20)..(20): epsilon-K as a spacer
  PEPTIDE (21)..(28): IAPP 30-37
ISITEIKGVIVHRIETILFKTNVGSNTY

SEQ ID NO: 139 - protein, 43 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(43): Guinea Pig IAPP 18-37
ISITEIKGVIVHRIETILFKKKKRSSHNLGAALLPTDVGSNTY

SEQ ID NO: 140 - protein, 42 aa, Artificial Sequence
  Synthetic peptide
  PEPTIDE (1)..(18): HBsAg3 Th (UBITh2)
  SITE (19)..(19): epsilon-K
  PEPTIDE (19)..(22): epsilon-K-KKK spacer
  PEPTIDE (23)..(42): IAPP 18-37
KKKIITITRIITIITTIDKKKKHSSNNFGAILSSTNVGSNTY

SEQ ID NO: 141 - protein, 43 aa, Artificial Sequence
  Synthetic peptide
  PEPTIDE (1)..(19): MvF4 Th  (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon-K-KKK spacer
  PEPTIDE (24)..(43): IAPP 18-37
ISIXEIXXVIVXXIETILFKKKKHSSNNFGAILSSTNVGSNTY

SEQ ID NO: 142 - protein, 41 aa, Artificial Sequence
  Synthetic peptide
  PEPTIDE (1)..(17): Clostridium tetani1 Th
  PEPTIDE (18)..(21): epsilon-K-KKK spacer
  SITE (18)..(18): epsilon-K
  PEPTIDE (22)..(41): IAPP 18-37
KKQYIKANSKFIGITELKKKKHSSNNFGAILSSTNVGSNTY

SEQ ID NO: 143 - protein, 39 aa, Artificial Sequence
  Synthetic peptide
  PEPTIDE (1)..(15): MvF1 Th
  PEPTIDE (16)..(19): epsilon-K-KKK spacer
  SITE (16)..(16): epsilon-K
  PEPTIDE (20)..(39): IAPP 18-37
LSEIKGVIVHRLEGVKKKKHSSNNFGAILSSTNVGSNTY

SEQ ID NO: 144 - protein, 48 aa, Artificial Sequence
  Synthetic peptide
  PEPTIDE (1)..(24): Bordetella pertussis Th
  PEPTIDE (25)..(28): epsilon-K-KKK spacer
  SITE (25)..(25): epsilon-K
  PEPTIDE (29)..(48): IAPP 18-37
GAYARCPNGTRALTVAELRGNAELKKKKHSSNNFGAILSSTNVGSNTY

SEQ ID NO: 145 - protein, 41 aa, Artificial Sequence
  Synthetic peptide
  PEPTIDE (1)..(17): Clostridium tetani2 Th
  PEPTIDE (18)..(21): epsilon-K-KKK spacer
  SITE (18)..(18): epsilon-K
  PEPTIDE (22)..(41): IAPP 18-37
WVRDIIDDFTNESSQKTKKKKHSSNNFGAILSSTNVGSNTY

SEQ ID NO: 146 - protein, 47 aa, Artificial Sequence
  Synthetic peptide
  PEPTIDE (1)..(23): Diphtheria Th
  PEPTIDE (24)..(27): epsilon-K-KKK spacer
  SITE (24)..(24): epsilon-K
  PEPTIDE (28)..(47): IAPP 18-37
DSETADNLEKTVAALSILPGHGCKKKKHSSNNFGAILSSTNVGSNTY

SEQ ID NO: 147 - protein, 45 aa, Artificial Sequence
  Synthetic peptide
  PEPTIDE (1)..(21): Plasmodium falciparum Th
  PEPTIDE (22)..(25): epsilon-K-KKK spacer
  SITE (22)..(22): epsilon-K
  PEPTIDE (26)..(45): IAPP 18-37
DHEKKHAKMEKASSVFNVVNSKKKKHSSNNFGAILSSTNVGSNTY

SEQ ID NO: 148 - protein, 41 aa, Artificial Sequence
  Synthetic peptide
  PEPTIDE (1)..(17): Schistosoma mansoni Th
  PEPTIDE (18)..(21): epsilon-K-KKK spacer
  SITE (18)..(18): epsilon-K
  PEPTIDE (22)..(41): IAPP 18-37
KWFKTNAPNGVDEKHRHKKKKHSSNNFGAILSSTNVGSNTY

SEQ ID NO: 149 - protein, 49 aa, Artificial Sequence
  Synthetic peptide
  PEPTIDE (1)..(25): Cholera Toxin Th
  PEPTIDE (26)..(29): epsilon-K-KKK spacer
  SITE (26)..(26): epsilon-K
  PEPTIDE (30)..(49): IAPP 18-37
ALNIWDRFDVFCTLGATTGYLKGNSKKKKHSSNNFGAILSSTNVGSNTY

SEQ ID NO: 150 - protein, 39 aa, Artificial Sequence
  Synthetic peptide
  PEPTIDE (1)..(15): MvF2 Th
  PEPTIDE (16)..(19): epsilon-K-KKK spacer
  SITE (16)..(16): epsilon-K
  PEPTIDE (20)..(39): IAPP 18-37
ISEIKGVIVHKIEGIKKKKHSSNNFGAILSSTNVGSNTY

SEQ ID NO: 151 - protein, 46 aa, Artificial Sequence
  Synthetic peptide
  PEPTIDE (1)..(22): KKKMvF3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
  SITE (23)..(23): epsilon K as a spacer
  PEPTIDE (23)..(26): epsilon-K-KKK spacer
  PEPTIDE (27)..(46): IAPP 18-37
KKKISIXEIXXVIVXXIEXILFKKKKHSSNNFGAILSSTNVGSNTY

SEQ ID NO: 152 - protein, 42 aa, Artificial Sequence
  Synthetic peptide
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
  SITE (19)..(19): epsilon K as a spacer
  PEPTIDE (19)..(22): epsilon-K-KKK spacer
  PEPTIDE (23)..(42): IAPP 18-37
XXXXXXXTXXXTXPXSXXKKKKHSSNNFGAILSSTNVGSNTY

SEQ ID NO: 153 - protein, 42 aa, Artificial Sequence
  Synthetic peptide
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
  SITE (19)..(19): epsilon K as a spacer
  PEPTIDE (19)..(22): epsilon-K-KKK spacer
  PEPTIDE (23)..(42): IAPP 18-37
KKKXXXXTRIXTIXXXXDKKKKHSSNNFGAILSSTNVGSNTY

SEQ ID NO: 154 - protein, 35 aa, Artificial Sequence
  Synthetic peptide
  PEPTIDE (1)..(11): Influenza MP1_1 Th
  PEPTIDE (12)..(15): epsilon-K-KKK spacer
  SITE (12)..(12): epsilon-K
  PEPTIDE (16)..(35): IAPP 18-37
FVFTLTVPSERKKKKHSSNNFGAILSSTNVGSNTY

SEQ ID NO: 155 - protein, 39 aa, Artificial Sequence
  Synthetic peptide
  PEPTIDE (1)..(15): Influenza MP1_2 Th
  PEPTIDE (16)..(19): epsilon-K-KKK spacer
  SITE (16)..(16): epsilon-K
  PEPTIDE (20)..(39): IAPP 18-37
SGPLKAEIAQRLEDVKKKKHSSNNFGAILSSTNVGSNTY

SEQ ID NO: 156 - protein, 33 aa, Artificial Sequence
  Synthetic peptide
  PEPTIDE (1)..(9): Influenza NSP1 Th
  PEPTIDE (10)..(13): epsilon-K-KKK spacer
  SITE (10)..(10): epsilon-K
  PEPTIDE (14)..(33): IAPP 18-37
DRLRRDQKSKKKKHSSNNFGAILSSTNVGSNTY

SEQ ID NO: 157 - protein, 43 aa, Artificial Sequence
  Synthetic peptide
  PEPTIDE (1)..(19): EBV BHRF1 Th
  PEPTIDE (20)..(23): epsilon-K-KKK spacer
  SITE (20)..(20): epsilon-K
  PEPTIDE (24)..(43): IAPP 18-37
AGLTLSLLVICSYLFISRGKKKKHSSNNFGAILSSTNVGSNTY

SEQ ID NO: 158 - protein, 39 aa, Artificial Sequence
  Synthetic peptide
  PEPTIDE (1)..(15): Clostridium tetani TT1 Th
  PEPTIDE (16)..(19): epsilon-K-KKK spacer
  SITE (16)..(16): epsilon-K
  PEPTIDE (20)..(39): IAPP 18-37
QYIKANSKFIGITELKKKKHSSNNFGAILSSTNVGSNTY

SEQ ID NO: 159 - protein, 44 aa, Artificial Sequence
  Synthetic peptide
  PEPTIDE (1)..(20): EBV EBNA-1 Th
  PEPTIDE (21)..(24): epsilon-K-KKK spacer
  SITE (21)..(21): epsilon-K
  PEPTIDE (25)..(44): IAPP 18-37
PGPLRESIVCYFMVFLQTHIKKKKHSSNNFGAILSSTNVGSNTY

SEQ ID NO: 160 - protein, 45 aa, Artificial Sequence
  Synthetic peptide
  PEPTIDE (1)..(21): Clostridium tetani TT2 Th
  PEPTIDE (22)..(25): epsilon-K-KKK spacer
  SITE (22)..(22): epsilon-K
  PEPTIDE (26)..(45): IAPP 18-37
FNNFTVSFWLRVPKVSASHLEKKKKHSSNNFGAILSSTNVGSNTY

SEQ ID NO: 161 - protein, 40 aa, Artificial Sequence
  Synthetic peptide
  PEPTIDE (1)..(16): Clostridium tetani TT3 Th
  PEPTIDE (17)..(20): epsilon-K-KKK spacer
  SITE (17)..(17): epsilon-K
  PEPTIDE (21)..(40): IAPP 18-37
KFIIKRYTPNNEIDSFKKKKHSSNNFGAILSSTNVGSNTY

SEQ ID NO: 162 - protein, 40 aa, Artificial Sequence
  Synthetic peptide
  PEPTIDE (1)..(16): Clostridium tetani TT4 Th
  PEPTIDE (17)..(20): epsilon-K-KKK spacer
  SITE (17)..(17): epsilon-K
  PEPTIDE (21)..(40): IAPP 18-37
VSIDKFRIFCKALNPKKKKKHSSNNFGAILSSTNVGSNTY

SEQ ID NO: 163 - protein, 42 aa, Artificial Sequence
  Synthetic peptide
  PEPTIDE (1)..(18): EBV CP Th
  PEPTIDE (19)..(22): epsilon-K-KKK spacer
  SITE (19)..(19): epsilon-K
  PEPTIDE (23)..(42): IAPP 18-37
VPGLYSPCRAFFNKEELLKKKKHSSNNFGAILSSTNVGSNTY

SEQ ID NO: 164 - protein, 38 aa, Artificial Sequence
  Synthetic peptide
  PEPTIDE (1)..(14): HCMV IE1 Th
  PEPTIDE (15)..(18): epsilon-K-KKK spacer
  SITE (15)..(15): epsilon-K
  PEPTIDE (19)..(38): IAPP 18-37
DKREMWMACIKELHKKKKHSSNNFGAILSSTNVGSNTY

SEQ ID NO: 165 - protein, 39 aa, Artificial Sequence
  Synthetic peptide
  PEPTIDE (1)..(15): EBV GP340 Th
  PEPTIDE (16)..(19): epsilon-K-KKK spacer
  SITE (16)..(16): epsilon-K
  PEPTIDE (20)..(39): IAPP 18-37
TGHGARTSTEPTTDYKKKKHSSNNFGAILSSTNVGSNTY

SEQ ID NO: 166 - protein, 37 aa, Artificial Sequence
  Synthetic peptide
  PEPTIDE (1)..(13): EBV BPLF1 Th
  PEPTIDE (14)..(17): epsilon-K-KKK spacer
  SITE (14)..(14): epsilon-K
  PEPTIDE (18)..(37): IAPP 18-37
KELKRQYEKKLRQKKKKHSSNNFGAILSSTNVGSNTY

SEQ ID NO: 167 - protein, 35 aa, Artificial Sequence
  Synthetic peptide
  PEPTIDE (1)..(11): EBV EBNA-2 Th
  PEPTIDE (12)..(15): epsilon-K-KKK spacer
  SITE (12)..(12): epsilon-K
  PEPTIDE (16)..(35): IAPP 18-37
TVFYNIPPMPLKKKKHSSNNFGAILSSTNVGSNTY

SEQ ID NO: 168 - protein, 37 aa, Homo sapiens
  PEPTIDE (1)..(37): Human ?-CGRP 1-37
ACDTATCVTHRLAGLLSRSGGVVKNNFVPTNVGSKAF

SEQ ID NO: 169 - protein, 42 aa, Homo sapiens [sequence omitted]

SEQ ID NO: 170 - protein, 51 aa, Homo sapiens
  PEPTIDE (1)..(51): IAPP -7 to 44
SHQVEKRKCNTATCATQRLANFLVHSSNNFGAILSSTNVGSNTYGKRNAVE

SEQ ID NO: 171 - protein, 16 aa, Measles virus
  PEPTIDE (1)..(16): MvF Th
DLSDLKGLLLHKLDGL

SEQ ID NO: 172 - protein, 16 aa, Measles virus
  PEPTIDE (1)..(16): MvF Th
EISDEIRLIIIKRIEI

SEQ ID NO: 173 - protein, 16 aa, Measles virus
  PEPTIDE (1)..(16): MvF Th
DVSDVKGVVVHKVDGV

SEQ ID NO: 174 - protein, 16 aa, Measles virus
  PEPTIDE (1)..(16): MvF Th
DFSDFKGFFFHKFDGF

SEQ ID NO: 175 - protein, 16 aa, Measles virus
  PEPTIDE (1)..(16): MvF Th
  SITE (1)..(1): D or E
  SITE (2)..(2): L or I or V or F
  SITE (4)..(4): D or E
  SITE (5)..(5): L or I or V or F
  SITE (6)..(6): K or R
  SITE (8)..(8): L or I or V or F
  SITE (9)..(9): L or I or V or F
  SITE (10)..(10): L or I or V or F
  SITE (12)..(12): K or R
  SITE (13)..(13): L or I or V or F
  SITE (14)..(14): D or E
  SITE (16)..(16): L or I or V or F
XXSXXXGXXXHXXXGX

SEQ ID NO: 176 - protein, 19 aa, Measles virus
  PEPTIDE (1)..(19): MvF3 Th
ISISEIKGVIVHKIEGILF

SEQ ID NO: 177 - protein, 19 aa, Measles virus
  PEPTIDE (1)..(19): MvF 3Th
ISITEIRTVIVTRIETILF

SEQ ID NO: 178 - protein, 19 aa, Measles virus
  PEPTIDE (1)..(19): MvF3 Th
  SITE (4)..(4): S or T
  SITE (7)..(7): K or R
  SITE (8)..(8): G or T
  SITE (12)..(12): H or T
  SITE (13)..(13): K or R
  SITE (16)..(16): G or T
ISIXEIXXVIVXXIEXILF

SEQ ID NO: 179 - protein, 22 aa, Measles virus
  PEPTIDE (1)..(22): KKKMvF5 Th (UBITh1a)
KKKISITEIKGVIVHRIETILF

SEQ ID NO: 180 - protein, 15 aa, Hepatitis B virus
  PEPTIDE (1)..(15): HBsAg4 Th (UBITh4)
FFLLTRILTIPQSLD

SEQ ID NO: 181 - protein, 18 aa, Hepatitis B virus
  PEPTIDE (1)..(18): KKK-HBsAg Th
KKKFFLLTRILTIPQSLD

SEQ ID NO: 182 - protein, 14 aa, Hepatitis B virus
  PEPTIDE (1)..(14): HBsAg Th
FFLLTRILTIPQSL

SEQ ID NO: 183 - DNA, 32 bp, Artificial Sequence
  CpG1 oligonucleotide ODN
tcgtcgttttgtcgttttgtcgttttgtcgtt

SEQ ID NO: 184 - DNA, 24 bp, Artificial Sequence
  CpG2 oligonucleotide ODN
  misc_feature (1)..(1): phosphorothioate group
tcgtcgttttgtcgttttgtcgtt

SEQ ID NO: 185 - DNA, 24 bp, Artificial Sequence
  CpG3 Oligonucleotide ODN
tcgtcgttttgtcgttttgtcgtt

SEQ ID NO: 186 - protein, 37 aa, Papio hamadryas
  PEPTIDE (1)..(37): IAPP Baboon
ICNTATCATQRLANFLVRSSNNFGTILSSTNVGSNTY

SEQ ID NO: 187 - protein, 37 aa, Ursus americanus
  PEPTIDE (1)..(37): IAPP Bear
KCNTATCATQRLANFLVRSGNNLGAILSPTNVGSNTY

SEQ ID NO: 188 - protein, 37 aa, Bos taurus
  PEPTIDE (1)..(37): IAPP Bovine
KCGTATCETQRLANFLAPSSNKLGAIFSPTKMGSNTY

SEQ ID NO: 189 - protein, 37 aa, Sus scrofa
  PEPTIDE (1)..(37): IAPP Porcine
KCNMATCATQHLANFLDRSRNNLGTIFSPTKVGSNTY

SEQ ID NO: 190 - protein, 37 aa, Canis lupus
  PEPTIDE (1)..(37): IAPP Dog
KCNTATCATQRLANFLVRTSNNLGAILSPTNVGSNTY

SEQ ID NO: 191 - protein, 37 aa, Mustela putorius
  PEPTIDE (1)..(37): IAPP Ferret
KCNTATCVTQRLANFLIHSSNNLGAILLPTDVGSNTY

SEQ ID NO: 192 - protein, 37 aa, Carassius auratus
  PEPTIDE (1)..(37): IAPP Goldfish
KCNTATCVTQRLADFLVRSSNTRGTVYAPTNVGANTY

(19) United States
(12) Patent Application Publication
(10) Pub. No.: US 2024/0400634 A1
(43) Pub. Date: Dec. 5, 2024

(54) PEPTIDE IMMUNOGENS TARGETING ISLET AMYLOID POLYPEPTIDE (IAPP) AND FORMULATIONS THEREOF FOR PREVENTION AND TREATMENT OF DISORDERS RELATED TO AGGREGATED IAPP

(71) Applicant: UNITED BIOMEDICAL, INC., Hauppauge, NY (US)

(72) Inventor: Chang Yi Wang, Cold Spring Harbor, NY (US)

(21) Appl. No.: 17/798,688

(22) PCT Filed: Feb. 11, 2021
(86) PCT No.: PCT/US2021/017530

§ 371 Date: Aug. 10, 2022

Related U.S. Application Data
(60) Provisional application No. 62/972,760, filed on Feb. 11, 2020.

(51) Int. Cl. C07K 14/575; A61K 39/00; C07K 16/26

(52) U.S. Cl. CPC C07K 14/575; A61K 39/0005; C07K 16/26; C07K 2317/33; C07K 2317/76

(57) ABSTRACT

The present disclosure is directed to peptide immunogen constructs targeting portions of Islet Amyloid Polypeptide (IAPP), compositions containing the constructs, antibodies elicited by the constructs, and methods for making and using the constructs and compositions thereof. The disclosed peptide immunogen constructs have more than about 30 amino acids and contain (a) a B cell epitope having about more than about 6 contiguous amino acid residues from the IAPP aggregation prone region of the full-length IAPP protein; (b) a heterologous Th epitope; and (c) an optional heterologous spacer. The disclosed IAPP peptide immunogen constructs stimulate the generation of highly specific antibodies directed IAPP for the prevention and/or treatment of disorders associated with aggregated IAPP.

The present application is a U.S. National Phase Application filed under 35 U.S.C. § 371, based on PCT International Patent Application No. PCT/US2021/017530, filed Feb. 11, 2021, that claims the benefit of U.S. Provisional Application Ser. No. 62/972,760, filed Feb. 11, 2020. The entire contents of these applications are incorporated herein by reference in their entireties.

### FIELD OF THE INVENTION

This disclosure relates to peptide immunogen constructs targeting Islet Amyloid Polypeptide (IAPP) and formulations thereof for prevention and/or treatment of disorders related to aggregated LAPP, including patients with diabetes mellitus type 1 (T1D) due to islet rejection following clinical pancreatic islet transplantation and those with diabetes mellitus type 2 (T2D).

### BACKGROUND OF THE INVENTION

Protein accumulation, modification, and aggregation are pathological aspects of numerous metabolic diseases including well known neurodegenerative diseases such as Huntington's, Alzheimer's (AD) and Parkinson's diseases (PD), and non-neuro metabolic diseases, such as diabetes mellitus type 1 (T1D), due to islet rejection following clinical pancreatic islet transplantation, and diabetes mellitus type 2 (T2D).

Islet amyloid polypeptide (IAPP or amylin) is a physiological peptide co-secreted with insulin by β-cells in the pancreas that forms fibrillar aggregates in pancreatic islets (also called islets of Langerhans) of T2D patients and has been suggested to play a role in the development of the disease. LAPP aggregates have also been found in pancreatic islets upon transplantation of isolated islets in patients with diabetes mellitus type 1 (T1D).

Pancreatic islets are composed of 65% to 80% β-cells, which produce and secrete insulin and LAPP that are essential for regulation of blood glucose levels and cell metabolism.

Human IAPP is a peptide hormone processed from a preprohormone, PreProIAPP (GenBank Accession No. AAA35983.1) (SEQ ID NO: 1), an 89 amino acid precursor produced in pancreatic β-cells, which is rapidly cleaved after translation into ProIAPP, a 67 amino acid peptide (SEQ ID NO: 2). ProIAPP undergoes additional proteolysis and post-translational modifications to generate IAPP (GenBank Accession No. 5MGQ_A) (SEQ ID NO: 3) (FIG. 1A). IAPP consists of 37 amino acids, with a disulfide bridge between cysteine residues 2 and 7 and an amidated C-terminus (FIG. 1B). IAPP has a highly conserved sequence across multiple organisms (FIG. 1C).

As described in Hayden, M. R., et al., 2001 and Hay, D. L., et al., 2015, human IAPP (hIAPP) expression is regulated together with insulin. Increased insulin production leads to increased hIAPP levels. hIAPP is released from pancreatic β-cells into the blood circulation and is involved in glycemic regulation through gastric emptying and satiety control, in synergy with insulin. Although hIAPP acts as a regulator of cell metabolism under physiological conditions, hIAPP can aggregate and form amyloid fibrils (IAPP amyloidosis) associated with β-cell failure, death, and reduced β-cell mass.

Several evidences point toward hIAPP amyloidosis as a major trigger for T2D pathogenesis, as described in the background section of U.S. Pat. No. 9,475,866 by Grimm:
a. Deposition of hIAPP fibrils is found in more than 90% of type-2 diabetes patients.
   b. hIAPP aggregation is toxic to β-cells and correlates with the reduction in insulin producing β-cells.
c. Transgenic mice models expressing hIAPP show pancreatic islet amyloid deposits and spontaneously develop T2D. They recapitulate the human disease with β-cell dysfunction, β-cell mass deficiency and β-cell loss, comparable to what was observed in the tissues from T2D patients, providing evidence for the contribution of human IAPP in the development of the disease.
d. Treatment interfering with hIAPP aggregation ameliorated the diabetic phenotype and increased animal life span.
e. hIAPP aggregation and amyloidosis is a prerequisite for toxicity to β-cells. The non-amyloidogenic rodent IAPP (rIAPP), which is unable to form fibrils as a result of six amino acid substitution, is nontoxic to β-cells.
f. In the development of the disease, pathological hIAPP aggregation found in human pancreatic islets may cause β-cell dysfunction and death associated with impairment of insulin secretion. Compensatory increase in β-cell mass along with insulin and amylin secretion to maintain normal blood glucose levels may favor the formation of toxic hIAPP oligomers and deposition of hIAPP fibrils.
g. While initial hIAPP oligomers are considered as the main cytotoxic species, the hIAPP fibril end product may also play a role in β-cell loss. hIAPP fibrils have also been observed in isolated pancreatic islets from donors and associated with β-cell loss following clinical pancreatic islets transplantation into individuals with type-1 diabetes.

The exact mechanism leading to hIAPP aggregation and amyloidosis in T2D is unknown.

Insulin resistance in T2D increases insulin secretion demand together with proIAPP cell content and hIAPP release leading to amyloidosis. Another proposed mechanism is the accumulation and aggregation of N-terminal unprocessed proIAPP caused by proteolysis failure in the setting of insulin resistance. ProIAPP is therefore also considered as an appropriate therapeutic target and a potential biomarker for diagnosis of patients prone to develop T2D. Previous studies by Brender, J. R., et al., 2008 have shown that the N-terminal region (aa1-19), rather than the amyloidogenic region (aa20-29), is primarily responsible for the interaction of the LAPP peptide with membranes. Low concentrations of the hIAPP₁₋₁₉ fragment induces membrane disruption to a near identical extent as the full-length peptide. Similar to the full-length peptide, hIAPP₁₋₁₉ exhibits a random coil conformation in solution and adopts an α-helical conformation upon binding to lipid membranes. However, unlike the full-length peptide, the hIAPP₁₋₁₉ fragment does not form amyloid fibers. Thus, membrane disruption can occur independently from amyloid formation in IAPP, and the sequences responsible for amyloid formation and membrane disruption are located in different regions of the peptide.

Sequence analysis utilizing a consensus aggregation propensity predictor verified that the aa8-17 and aa20-29 segments of IAPP present the highest predicted amyloidogenic potential (Louros, N. N., et al., 2017). Accordingly, mutations targeted to reduce the hydrophobic potential and amyloidogenic properties of the aa8-17 and aa20-29 segments were introduced. Significant alterations of the aa8-17 segment were incapable of inhibiting IAPP aggregation, supporting the hypothesis that this is not the only segment controlling IAPP aggregation. However, the hydrophobic nature of this segment most probably participates in the formation of the amyloid fibril core, since the presence of charged residues leads to 3-fold slower rate of IAPP fibrillation.

Diabetes mellitus is a group of metabolic diseases including T1D, T2D, and gestational diabetes. T2D, also named as adult-onset diabetes, obesity-related diabetes, and noninsulin-dependent diabetes mellitus (NIDDM), is the most common form of diabetes, accounting for about 90% of all cases. T2D is characterized by a decrease in the number of functional insulin-producing β-cells. Clinical features of T2D are high blood glucose levels and insulin resistance and/or deficiency. While the pathology progresses, it can lead to long-term complications such as cardiovascular disease, diabetic retinopathy leading to blindness, kidney failure, frequent infections, and amputations caused by poor circulation leading to a shorter life expectancy. The disease affects more than 300 million people worldwide resulting in more than a million deaths annually. Both genetic determinants and environmental factors lead to the development of the disease, with obesity, physical inactivity, and aging being the primary causes.

Current treatments for T2D include lifestyle management (diet and exercise) and pharmacological intervention such as metformin and insulin supply to decrease blood glucose levels by either stimulating the pancreas to release insulin or increasing insulin response. These treatments are based on improving symptoms, but lack durability.

New treatment strategies involving analogues of glucagon-peptide 1 (GLP-1) and inhibitors of GLP-1 inactivating enzyme dipeptidyl-peptidase 4 (DDP4) are based on the potent insulinotropic effect of GLP-1 and its effect to enhance β-cell proliferation. These treatment strategies cause an increase in insulin and IAPP release and have been shown to promote the development of islet amyloidosis in animal models. These new treatments could potentially aggravate islet amyloidosis and none of the available treatments have been shown to counteract the aggregation of hIAPP and the loss of pancreatic β-cells.

More recent strategies involve the development of anti-inflammatory drugs or antibodies targeting the IL-1 pathway due to the finding that hIAPP, especially the aggregated fibrils, would specifically induce the inflammasome-IL-1 system leading to activation of the innate immune system.

Prior studies on treatment therapies highlight the potential benefit associated with active or passive immunotherapeutic approach targeting hIAPP, in particular those that target aggregated hIAPP, including oligomers and fibrils, to reduce or inhibit undesired activation of the innate immune system for efficacious and safe therapies for the treatment of disorders associated with aggregated IAPP.

Passive immunization using antibodies that are optimized and affinity matured by the human immune system could provide a promising new therapeutic avenue with a high probability for excellent efficacy and safety. Although such monoclonal anti-IAPP antibodies may prove efficacious in immunotherapy of disorders related to aggregated IAPP, they are expensive and must be administered frequently to maintain sufficient suppression of oligomeric or aggregated IAPP levels in serum and body fluid to arrive at the clinical benefits derived therefrom.

In contrast, an active immunization strategy, through a vaccination approach, would provide a cost effective, site-directed immunotherapeutic treatment targeting oligomeric or aggregated IAPP that would be safe and well tolerated. Such an active immunization strategy remains an exciting new intervention and development for disorders related to aggregated IAPP.

Site-directed vaccine development has been plagued with a number of disadvantages and deficiencies associated with the conventional chemical conjugation method for hapten peptide/carrier protein immunogen preparation. In general, such methods of preparation involve complicated chemical coupling procedures, using costly pharmaceutical grade KLH or toxoid protein as the T helper cell carrier protein, where most of the antibodies elicited therefrom being directed against the carrier protein and not the targeted B cell epitope(s).

In view of the above mentioned limitations associated with the monoclonal antibody therapy and the conventional peptide/carrier protein vaccine preparations, there is clearly an unmet need to develop an efficacious immunotherapeutic composition that is capable of eliciting highly specific antibody responses against the specific site(s) on IAPP to (1) counteract the aggregation of hIAPP; (2) exhibit preferential binding to, and consequential removal of, the oligomeric or aggregated IAPPs; and (3) protect the pancreatic β-cells from toxic killing by the aggregated IAPP. Such immunotherapeutic peptide immunogen compositions and vaccine formulations thereof would allow easy patient administration and large-scale manufacturing to facilitate cost-effective global treatment of patients suffering from disorders related to aggregated IAPP, including patients with T1D and T2D.

Three review articles, and additional references cited therein, support statements made in the above background section are hereby incorporated by reference in their entireties. The first article (Wikipedia: Amylin) contains an updated review on IAPP; the second (Akter, R., et al., 2016) describes the structure, function, and pathophysiology of IAPP, and the third (U.S. Pat. No. 9,475,866 by Grimm) discusses the potential use of monoclonal antibodies for passive immunotherapy of IAPP related disorders.

### REFERENCES

1. AKTER, R., et al., “Islet Amyloid Polypeptide: Structure, Function, and Pathophysiology.” J. Diabetes Res., 2016:2798269, 18 pages (2016)
2. BOWER, R. L., et al., “Amylin structure-function relationships and receptor pharmacology: implications for amylin mimetic drug development.” British Journal of Pharmacology, 173(12):1883-1898 (2016)
3. BRENDER, J. R., et al., “Amyloid fiber formation and membrane disruption are separate processes localized in two distinct regions of IAPP, the type-2-diabetes-related peptide.” J. Am. Chem. Soc., 130(20):6424-9 (2008)
4. CAO, P., et al., “Aggregation of islet amyloid polypeptide: from physical chemistry to cell biology.” Curr. Opin. Struct. Biol. 23(1):82-89 (2013)
5. CHANG, J. C. C., et al., “Adjuvant activity of incomplete Freund's adjuvant.” Advanced Drug Delivery Reviews, 32(3):173-186 (1998)
6. FIELDS, G. B., et al., Chapter 3 in Synthetic Peptides: A User's Guide, ed. Grant, W.H. Freeman & Co., New York, NY, p.77 (1992)
7. GRIMM, J., et al., “Human islet amyloid polypeptide (hIAPP) specific antibodies and uses thereof.” U.S. Pat. No. 9,475,866 B2 (2016 Oct. 25)
8. HAY, D. L., et al., “Amylin: Pharmacology, Physiology, and Clinical Potential.” Pharmacol. Rev., 67(3):564-600 (2015)
9. HAYDEN, M. R., et al., “‘A’ is for Amylin and Amyloid in Type 2 Diabetes Mellitus.” JOP. J. Pancreas (Online), 2(4):124-139 (2001)
10. LOUROS, N. N., et al., “Tracking the amyloidogenic core of IAPP amyloid fibrils: Insights from micro-Raman spectroscopy.” Journal of Structural Biology. 199(2):140-152 (2017)
11. TRAGGIAI, E., et al., “An efficient method to make human monoclonal antibodies from memory B cells: potent neutralization of SARS coronavirus”, Nature Medicine, 10:871-875 (2004).
12. WIKIPEDIA, The Free Encyclopedia, “Amylin”, available at website: en.wikipedia.org/wiki/Amylin) (accessed Jan. 13, 2020)
13. WO 1990/014837, by VAN NEST, G., et al., “Adjuvant formulation comprising a submicron oil droplet emulsion.” (1990 Dec. 13)

### SUMMARY OF THE INVENTION

The present disclosure is directed to portions of the Islet Amyloid Polypeptide (IAPP) that can be used as B cell epitopes. The present disclosure is also directed to designer peptide immunogen constructs containing B cell epitopes from IAPP, compositions containing the peptide immunogen constructs, methods of making and using the peptide immunogen constructs, and antibodies produced by the peptide immunogen constructs.

One aspect of the present disclosure is directed to B cell epitopes from different portions of IAPP from various organisms. The disclosed B cell epitopes have between about 6 to about 28 amino acids derived from IAPP or proIAPP from human (SEQ ID NOs: 1 or 2) or from other organism (e.g., SEQ ID NOs: 3-7 and 186-192). In certain embodiments, the B cell epitope peptides have an amino acid sequence selected from SEQ ID NOs: 8-69, as shown in Table 1. The B cell epitope peptides can be derived from the N-terminal region responsible for interaction of IAPP peptide with the membrane (e.g., SEQ ID NOs: 8-14); the Central region of IAPP (e.g., SEQ ID NOs: 14-21); or the C-terminal region of IAPP (e.g., SEQ ID NOs: 22-26).

The disclosed B cell epitope peptides derived from IAPP or proIAPP can be linked through an optional heterologous spacer to a heterologous T helper cell (Th) epitope peptide to form a peptide immunogen construct. In certain embodiments, the heterologous spacer is any molecule or chemical structure capable of linking two amino acids and/or peptides together, which can include a chemical compound, a naturally occurring amino acid, a non-naturally occurring amino acid, or any combination thereof. The heterologous Th epitope can be any Th epitope that is capable of enhancing the immune response to the B cell epitope. In certain embodiments, the Th epitope is derived from pathogen proteins having the amino acid sequences of SEQ ID NOs: 73-112 and 171-182, as shown in Table 2.

The disclosed peptide immunogen constructs contain the IAPP B cell epitope peptide covalently linked, at either the N- or C-terminus through an optional heterologous spacer to the heterologous Th epitope. The disclosed peptide immunogen constructs, containing the B cell epitope and Th epitope, have 20 or more total amino acids. In certain embodiments, the peptide immunogen constructs have the amino acid sequences of SEQ ID NOs: 113-167, as shown in Table 3.

The disclosed IAPP peptide immunogen constructs, containing both designed B cell- and Th-epitope peptides, act together to stimulate the generation of highly specific antibodies directed against IAPP functional sites, including the N-terminal region responsible for interaction of IAPP peptide with the membrane and the aggregation prone regions, and are cross-reactive with the full-length oligomeric or aggregated IAPP sequences in human and other organisms (e.g., SEQ ID NOs: 3-7). Antibodies generated from the disclosed peptide immunogen constructs are capable of providing therapeutic immune responses to patients predisposed to, or suffering from, disorders related to IAPP aggregation.

Another aspect of the present disclosure is directed to peptide compositions, including pharmaceutical compositions, containing a IAPP peptide immunogen construct. The compositions can contain one or more IAPP peptide immunogen construct, pharmaceutically acceptable delivery carriers, adjuvants, and/or be formulated into a stabilized immunostimulatory complex using a CpG oligomer. In certain embodiments, a mixture of IAPP peptide immunogen constructs have heterologous Th epitopes derived from different pathogens that can be used to allow coverage of as broad a genetic background in patients leading to a higher percentage in responder rate upon immunization for the prevention and/or treatment of patients with IAPP mediated disorders, including T1D and T2D.

The present disclosure is also directed to antibodies against the disclosed IAPP peptide immunogen constructs. In particular, the IAPP peptide immunogen constructs of the present disclosure are able to stimulate the generation of highly specific functional antibodies that are cross-reactive with the full-length IAPP molecule. The disclosed antibodies bind with high specificity to IAPP without much, if any, directed to the heterologous Th epitopes employed for immunogenicity enhancement, which is in sharp contrast to antibodies produced using conventional KLH or toxoid proteins or other biological carriers used for such peptide immunogenicity enhancement. Thus, the disclosed IAPP peptide immunogen constructs are capable of breaking the immune tolerance against self-IAPP, with a high responder rate, compared to other peptide or protein immunogens. Based on their unique characteristics and properties, the disclosed antibodies elicited by the IAPP peptide immunogen constructs are capable of providing a prophylactic and immunotherapeutic approach to treating patients suffering from IAPP mediated disorders, including T1D and T2D.

In some embodiments, the disclosed antibodies are directed against the N-terminal region responsible for interaction of IAPP peptide with the membrane (e.g., SEQ ID NOs: 8-14); the Central region of IAPP (e.g., SEQ ID NOs: 14-21); or the C-terminal region of IAPP (e.g., SEQ ID NOs: 22-26). The highly specific antibodies elicited by the IAPP peptide immunogen constructs can (1) inhibit IAPP aggregation to oligomers or fibrils and (2) protect the cellular toxicity exerted by aggregated IAPP towards the β cells, thus leading to effective treatment of patients suffering from disorders related to IAPP aggregation, including T1D and T2D.

In a further aspect, the present invention provides human monoclonal antibodies against oligomeric or aggregated IAPP induced by patients receiving compositions containing IAPP peptide immunogen constructs of this disclosure. An efficient method to make human monoclonal antibodies from B cells isolated from the blood of a human patient is described by Traggiai, E., et al, 2004, which is incorporated by reference.

The present disclosure is also directed to methods of making and using the disclosed IAPP peptide immunogen constructs, compositions, and antibodies. The disclosed methods provide for the low cost manufacture and quality control of IAPP peptide immunogen constructs and compositions containing the constructs. The disclosed methods are also directed to preventing and/or treating subjects predisposed to, or suffering from IAPP mediated disorders, including T1D and T2D, using the disclosed IAPP peptide immunogen constructs and/or antibodies elicited from the IAPP peptide immunogen constructs. The disclosed methods also include dosing regimens, dosage forms, and routes for administering the IAPP peptide immunogen constructs to prevent and/or treat IAPP mediated disorders, including T1D and T2D.

### BRIEF DESCRIPTION OF THE DRAWINGS

FIGS. 1A-1C present the amino acid sequences of human PreProIAPP, human, ProIAPP, and IAPP from human and other organisms. FIGS. 1A presents the human sequence of IAPP (Amylin) (SEQ ID NO: 3) and its precursors PreProIAPP (SEQ ID NO: 1) and ProIAPP (SEQ ID NO: 2).

FIG. 1B depicts the general structure of IAPP. FIG. 1C presents a Clustal Omega (1.2.4) multiple sequence alignment of IAPP sequences from human, cat, macaque, rat/mouse, guinea pig, baboon, bear, bovine, porcine, dog, ferret, and goldfish. At the bottom of the alignment, the asterisk (*) indicates positions that have a single, fully-conserved residue; the colon (:) indicates conservation between residues having strongly similar properties; and the period (.) indicates conservation between residues having weakly similar properties.

FIG. 2 depicts the pathway from discovery to commercialization of high precision designer IAPP peptide immunogen constructs and formulations thereof for the treatment of disorders associated with aggregated IAPP.

FIG. 3 illustrates the design of IAPP peptide immunogen constructs (SEQ ID NOs: 113-138) used for testing of immunogenicity, in vitro functional properties, and in vivo efficacy in animals.

FIG. 4 depicts results from immunogenicity studies of IAPP peptide immunogen constructs (SEQ ID NOs: 113-138) with 9 weeks post initial immunization (WPI) immune sera from guinea pigs with IAPP B cell epitope peptides derived from the N-terminal, Central, and C-terminal regions of the IAPP molecule.

FIG. 5 depicts the IAPP monomer binding profiles by dot blot binding assay with guinea pig immune sera collected at 12 weeks post initial immunization (WPI) with corresponding IAPP peptide immunogen constructs.

FIG. 6 depicts the IAPP oligomer binding profiles by dot blot binding assay with guinea pig immune sera collected at 12 weeks post initial immunization (WPI) with corresponding IAPP peptide immunogen constructs.

FIG. 7 depicts the IAPP fibril binding profiles by dot blot binding assay with guinea pig immune sera collected at 12 weeks post initial immunization (WPI) with corresponding IAPP peptide immunogen constructs.

FIG. 8 depicts inhibition of IAPP aggregation by anti-IAPP antibodies from guinea pig immune sera collected at 12 weeks post initial immunization (WPI) with corresponding IAPP peptide immunogen constructs. Thioflavin T (ThT) fluorescence assay was performed to show inhibition of fibrillation from monomer to fibril, or from oligomer to fibril.

FIG. 9 depicts the relative cell viability percentage (top bar graph) and inhibition of cell toxicity percentage (bottom bar graph) towards RIN-m5Fs cells exhibited by 40 μM aggregated IAPP oligomers by anti-IAPP antibodies from guinea pig immune sera collected at 12 weeks post initial immunization (WPI) with corresponding IAPP peptide immunogen constructs. PBS was used as a control where no IAPP aggregates were added to the cell culture, thus giving 100% cell viability. A purified antibody preparation from pooled preimmune sera was used as a negative control, which gave maximal cell toxicity. The cytotoxicity inhibition percentage (%) was calculated for each antibody preparation, as shown in the table and the bottom bar graph.

FIG. 10 illustrates the experimental protocol for preventive efficacy evaluation of representative IAPP peptide immunogen constructs in hIAPP^{+/-} transgenic (Tg) mice with type II diabetes mellitus (T2D).

FIG. 11 illustrates the experimental protocol for therapeutic efficacy evaluation of representative IAPP peptide immunogen constructs hIAPP^{+/-} transgenic (Tg) mice with type II diabetes mellitus (T2D).

### DETAILED DESCRIPTION OF THE INVENTION

The present disclosure is directed to portions of the Islet Amyloid Polypeptide (IAPP) and proIAPP that can be used as B cell epitopes. The present disclosure is also directed to designer peptide immunogen constructs containing B cell epitopes from IAPP, compositions containing the peptide immunogen constructs, methods of making and using the peptide immunogen constructs, and antibodies produced by the peptide immunogen constructs.

One aspect of the present disclosure is directed to B cell epitopes from different portions of IAPP from various organisms. The disclosed B cell epitopes have between about 6 to about 28 amino acids derived from IAPP, preproIAPP, or proIAPP from human (SEQ ID NOs: 1-3) or from other organism (e.g., SEQ ID NOs: 4-7 and 186-192). In certain embodiments, the B cell epitope peptides have an amino acid sequence selected from SEQ ID NOs: 8-69, as shown in Table 1. The B cell epitope peptides can be derived from the N-terminal region responsible for interaction of IAPP peptide with the membrane (e.g., SEQ ID NOs: 8-14); the Central region of LAPP (e.g., SEQ ID NOs: 14-21); or the C-terminal region of IAPP (e.g., SEQ ID NOs: 22-26). In some embodiments, the B cell epitope peptides are derived from the IAPP aggregation prone regions spanning aa8-17 and aa20-29 (e.g., SEQ ID NOs: 8-10, 12-25, and 41-63).

The disclosed B cell epitope peptides derived from IAPP or proIAPP can be linked through an optional heterologous spacer to a heterologous T helper cell (Th) epitope peptide to form a peptide immunogen construct. In certain embodiments, the heterologous spacer is any molecule or chemical structure capable of linking two amino acids and/or peptides together, which can include a chemical compound, a naturally occurring amino acid, a non-naturally occurring amino acid, or any combination thereof. The heterologous Th epitope can be any Th epitope that is capable of enhancing the immune response to the B cell epitope. In certain embodiments, the Th epitope is derived from pathogen proteins having the amino acid sequences of SEQ ID NOs: 73-112 and 171-182, as shown in Table 2.

In certain embodiments, the heterologous Th epitopes employed to enhance the immunogenicity of the IAPP B cell epitope peptide are derived from natural pathogens EBV BPLF1 (SEQ ID NO: 111), EBV CP (SEQ ID NO: 108), Clostridium Tetani (SEQ ID NOs: 73, 76, 103, 105-107), Cholera Toxin (SEQ ID NO: 80), and Schistosoma mansoni (SEQ ID NO: 79), as well as those idealized artificial Th epitopes derived from Measles Virus Fusion protein (MVF 1 to 5) and Hepatitis B Surface Antigen (HBsAg 1 to 3) in the form of either single sequence or combinatorial sequences (e.g. SEQ ID NOs: 74, 81-98, and 171-182).

The disclosed peptide immunogen constructs contain the IAPP B cell epitope peptide covalently linked, at either the N- or C-terminus through an optional heterologous spacer to the heterologous Th epitope. The disclosed peptide immunogen constructs, containing the B cell epitope and Th epitope, have 20 or more total amino acids. In certain embodiments, the peptide immunogen constructs have the amino acid sequences of SEQ ID NOs: 113-167, as shown in Table 3.

The disclosed IAPP peptide immunogen constructs, containing both designed B cell- and Th-epitope peptides, act together to stimulate the generation of highly specific antibodies directed against IAPP sites that are prone to aggregation at the central to C-terminal regions of the IAPP molecule, offering therapeutic immune responses to patients predisposed to, or suffering from, disorders related to IAPP aggregation.

Another aspect of the present disclosure is directed to peptide compositions, including pharmaceutical compositions, containing a IAPP peptide immunogen construct. The compositions can contain one or more IAPP peptide immunogen construct, pharmaceutically acceptable delivery carriers, adjuvants, and/or be formulated into a stabilized immunostimulatory complex using a CpG oligomer. In certain embodiments, a mixture of IAPP peptide immunogen constructs have heterologous Th epitopes derived from different pathogens that can be used to allow coverage of as broad a genetic background in patients leading to a higher percentage in responder rate upon immunization for the prevention and/or treatment of disorders related to IAPP aggregation.

Synergistic enhancement in IAPP immunogen constructs can be observed in the peptide compositions of this disclosure. The antibody response derived from the administration of such compositions containing IAPP peptide immunogen constructs was mostly (>90%) focused on the desired cross-reactivity with the full-length oligomeric or aggregated IAPP against the IAPP site(s) prone to IAPP aggregation (SEQ ID NOs: 8-69) without much, if any, directed to the heterologous Th epitopes employed for immunogenicity enhancement. This is in sharp contrast to standard methods that use a conventional carrier protein, such as KLH, toxoid, or other biological carriers, for B cell epitope peptide immunogenicity enhancement.

The present disclosure is also directed to pharmaceutical compositions and formulations for the prevention and/or treatment of disorders related to IAPP aggregation. In some embodiments, pharmaceutical compositions comprising a stabilized immunostimulatory complex, which is formed by mixing a CpG oligomer with a peptide composition containing a mixture of IAPP peptide immunogen constructs through electrostatic association, to further enhance the IAPP peptide immunogenicity towards the desired cross-reactivity with the full-length oligomeric or aggregated IAPP (e.g., SEQ ID NOs: 3-7).

In other embodiments, pharmaceutical compositions comprising the disclosed IAPP peptide immunogen construct, or mixture of constructs, are formulated with pharmaceutically acceptable delivery vehicles or adjuvants, such as mineral salts, including Alum gel (ALHYDROGEL) or Aluminum phosphate (ADJU-PHOS) to form a suspension formulation, or with MONTANIDE™ ISA 51 or 720 as adjuvant to form water-in-oil emulsions, that can be used for the prevention and/or treatment of disorders related to IAPP aggregation.

The present disclosure is also directed to antibodies directed against the disclosed IAPP peptide immunogen constructs. In particular, the IAPP peptide immunogen constructs of the present disclosure are able to stimulate the generation of highly specific functional antibodies that are cross-reactive with the full-length oligomeric or aggregated IAPP molecule. The disclosed antibodies bind with high specificity to oligomeric or aggregated IAPP without much, if any, directed to the heterologous Th epitopes employed for immunogenicity enhancement, which is in sharp contrast to antibodies produced using conventional proteins or other biological carriers used for such peptide immunogenicity enhancement. Thus, the disclosed IAPP peptide immunogen constructs are capable of breaking the immune tolerance against self-IAPP, with a high responder rate, compared to other peptide or protein immunogens.

In some embodiments, the disclosed antibodies are directed against and specifically bind to the sites prone to IAPP aggregation on the central to C-terminal portions of the IAPP molecule (e.g., SEQ ID NOs: 14-25) when the peptide immunogen constructs are administered to a subject. The highly specific antibodies elicited by these IAPP peptide immunogen constructs can inhibit IAPP aggregation, leading to effective prevention and/or treatment of disorders related to IAPP aggregation. The highly specific antibodies elicited by the IAPP peptide immunogen constructs can (1) inhibit IAPP aggregation to oligomers or fibrils and (2) protect the cellular toxicity exerted by aggregated IAPP towards the 13 cells, thus leading to effective treatment of patients suffering from disorders related to IAPP aggregation.

Based on their unique characteristics and properties, the disclosed antibodies elicited by the IAPP peptide immunogen constructs are capable of providing a prophylactic immunotherapeutic approach to treating patients suffering from disorders related to IAPP aggregation.

In a further aspect, the present invention provides human monoclonal antibodies against oligomeric or aggregated IAPP induced by patients receiving compositions containing IAPP peptide immunogen constructs of this disclosure. An efficient method to make human monoclonal antibodies from B cells isolated from the blood of a human patient is described by Traggiai, E., et al., 2004, which is incorporated by reference.

The present disclosure is also directed to methods of making the disclosed IAPP peptide immunogen constructs, compositions, formulations and antibodies. The disclosed methods provide for the low-cost manufacture and quality control of IAPP peptide immunogen constructs and compositions and formulations containing the constructs, which can be used in methods for treating patients suffering from disorders related to IAPP aggregation.

The present disclosure also includes methods for preventing and/or treating subjects predisposed to, or suffering from, disorders related to IAPP aggregation using the disclosed IAPP peptide immunogen constructs and/or antibodies directed against the IAPP peptide immunogen constructs. The methods for preventing and/or treating disorders related to IAPP aggregation in a subject include administering to the subject a composition containing a disclosed IAPP peptide immunogen construct, or mixture of constructs. In certain embodiments, the compositions utilized in the methods contain a disclosed IAPP peptide immunogen construct in the form of a stable immunostimulatory complex with negatively charged oligonucleotides, such as CpG oligomers, through electrostatic association, which can be further supplemented with an adjuvant, for administration to patients suffering from disorders related to IAPP aggregation.

The disclosed methods also include dosing regimens, dosage forms, and routes for administering the IAPP peptide immunogen constructs and formulations thereof to prevent and/or treat disorders related to IAPP aggregation in a subject.

### General

The section headings used herein are for organizational purposes only and are not to be construed as limiting the subject matter described. All references or portions of references cited in this application are expressly incorporated by reference herein in their entirety for any purpose.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence, the phrase “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples disclosed herein are illustrative only and not intended to be limiting.

### IAPP Peptide Immunogen Construct

The present disclosure provides peptide immunogen constructs containing a B cell epitope peptide having about 6 to about 28 amino acids derived from the full-length IAPP sequence from human (SEQ ID NO: 3) or from other organisms (e.g., SEQ ID NOs: 4-7). In certain embodiments, the B cell epitope peptide has an amino acid sequence selected from SEQ ID NOs: 8-69, as shown in Table 1.

The B cell epitope can be covalently linked to a heterologous T helper cell (Th) epitope derived from a pathogen protein (e.g., SEQ ID NOs: 73-112 and 171-182, as shown in Table 2) directly or through an optional heterologous spacer. These constructs, containing both designed B cell- and Th-epitopes act together to stimulate the generation of highly specific antibodies that are cross-reactive with full-length oligomeric or aggregated LAPP (SEQ ID NO: 3-7) of various organisms.

The phrase “LAPP peptide immunogen construct” or “peptide immunogen construct”, as used herein, refers to a peptide with more than about 20 amino acids containing (a) a B cell epitope having more than about 6 contiguous amino acid residues from the full-length LAPP polypeptide (SEQ ID NOs: 3-7); (b) a heterologous Th epitope; and (c) an optional heterologous spacer.

In certain embodiments, the IAPP peptide immunogen construct can be represented by the formulae:
(Th)ₘ-(A)ₙ-(IAPP functional B cell epitope peptide)-X
   or
(IAPP functional B cell epitope peptide)-(A)ₙ-(Th)ₘ-X
   or
(Th)ₘ-(A)ₙ-(IAPP functional B cell epitope peptide)-(A)ₙ-(Th)ₘ-X
wherein
Th is a heterologous T helper epitope;
A is a heterologous spacer;
(IAPP functional B cell epitope peptide) is a B cell epitope peptide having from 6 to 28 amino acid residues from IAPP that is prone to causing aggregation of IAPP;
X is an α-COOH or α-CONH₂ of an amino acid;
m is from 1 to about 4; and
n is from 0 to about 10.

The IAPP peptide immunogen constructs of the present disclosure were designed and selected based on a number of rationales, including:
i. the IAPP B cell epitope peptide is non-immunogenic on its own to avoid autologous T cell activation;
ii. the IAPP B cell epitope peptide can be rendered immunogenic by using a protein carrier or a potent T helper epitope(s);
iii. when the IAPP B cell epitope peptide is rendered immunogenic and administered to a host, the peptide immunogen construct:
   a. elicits high titer antibodies preferentially directed against the IAPP B cell epitope(s) and not the protein carrier or T helper epitope(s);
   b. breaks immune tolerance in the immunized host and generates highly specific antibodies having cross-reactivity with the full-length oligomeric or aggregated IAPP (SEQ ID NOs: 3-7);
   c. generates highly specific antibodies capable of inhibiting aggregation of IAPP monomer or oligomer into IAPP fibril;
   d. generates highly specific antibodies capable of inhibiting the associated cytotoxicity exerted by aggregated IAPP to β cells; and
   e. generates highly specific antibodies capable of reducing in vivo aggregated IAPP and disorders caused by such aggregated IAPP.

The disclosed IAPP peptide immunogen constructs and formulations thereof can effectively function as a pharmaceutical composition or vaccine formulation to prevent and/or treat subjects predisposed to, or suffering from, disorders related to aggregated IAPP.

The various components of the disclosed IAPP peptide immunogen constructs are described in further detail below.

a. B Cell Epitope Peptide from IAPP

The present disclosure is directed to a novel peptide composition for the generation of high titer antibodies with specificity for the oligomeric or aggregated IAPP of multi-species (e.g., SEQ ID NOs: 3-7). The site-specificity of the peptide immunogen constructs minimizes the generation of antibodies that are directed to irrelevant sites on other regions of IAPP or irrelevant sites on carrier proteins, thus providing a high safety factor.

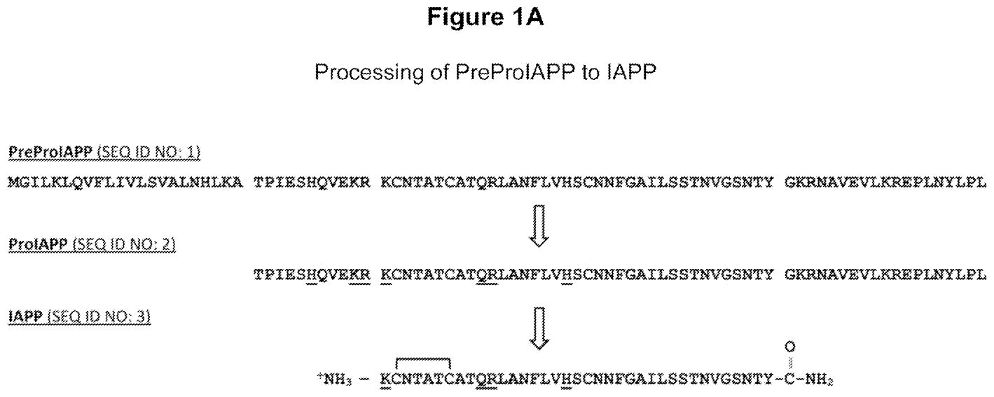

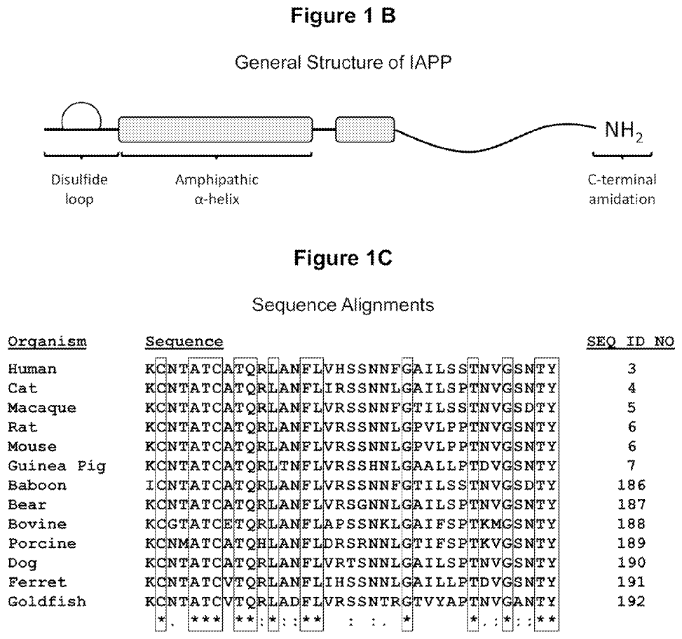

The term “IAPP”, as used herein, refers to Islet Amyloid Polypeptide, a peptide hormone that contains 37 amino acids, and has a disulfide bridge between cysteine residues 2 and 7, an amidated C-terminus, and has a highly conserved sequence across multiple species (FIGS. 1B and 1C). Human IAPP (hIAPP) is processed from a preprohormone, PreProIAPP (GenBank Accession No. AAA35983.1) (SEQ ID NO: 1), an 89 amino acid precursor produced in pancreatic β-cells, which is rapidly cleaved after translation into ProIAPP, a 67 amino acid peptide (SEQ ID NO: 2). ProIAPP undergoes additional proteolysis and post-translational modifications to generate IAPP (GenBank Accession No. 5MGQ_A) (SEQ ID NO: 3) (FIG. 1A). The amino acid sequences of IAPP for multiple species used in the present disclosure are shown in FIG. 1C.

One aspect of the present disclosure is to prevent and/or treat IAPP-disorders associated with aggregated IAPP with an active immunotherapy that preferentially targets oligomeric or aggregated IAPP to exert long-term clinical efficacy. Thus, the present disclosure is directed to peptide immunogen constructs targeting portions of the full-length IAPP polypeptide (SEQ ID NOs: 3-7) and formulations thereof for prevention and/or treatment of disorders associated with aggregated IAPP.

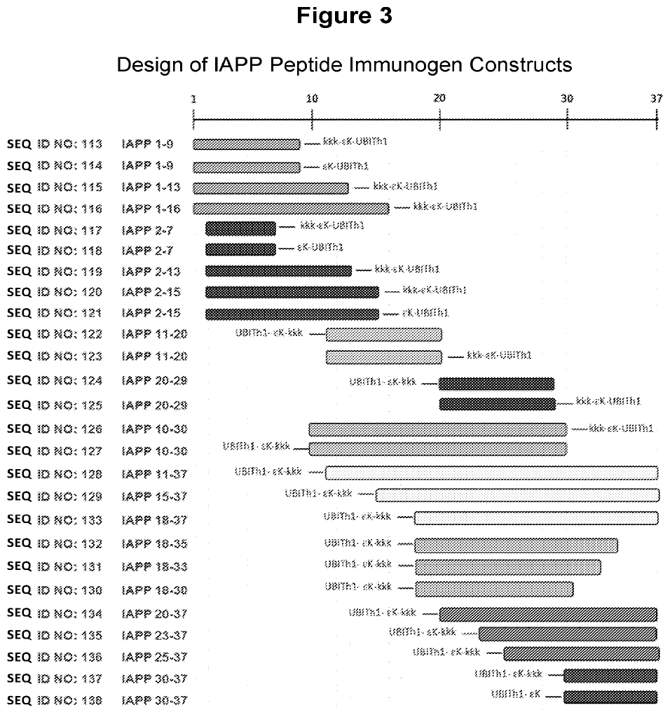

The B cell epitope portion of the IAPP peptide immunogen construct can contain between about 6 to about 28 amino acids. In some embodiments, the B cell epitope peptides have an amino acid sequence selected from SEQ ID NOs: 8-69, as shown in Table 1. In certain embodiments, the B cell epitope peptides can be derived from the N-terminal region responsible for interaction of IAPP peptide with the membrane (e.g., SEQ ID NOs: 8-14); the Central region of IAPP (e.g., SEQ ID NOs: 14-21); or the C-terminal region of IAPP (e.g., SEQ ID NOs: 22-26). In some embodiments, the B cell epitope peptides are derived from the IAPP aggregation prone regions spanning aa8-17 and aa20-29 (e.g., SEQ ID NOs: 8-10, 12-25, and 41-63). In other embodiments, the B cell epitope peptides are derived from the regions that exert cytotoxicity to 13 cells, which is located at the N-terminal, Central, to C-terminal regions of IAPP (e.g. SEQ ID NOs: 8-14, and 15-25, shown in Table 1 and FIG. 3).

The IAPP B cell epitope peptide of the present disclosure also includes immunologically functional analogues or homologues of IAPP, including IAPP sequences from different organisms (e.g., SEQ ID NOs: 3-7 and 186-192). Functional immunological analogues or homologues of IAPP B cell epitope peptides include variants that retain substantially the same immunogenicity as the original peptide. Immunologically functional analogues can have a conservative substitution in an amino acid position; a change in overall charge; a covalent attachment to another moiety; or amino acid additions, insertions, or deletions; and/or any combination thereof.

Antibodies generated from peptide immunogen constructs containing these B cell epitopes from IAPP are highly specific and cross-reactive with the full-length aggregated IAPP of various organisms (e.g., SEQ ID NOs: 3-7). Based on their unique characteristics and properties, the disclosed antibodies elicited by the IAPP peptide immunogen constructs are capable of providing a prophylactic immunotherapeutic approach to preventing and/or treating disorders related to aggregated IAPP.

b. Heterologous T Helper Cell Epitopes (Th Epitopes)

The present disclosure provides peptide immunogen constructs containing a B cell epitope from IAPP covalently linked to a heterologous T helper cell (Th) epitope directly or through an optional heterologous spacer.

The heterologous Th epitope in the peptide immunogen construct enhances the immunogenicity of the IAPP B cell epitope peptide, which facilitates the production of specific high titer antibodies directed against the optimized IAPP B cell epitope peptide screened and selected based on design rationales.

The term “heterologous”, as used herein, refers to an amino acid sequence that is derived from an amino acid sequence that is not part of, or homologous with, the wild-type sequence of IAPP. Thus, a heterologous Th epitope is a Th epitope derived from an amino acid sequence that is not naturally found in IAPP (i.e., the Th epitope is not autologous to IAPP). Since the Th epitope is heterologous to IAPP, the natural amino acid sequence of IAPP is not extended in either the N-terminal or C-terminal directions when the heterologous Th epitope is covalently linked to the IAPP B cell epitope peptide.

The heterologous Th epitope of the present disclosure can be any Th epitope that does not have an amino acid sequence naturally found in IAPP. The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e. little, if any, of the antibodies generated by the IAPP peptide immunogen constructs will be directed towards the Th epitope, thus allowing a very focused immune response directed to the targeted B cell epitope peptide of the IAPP molecule.

Th epitopes of the present disclosure include, but are not limited to, amino acid sequences derived from foreign pathogens, as exemplified in Table 2 (e.g., SEQ ID NOs: 73-112 and 171-182). In certain embodiments, the heterologous Th epitopes employed to enhance the immunogenicity of the IAPP B cell epitope peptide are derived from natural pathogens EBV BPLF1 (SEQ ID NO: 111), EBV CP (SEQ ID NO: 108), Clostridium Tetani (SEQ ID NOs: 73, 76, 103, 105-107), Cholera Toxin (SEQ ID NO: 80), and Schistosoma mansoni (SEQ ID NO: 79), as well as those idealized artificial Th epitopes derived from Measles Virus Fusion protein (MVF 1 to 5) and Hepatitis B Surface Antigen (HBsAg 1 to 3) in the form of either single sequence (e.g., SEQ ID NOs: 73-83, 85-89, 91-92, 94-95, 97-174, 176-177, 179-182) or combinatorial sequences (e.g., SEQ ID NOs: 84, 90, 93, 96, 175, and 178). The combinatorial idealized artificial Th epitopes contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 84, 90, 93, 96, 175, and 178, which are shown in Table 2. Th epitope peptides of the present invention provide broad reactivity and immunogenicity to animals and patients from genetically diverse populations.

c. Heterologous Spacer

The disclosed LAPP peptide immunogen constructs optionally contain a heterologous spacer that covalently links the IAPP B cell epitope peptide to the heterologous T helper cell (Th) epitope.

As discussed above, the term “heterologous”, refers to an amino acid sequence that is derived from an amino acid sequence that is not part of, or homologous with, the natural type sequence of IAPP. Thus, the natural amino acid sequence of IAPP is not extended in either the N-terminal or C-terminal directions when the heterologous spacer is covalently linked to the IAPP B cell epitope peptide because the spacer is heterologous to the IAPP sequence.

The spacer is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer can provide structural features to the IAPP peptide immunogen construct. Structurally, the spacer provides a physical separation of the Th epitope from the B cell epitope of the IAPP fragment. The physical separation by the spacer can disrupt any artificial secondary structures created by joining the Th epitope to the B cell epitope. Additionally, the physical separation of the epitopes by the spacer can eliminate interference between the Th cell and/or B cell responses. Furthermore, the spacer can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences encoding flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 70), where Xaa is any amino acid, and preferably aspartic acid.

The spacer can also provide functional features to the IAPP peptide immunogen construct. For example, the spacer can be designed to change the overall charge of the IAPP peptide immunogen construct, which can affect the solubility of the peptide immunogen construct. Additionally, changing the overall charge of the IAPP peptide immunogen construct can affect the ability of the peptide immunogen construct to associate with other compounds and reagents. As discussed in further detail below, the IAPP peptide immunogen construct can be formed into a stable immunostimulatory complex with a highly charged oligonucleotide, such as CpG oligomers, through electrostatic association. The overall charge of the IAPP peptide immunogen construct is important for the formation of these stable immunostimulatory complexes.

Chemical compounds that can be used as a spacer include, but are not limited to, (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

The spacer in the IAPP peptide immunogen construct can be covalently linked at either N- or C-terminal end of the Th epitope and the IAPP B cell epitope peptide. In some embodiments, the spacer is covalently linked to the C-terminal end of the Th epitope and to the N-terminal end of the IAPP B cell epitope peptide. In other embodiments, the spacer is covalently linked to the C-terminal end of the IAPP B cell epitope peptide and to the N-terminal end of the Th epitope. In certain embodiments, more than one spacer can be used, for example, when more than one Th epitope is present in the IAPP peptide immunogen construct. When more than one spacer is used, each spacer can be the same as each other or different. Additionally, when more than one Th epitope is present in the IAPP peptide immunogen construct, the Th epitopes can be separated with a spacer, which can be the same as, or different from, the spacer used to separate the Th epitope from the IAPP B cell epitope peptide. There is no limitation in the arrangement of the spacer in relation to the Th epitope or the IAPP B cell epitope peptide.

In certain embodiments, the heterologous spacer is a naturally occurring amino acid or a non-naturally occurring amino acid. In other embodiments, the spacer contains more than one naturally occurring or non-naturally occurring amino acid. In specific embodiments, the spacer is Lys-, Gly-, Lys-Lys-Lys-, (α, ε-N)Lys, ε-N-Lys-Lys-Lys-Lys (SEQ ID NO: 71), or Lys-Lys-Lys-ε-N-Lys (SEQ ID NO: 72).

d. Specific Embodiments of the IAPP Peptide Immunogen Constructs

In certain embodiments, the IAPP peptide immunogen constructs can be represented by the following formulae:
(Th)ₘ-(A)ₙ-(IAPP functional B cell epitope peptide)-X
   or
(IAPP functional B cell epitope peptide)-(A)ₙ-(Th)ₘ-X
   or
(Th)ₘ-(A)ₙ-(IAPP functional B cell epitope peptide)-(A)ₙ-(Th)ₘ-X
wherein
Th is a heterologous T helper epitope;
A is a heterologous spacer;
(IAPP functional B cell epitope peptide) is a B cell epitope peptide having from 6 to 28 amino acid residues from IAPP that is prone to causing aggregation of IAPP;
X is an α-COOH or α-CONH2 of an amino acid;
m is from 1 to about 4; and
n is from 0 to about 10.

The B cell epitope peptide can contain between about 6 to about 28 amino acids from portion of the full-length IAPP polypeptide represented by SEQ ID NOs: 3-7. In some embodiments, the B cell epitope has an amino acid sequence selected from any of SEQ ID NOs: 8-69, shown in Table 1. In certain embodiments, the B cell epitope peptide is from the N-terminal region responsible for interaction of IAPP peptide with the membrane (e.g., SEQ ID NOs: 8-14); the Central region of IAPP (e.g., SEQ ID NOs: 14-21); or the C-terminal region of IAPP (e.g., SEQ ID NOs: 22-26). In some embodiments, the B cell epitope peptides are derived from the IAPP aggregation prone regions spanning aa8-17 and aa20-29 (e.g., SEQ ID NOs: 8-10, 12-25, and 41-63). In other embodiments, the B cell epitope peptides are derived from the regions that exert cytotoxicity to β cells, which is located at the N-terminal, Central, to C-terminal regions of IAPP (e.g. SEQ ID NOs: 8-14, and 15-25, shown in Table 1 and FIG. 3).

The heterologous Th epitope in the IAPP peptide immunogen construct has an amino acid sequence selected from any of SEQ ID NOs: 73-112 and 171-182, and combinations thereof, shown in Table 2. In some embodiments, more than one Th epitope is present in the IAPP peptide immunogen construct.

The optional heterologous spacer is selected from any of Lys-, Gly-, Lys-Lys-Lys-, (α, ε-N)Lys, Pro-Pro-Xaa-Pro-Xaa-Pro (SEQ ID NO: 70), ε-N-Lys-Lys-Lys-Lys (SEQ ID NO: 71), Lys-Lys-Lys-ε-N-Lys (SEQ ID NO: 72), and any combination thereof, where Xaa is any amino acid, but preferably aspartic acid. In specific embodiments, the heterologous spacer is ε-N-Lys-Lys-Lys-Lys (SEQ ID NO: 71) or Lys-Lys-Lys-ε-N-Lys (SEQ ID NO: 72).

In certain embodiments, the IAPP peptide immunogen construct has an amino acid sequence selected from any of SEQ ID NOs: 113-167 as shown in Table 3.

The IAPP peptide immunogen constructs comprising Th epitopes are produced simultaneously in a single solid-phase peptide synthesis in tandem with the IAPP fragment. Th epitopes also include immunological analogues of Th epitopes. Immunological Th analogues include immune-enhancing analogues, cross-reactive analogues and segments of any of these Th epitopes that are sufficient to enhance or stimulate an immune response to the IAPP B cell epitope peptide.

The Th epitope in the IAPP peptide immunogen construct can be covalently linked at either N- or C-terminal end of the IAPP B cell epitope peptide. In some embodiments, the Th epitope is covalently linked to the N-terminal end of the IAPP B cell epitope peptide. In other embodiments, the Th epitope is covalently linked to the C-terminal end of the IAPP B cell epitope peptide. In certain embodiments, more than one Th epitope is covalently linked to the IAPP B cell epitope peptide. When more than one Th epitope is linked to the IAPP B cell epitope peptide, each Th epitope can have the same amino acid sequence or different amino acid sequences. In addition, when more than one Th epitope is linked to the IAPP B cell epitope peptide, the Th epitopes can be arranged in any order. For example, the Th epitopes can be consecutively linked to the N-terminal end of the IAPP B cell epitope peptide, or consecutively linked to the C-terminal end of the IAPP B cell epitope peptide, or a Th epitope can be covalently linked to the N-terminal end of the IAPP B cell epitope peptide while a separate Th epitope is covalently linked to the C-terminal end of the IAPPB cell epitope peptide. There is no limitation in the arrangement of the Th epitopes in relation to the IAPP B cell epitope peptide.

In some embodiments, the Th epitope is covalently linked to the IAPP B cell epitope peptide directly. In other embodiments, the Th epitope is covalently linked to the IAPP fragment through a heterologous spacer.

e. Variants, Homologues, and Functional Analogues

Variants and analogues of the above immunogenic peptide constructs that induce and/or cross-react with antibodies to the preferred IAPP B cell epitope peptides can also be used. Analogues, including allelic, species, and induced variants, typically differ from naturally occurring peptides at one, two, or a few positions, often by virtue of conservative substitutions. Analogues typically exhibit at least 75%, 80%, 85%, 90%, or 95% sequence identity with natural peptides. Some analogues also include unnatural amino acids or modifications of N- or C-terminal amino acids at one, two, or a few positions.

Variants that are functional analogues can have a conservative substitution in an amino acid position; a change in overall charge; a covalent attachment to another moiety; or amino acid additions, insertions, or deletions; and/or any combination thereof.

Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

In a particular embodiment, the functional analogue has at least 50% identity to the original amino acid sequence. In another embodiment, the functional analogue has at least 80% identity to the original amino acid sequence. In yet another embodiment, the functional analogue has at least 85% identity to the original amino acid sequence. In still another embodiment, the functional analogue has at least 90% identity to the original amino acid sequence.

Functional immunological analogues of the Th epitope peptides are also effective and included as part of the present invention. Functional immunological Th analogues can include conservative substitutions, additions, deletions and insertions of from one to about five amino acid residues in the Th epitope which do not essentially modify the Th-stimulating function of the Th epitope. The conservative substitutions, additions, and insertions can be accomplished with natural or non-natural amino acids, as described above for the LAPP B cell epitope peptide. Table 2 identifies another variation of a functional analogue for Th epitope peptide. In particular, SEQ ID NOs: 74 and 81 of MvF1 and MvF2 Th are functional analogues of SEQ ID NOs: 91-92 and 97 of MvF4 and MvF5, respectively, in that they differ in the amino acid frame by the deletion (SEQ ID NOs: 74 and 81) or the inclusion (SEQ ID NOs: 91-92 and 97) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-4 Ths (SEQ ID NOs: 74, 81, 82-84, 91-93, 97, and 171-179) and from Hepatitis Surface protein HBsAg 1-3 Ths (SEQ ID NOs: 85-90, 94-96, 98, and 180-182).

### Compositions

The present disclosure also provides compositions comprising the disclosed IAPP immunogen peptide constructs.

a. Peptide Compositions

Compositions containing the disclosed IAPP peptide immunogen constructs can be in liquid or solid/lyophilized form. Liquid compositions can include water, buffers, solvents, salts, and/or any other acceptable reagent that does not alter the structural or functional properties of the IAPP peptide immunogen constructs. Peptide compositions can contain one or more of the disclosed IAPP peptide immunogen constructs.

b. Pharmaceutical Compositions

The present disclosure is also directed to pharmaceutical compositions containing the disclosed IAPP peptide immunogen constructs.

Pharmaceutical compositions can contain carriers and/or other additives in a pharmaceutically acceptable delivery system. Accordingly, pharmaceutical compositions can contain a pharmaceutically effective amount of an IAPP peptide immunogen construct together with pharmaceutically-acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the IAPP peptide immunogen constructs without having any specific antigenic effect itself. Adjuvants used in the pharmaceutical composition can include oils, oil emulsions, aluminum salts, calcium salts, immune stimulating complexes, bacterial and viral derivatives, virosomes, carbohydrates, cytokines, polymeric microparticles. In certain embodiments, the adjuvant can be selected from alum (potassium aluminum phosphate), aluminum phosphate (e.g. ADJU-PHOS®), aluminum hydroxide (e.g. ALHYDROGEL®), calcium phosphate, incomplete Freund's adjuvant (IFA), Freund's complete adjuvant, MF59, adjuvant 65, Lipovant, ISCOM, liposyn, saponin, squalene, L121, EMULSIGEN®, EmulsIL-6n®, monophosphoryl lipid A (MPL), Quil A, QS21, MONTANIDE® ISA 35, ISA 50V, ISA 50V2, ISA 51, ISA 206, ISA 720, liposomes, phospholipids, peptidoglycan, lipopolysaccahrides (LPS), ASO1, ASO2, ASO3, ASO4, AF03, lipophilic phospholipid (lipid A), gamma inulin, algammulin, glucans, dextrans, glucomannans, galactomannans, levans, xylans, dimethyldioctadecylammonium bromide (DDA), as well as the other adjuvants and emulsifiers.

In some embodiments, the pharmaceutical composition contains MONTANIDE™ ISA 51 (an oil adjuvant composition comprised of vegetable oil and mannide oleate for production of water-in-oil emulsions), TWEEN® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e. w/o/w) emulsion with EMULSIGEN or EMULSIGEN D as the adjuvant.

Pharmaceutical compositions can also include pharmaceutically acceptable additives or excipients. For example, pharmaceutical compositions can contain antioxidants, binders, buffers, bulking agents, carriers, chelating agents, coloring agents, diluents, disintegrants, emulsifying agents, fillers, gelling agents, pH buffering agents, preservatives, solubilizing agents, stabilizers, and the like.

Pharmaceutical compositions can be formulated as immediate release or for sustained release formulations. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the IAPP peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for subcutaneous, intradermal, or intramuscular administration. Pharmaceutical compositions suitable for other modes of administration can also be prepared, including oral and intranasal applications.

Pharmaceutical compositions can also be formulated in a suitable dosage unit form. In some embodiments, the pharmaceutical composition contains from about 0.1 μg to about 1 mg of the IAPP peptide immunogen construct per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight and general health of the subject as is well known in the therapeutic arts.

In some embodiments, the pharmaceutical composition contains more than one IAPP peptide immunogen construct. A pharmaceutical composition containing a mixture of more than one IAPP peptide immunogen construct to allow for synergistic enhancement of the immunoefficacy of the constructs. Pharmaceutical compositions containing more than one IAPP peptide immunogen construct can be more effective in a larger genetic population due to a broad MHC class II coverage thus provide an improved immune response to the IAPP peptide immunogen constructs.

In some embodiments, the pharmaceutical composition contains an IAPP peptide immunogen construct selected from SEQ ID NOs: 113-167 (Table 3), as well as homologues, analogues and/or combinations thereof.

In certain embodiments, IAPP peptide immunogen constructs (SEQ ID NOs: 141, 151-153) with heterologous Th epitopes derived from MVF and HBsAg in a combinatorial form (SEQ ID NOs: 93, 84, 90, and 96) were mixed in an equimolar ratio for use in a formulation to allow for maximal coverage of a host population having a diverse genetic background.

Furthermore, the antibody response elicited by the IAPP peptide immunogen constructs (e.g. utilizing UBITh®1; SEQ ID NOs: 136 and 137) were mostly (>90%) focused on the desired cross-reactivity against the B cell epitope peptide of IAPP without much, if any, directed to the heterologous Th epitopes employed for immunogenicity enhancement (Tables 4 and 6). This is in sharp contrast to the conventional protein such as KLH or other biological protein carriers used for such IAPP peptide immunogenicity enhancement.

In other embodiments, pharmaceutical compositions comprising a peptide composition of, for example, a mixture of the IAPP peptide immunogen constructs in contact with mineral salts including Alum gel (ALHYDROGEL) or Aluminum phosphate (ADJUPHOS) as adjuvant to form a suspension formulation was used for administration to hosts.

Pharmaceutical compositions containing an IAPP peptide immunogen construct can be used to elicit an immune response and produce antibodies in a host upon administration.

c. Immunostimulatory Complexes

The present disclosure is also directed to pharmaceutical compositions containing an IAPP peptide immunogen construct in the form of an immunostimulatory complex with a CpG oligonucleotide. Such immunostimulatory complexes are specifically adapted to act as an adjuvant and/or as a peptide immunogen stabilizer. The immunostimulatory complexes are in the form of a particulate, which can efficiently present the IAPP peptide immunogen to the cells of the immune system to produce an immune response. The immunostimulatory complexes may be formulated as a suspension for parenteral administration. The immunostimulatory complexes may also be formulated in the form of water in oil (w/o) emulsions, as a suspension in combination with a mineral salt or with an in-situ gelling polymer for the efficient delivery of the IAPP peptide immunogen construct to the cells of the immune system of a host following parenteral administration.

The stabilized immunostimulatory complex can be formed by complexing an IAPP peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the IAPP peptide immunogen construct is designed to contain a cationic portion that is positively charged at a pH in the range of 5.0 to 8.0. The net charge on the cationic portion of the IAPP peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the IAPP peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the IAPP peptide immunogen construct is the heterologous spacer. In certain embodiments, the cationic portion of the IAPP peptide immunogen construct has a charge of +4 when the spacer sequence is (α, ε-N)Lys, (α,ε-N)-Lys-Lys-Lys-Lys (SEQ ID NO: 71), or Lys-Lys-Lys-ε-N-Lys (SEQ ID NO: 72).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. Preferably, the CpG immunostimulatory single-stranded DNA molecules contain 18-48 nucleotide bases, with the number of repeats of CpG motif in the range of 3 to 8.

More preferably the anionic oligonucleotide is represented by the formula: 5′ X¹CGX²3′ wherein C and G are unmethylated; and X¹ is selected from the group consisting of A (adenine), G (guanine) and T (thymine); and X² is C (cytosine) or T (thymine). Or, the anionic oligonucleotide is represented by the formula: 5′ (X³)₂CG(X⁴)₂3′ wherein C and G are unmethylated; and X³ is selected from the group consisting of A, T or G; and X⁴ is C or T. In specific embodiments, the CpG oligonucleotide has the sequence of CpG1: 5′TCg TCg TTT TgT CgT TTT gTC gTT TTg TCg TT 3′ (fully phosphorothioated) (SEQ ID NO: 183), CpG2: 5′ Phosphate TCg TCg TTT TgT CgT TTT gTC gTT 3′ (fully phosphorothioated) (SEQ ID NO: 184), or CpG3 5′ TCg TCg TTT TgT CgT TTT gTC gTT 3′ (fully phosphorothioated) (SEQ ID NO: 185).

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

The present disclosure is also directed to pharmaceutical compositions, including formulations, for the prevention and/or treatment of disorders associated with aggregated IAPP. In some embodiments, pharmaceutical compositions comprising a stabilized immunostimulatory complex, which is formed through mixing a CpG oligomer with a peptide composition containing a mixture of the IAPP peptide immunogen constructs (e.g., SEQ ID NOs: 113-167) through electrostatic association, to further enhance the immunogenicity of the IAPP peptide immunogen constructs and elicit antibodies that are cross-reactive with the IAPP proteins of SEQ ID NOs: 3-7 that are directed at the IAPP aggregation prone region.

In yet other embodiments, pharmaceutical compositions contain a mixture of the IAPP peptide immunogen constructs (e.g., any combination of SEQ ID NOs: 113-167) in the form of a stabilized immunostimulatory complex with CpG oligomers that are, optionally, mixed with mineral salts, including Alum gel (ALHYDROGEL) or Aluminum phosphate (ADJUPHOS) as an adjuvant with high safety factor, to form a suspension formulation for administration to hosts.

### Antibodies

The present disclosure also provides antibodies elicited by the IAPP peptide immunogen constructs.

The present disclosure provides IAPP peptide immunogen constructs and formulations thereof, cost effective in manufacturing, optimal in their design that are capable of eliciting high titer antibodies targeting the oligomeric or aggregated IAPP that is capable of breaking the immune tolerance against self-protein IAPP with a high responder rate in immunized hosts. The antibodies generated by the IAPP peptide immunogen constructs have high affinity towards the oligomeric or aggregated IAPP.

In some embodiments, IAPP peptide immunogen constructs for eliciting antibodies comprise a hybrid of a IAPP peptide targeting the IAPP site that is around (1) the IAPP aggregation prone regions spanning aa8-17 and aa20-29 (e.g., SEQ ID NOs: 8-10, 12-25, and 41-63); (2) the N-terminal region (aa1-19) responsible for interaction of the IAPP peptide with membrane (SEQ ID NOs: 8-14); or (3) the regions that exert cytotoxicity to β cells, which is located at the N-terminal, Central, to C-terminal regions of LAPP (e.g. SEQ ID NOs: 8-14, and 15-25, shown in Table 1 and FIG. 3), which is linked to a heterologous Th epitope derived from pathogenic proteins such as Measles Virus Fusion (MVF) protein and others (e.g., SEQ ID NOs: 73-112 and 171-182) through an optional heterologous spacer. The B cell epitope and Th epitope peptide of the LAPP peptide immunogen constructs act together to stimulate the generation of highly specific antibodies cross-reactive with the oligomeric or aggregated IAPP from human and other organisms (e.g., SEQ ID NO: 3-7).

Traditional methods for immunopotentiating a peptide, such as through chemical coupling to a carrier protein, for example, Keyhole Limpet Hemocyanin (KLH) or other carrier proteins such as Diphtheria toxoid (DT) and Tetanus Toxoid (TT) proteins, typically result in the generation of a large amount of antibodies directed against the carrier protein. Thus, a major deficiency of such peptide-carrier protein compositions is that most (>90%) of antibodies generated by the immunogen are the non-functional antibodies directed against the carrier protein KLH, DT or TT, which can lead to epitopic suppression.

Unlike the traditional method for immunopotentiating a peptide, the antibodies generated by the disclosed IAPP peptide immunogen constructs (e.g., SEQ ID NOs: 113-167) bind with highly specificity to the oligomeric or aggregated IAPP (e.g., SEQ ID NO: 3-7) with little, if any, antibodies directed against the heterologous Th epitope (e.g., SEQ ID NOs: 73-112 and 171-182) or the optional heterologous spacer. An example is shown in Table 4 and Table 6 for SEQ ID NOs: 136 and 137, which demonstrates that the antibodies generated from these peptide immunogen constructs are specifically directed against the B cell epitope and not the Th epitope or the CpG oligonucleotide.

### Methods

The present disclosure is also directed to methods for making and using the IAPP peptide immunogen constructs, compositions, and pharmaceutical compositions.

a. Methods for Manufacturing the IAPP Peptide Immunogen Construct

The IAPP peptide immunogen constructs of this disclosure can be made by chemical synthesis methods well known to the ordinarily skilled artisan (see, e.g., Fields, G. B., et al., 1992). The IAPP peptide immunogen constructs can be synthesized using the automated Merrifield techniques of solid phase synthesis with the α-NH2 protected by either t-Boc or F-moc chemistry using side chain protected amino acids on, for example, an Applied Biosystems Peptide Synthesizer Model 430A or 431. Preparation of IAPP peptide immunogen constructs comprising combinatorial library peptides for Th epitopes can be accomplished by providing a mixture of alternative amino acids for coupling at a given variable position.

After complete assembly of the desired LAPP peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to one of ordinary skill in the art.

The quality of peptides produced by this chemical process can be controlled and defined and, as a result, reproducibility of LAPP peptide immunogen constructs, immunogenicity, and yield can be assured. A detailed description of the manufacturing of the IAPP peptide immunogen construct through solid phase peptide synthesis is provided in Example 1.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically-derived drugs.

Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The IAPP peptide immunogen constructs can also be made using recombinant DNA technology including nucleic acid molecules, vectors, and/or host cells. As such, nucleic acid molecules encoding the IAPP peptide immunogen construct and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the IAPP peptide immunogen construct and immunologically functional analogues thereof. For example, methods can include a step of incubating a host cell containing an expression vector containing a nucleic acid molecule encoding an IAPP peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

b. Methods for the Manufacturing of Immunostimulatory Complexes

Various exemplary embodiments also encompass methods of producing the immunostimulatory complexes comprising LAPP peptide immunogen constructs and CpG oligodeoxynucleotide (ODN) molecule. Stabilized immunostimulatory complexes (ISC) are derived from a cationic portion of the LAPP peptide immunogen construct and a polyanionic CpG ODN molecule. The self-assembling system is driven by electrostatic neutralization of charge. Stoichiometry of the molar charge ratio of cationic portion of the IAPP peptide immunogen construct to anionic oligomer determines extent of association. The non-covalent electrostatic association of IAPP peptide immunogen construct and CpG ODN is a completely reproducible process. The peptide/CpG ODN immunostimulatory complex aggregates, which facilitate presentation to the “professional” antigen presenting cells (APC) of the immune system thus further enhancing the immunogenicity of the complexes. These complexes are easily characterized for quality control during manufacturing. The peptide/CpG ISC are well tolerated in vivo. This novel particulate system comprising CpG ODN and IAPP peptide immunogen constructs was designed to take advantage of the generalized B cell mitogenicity associated with CpG ODN use, yet promote balanced Th-1/Th-2 type responses.

The CpG ODN in the disclosed pharmaceutical compositions is 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen presenting cells (APC). Consequently, such formulations are novel conceptually and offer potential advantages by promoting the stimulation of immune responses by alternative mechanisms.

c. Methods for the Manufacturing of Pharmaceutical Compositions

Various exemplary embodiments also encompass pharmaceutical compositions containing IAPP peptide immunogen constructs. In certain embodiments, the pharmaceutical compositions employ water in oil emulsions and in suspension with mineral salts.

In order for a pharmaceutical composition to be used by a large population, safety becomes another important factor for consideration. Despite there has been use of water-in-oil emulsions in many clinical trials, Alum remains the major adjuvant for use in formulations due to its safety. Alum or its mineral salts Aluminum phosphate (ADJUPHOS) are, therefore, frequently used as adjuvants in preparation for clinical applications.

Other adjuvants and immunostimulating agents include 3 De-O-acylated monophosphoryl lipid A (MPL) or 3-DMP, polymeric or monomeric amino acids, such as polyglutamic acid or polylysine. Such adjuvants can be used with or without other specific immunostimulating agents, such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′ dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include MF59 (see WO 1990/014837 to Van Nest, G., et al., which is hereby incorporated by reference in its entirety), containing 5% Squalene, 0.5% TWEEN 80, and 0.5% Span 85 (optionally containing various amounts of MTP-PE) formulated into submicron particles using a microfluidizer; SAF, containing 10% Squalene, 0.4% TWEEN 80, 5% pluronic-blocked polymer L121, and thr-MDP, either microfluidized into a submicron emulsion or vortexed to generate a larger particle size emulsion; and the Ribi™ adjuvant system (RAS) (Ribi ImmunoChem, Hamilton, Mont.) containing 2% squalene, 0.2% TWEEN 80, and one or more bacterial cell wall components selected from the group consisting of monophosphoryllipid A (MPL), trehalose dimycolate (TDM), and cell wall skeleton (CWS), preferably MPL+CWS (Detox™). Other adjuvants include Complete Freund's Adjuvant (CFA), Incomplete Freund's Adjuvant (IFA), and cytokines, such as interleukins (IL-1, IL-2, and IL-12), macrophage colony stimulating factor (M-CSF), and tumor necrosis factor (TNF-α).

The choice of an adjuvant depends on the stability of the immunogenic formulation containing the adjuvant, the route of administration, the dosing schedule, the efficacy of the adjuvant for the species being immunized, and, in humans, a pharmaceutically acceptable adjuvant is one that has been approved or is approvable for human administration by pertinent regulatory bodies. For example, alum, MPL or Incomplete Freund's adjuvant (Chang, J. C. C., et al., 1998), which is hereby incorporated by reference in its entirety) alone or optionally all combinations thereof are suitable for human administration.

The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, non-immunogenic stabilizers, and the like.

Pharmaceutical compositions can also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Additionally, these carriers can function as immunostimulating agents (i.e., adjuvants).

The pharmaceutical compositions of the present invention can further include a suitable delivery vehicle. Suitable delivery vehicles include, but are not limited to viruses, bacteria, biodegradable microspheres, microparticles, nanoparticles, liposomes, collagen minipellets, and cochleates.

d. Methods of Using Pharmaceutical Compositions

The present disclosure also includes methods of using pharmaceutical compositions containing LAPP peptide immunogen constructs.

In certain embodiments, the pharmaceutical compositions containing IAPP peptide immunogen constructs can be used for the treatment of disorders associated with aggregated IAPP.

In some embodiments, the methods comprise administering a pharmaceutical composition comprising a pharmacologically effective amount of an IAPP peptide immunogen construct to a host in need thereof. In certain embodiments, the methods comprise administering a pharmaceutical composition comprising a pharmacologically effective amount of an IAPP peptide immunogen construct to a warm-blooded animal (e.g., humans, macaques, guinea pigs, mice, cat, etc.) to elicit highly specific antibodies cross-reactive with the aggregated human IAPP protein or IAPP proteins from other organisms (SEQ ID NOs: 3-7).

In certain embodiments, the pharmaceutical compositions containing IAPP peptide immunogen constructs can be used to treat disorders associated with aggregated IAPP in transgenic mice models.

e. In Vitro Functional Assays and In Vivo Proof of Concept Studies

Antibodies elicited in immunized hosts by the IAPP peptide immunogen constructs can be used in in vitro functional assays. These functional assays include, but are not limited to:
(1) in vitro binding to IAPP protein (SEQ ID NOs: 3-7) by serological assays including ELISA and dot blot assays;
(2) in vitro inhibition of aggregation of IAPP monomer or oligomer into IAPP fibril;
(3) in vitro inhibition of the cytotoxicity exerted by aggregated IAPP on RIN-m5F5 cells;
(4) reducing in vivo aggregated IAPP and disorders, such as T2D caused by aggregated IAPP in transgenic mice models.

### Specific Embodiments

(1) An IAPP peptide immunogen construct having about 20 or more amino acids, represented by the formulae:
(Th)ₘ-(A)ₙ-(IAPP functional B cell epitope peptide)-X
   or
(IAPP functional B cell epitope peptide)-(A)ₙ-(Th)ₘ-X
   or
(Th)ₘ-(A)ₙ-(IAPP functional B cell epitope peptide)-(A)ₙ-(Th)ₘ-X
wherein
Th is a heterologous T helper epitope;
A is a heterologous spacer;
(IAPP functional B cell epitope peptide) is a B cell epitope peptide having from 6 to about 28 amino acid residues of IAPP (SEQ ID NO: 3);
X is an α-COOH or α-CONH₂ of an amino acid;
m is from 1 to about 4; and
n is from 0 to about 10.

(2) The IAPP peptide immunogen construct according to (1), wherein the IAPP functional B cell epitope peptide is selected from the group consisting of SEQ ID NOs: 8-69.

(3) The IAPP peptide immunogen construct according to (1), wherein the Th epitope is selected from the group consisting of SEQ ID NOs: 73-112 and 171-182.

(4) The IAPP peptide immunogen construct according to (1), wherein the IAPP functional B cell epitope peptide is selected from the group consisting of SEQ ID NOs: 8-26 and the Th epitope is selected from the group consisting of SEQ ID NOs: 73-112 and 171-182.

(5) The IAPP peptide immunogen construct according to (1), wherein the peptide immunogen construct is selected from the group consisting of SEQ ID NOs: 113-167.

(6) An IAPP peptide immunogen construct comprising:
a. a B cell epitope comprising from about 6 to about 28 amino acid residues from the IAPP sequence of SEQ ID NOs: 3-7;
b. a T helper epitope comprising an amino acid sequence selected from the group consisting of SEQ ID NOs: 73-112 and 171-182, and any combination thereof; and
c. an optional heterologous spacer selected from the group consisting of an amino acid, Lys-, Gly-, Lys-Lys-Lys-, (α, ε-N)Lys, ε-N-Lys-Lys-Lys-Lys (SEQ ID NO: 71), Lys-Lys-Lys-ε-N-Lys (SEQ ID NO: 72), and Pro-Pro-Xaa-Pro-Xaa-Pro (SEQ ID NO: 70), and any combination thereof,
wherein the B cell epitope is covalently linked to the T helper epitope directly or through the optional heterologous spacer.

(7) The IAPP peptide immunogen construct of (6), wherein the B cell epitope is selected from the group consisting of SEQ ID NOs: 8-69.

(8) The IAPP peptide immunogen construct of (6), wherein the optional heterologous spacer is (α, ε-N)Lys, ε-N-Lys-Lys-Lys-Lys (SEQ ID NO: 71), Lys-Lys-Lys-ε-N-Lys (SEQ ID NO: 72), or Pro-Pro-Xaa-Pro-Xaa-Pro (SEQ ID NO: 70), where Xaa is any amino acid.

(9) The IAPP peptide immunogen construct of (6), wherein the T helper epitope is covalently linked to the amino- or carboxyl-terminus of the B cell epitope.

(10) The IAPP peptide immunogen construct of (6), wherein the T helper epitope is covalently linked to the amino- or carboxyl-of the B cell epitope through the optional heterologous spacer.

(11) A composition comprising the IAPP peptide immunogen construct according to (1).

(12) A pharmaceutical composition comprising:
a. a peptide immunogen construct according to (1); and
b. a pharmaceutically acceptable delivery vehicle and/or adjuvant.

(13) The pharmaceutical composition of (12), wherein
a. the IAPP functional B cell epitope peptide is selected from the group consisting of SEQ ID NOs: 8-69;
b. the Th epitope is selected from the group consisting of SEQ ID NOs: 73-112 and 171-182; and
c. the heterologous spacer is selected from the group consisting of an amino acid, Lys-, Gly-, Lys-Lys-Lys-, (α, ε-N)Lys, ε-N-Lys-Lys-Lys-Lys (SEQ ID NO: 71), Lys-Lys-Lys-ε-N-Lys (SEQ ID NO: 72), and Pro-Pro-Xaa-Pro-Xaa-Pro (SEQ ID NO: 70), and any combination thereof; and
wherein the IAPP peptide immunogen construct is mixed with an CpG oligodeoxynucleotide (ODN) to form a stabilized immunostimulatory complex.

(14) The pharmaceutical composition of (12), wherein
a. the LAPP peptide immunogen construct is selected from the group consisting of SEQ ID NOs: 113-139 and 140-167; and
wherein the IAPP peptide immunogen construct is mixed with an CpG oligodeoxynucleotide (ODN) to form a stabilized immunostimulatory complex.

(15) A method for generating antibodies against IAPP in an animal comprising administering the pharmaceutical composition according to (12) to the animal.

(16) An isolated antibody or epitope-binding fragment thereof that specifically binds to the amino acid sequence of SEQ ID NOs: 8-25.

(17) The isolated antibody or epitope-binding fragment thereof according to (16) bound to the IAPP peptide immunogen construct.

(18) A composition comprising the isolated antibody or epitope-binding fragment thereof according to (16).

(19) A method of preventing and/or treating disorders associated with aggregated IAPP. in an animal comprising administering the pharmaceutical composition of (12) to the animal.

### EXAMPLE 1

### Synthesis of IAPP Related Peptides and Preparation of Formulations Thereof

a. Synthesis of IAPP Related Peptides

Methods for synthesizing IAPP related peptides that were included in the development effort of IAPP peptide immunogen constructs are described. The peptides were synthesized in small-scale amounts that are useful for serological assays, laboratory pilot and field studies, as well as large-scale (kilogram) amounts, which are useful for industrial/commercial production of pharmaceutical compositions. A large repertoire of IAPP B cell epitope peptides having sequences with lengths from approximately 6 to 27 amino acids were designed for epitope mapping and for the screening and selection of the most optimal peptide immunogen constructs for use in an efficacious IAPP targeted therapeutic vaccine.

Table 1 and FIGS. 1A and 1C provide the full-length sequences of human IAPP and its precursors (SEQ ID NOs: 1-3), the full-length IAPP sequences from various organisms (SEQ ID NOs: 4-7 and 186-192), the sequences of IAPP peptide fragments (SEQ ID NOs: 8-26), and the sequences of 10-mer peptides employed for epitope mapping in various serological assays (SEQ ID NOs: 27-69.

Select LAPP B cell epitope peptides were made into LAPP peptide immunogen constructs by synthetically linking to a carefully designed helper T cell (Th) epitope peptide derived from pathogen proteins, including Measles Virus Fusion protein (MVF), Hepatitis B Surface Antigen protein (HBsAg), influenza, Clostridum tetani, and Epstein-Barr virus (EBV), identified in Table 2 (SEQ ID NOs: 73-112 and 171-182). The Th epitope peptides were used either in a single sequence (e.g., SEQ ID NOs: 73-83, 85-89, 91-92, 94-95, 97-174, 176-177, 179-182) or combinatorial library sequences (e.g., SEQ ID NOs: 84, 90, 93, 96, 175, and 178) to enhance the immunogenicity of their respective IAPP peptide immunogen constructs.

Representative IAPP peptide immunogen constructs selected from hundreds of peptide constructs are identified in Table 3 (SEQ ID NOs: 113-167). All peptides used for immunogenicity studies or related serological tests for detection and/or measurement of anti-IAPP antibodies were synthesized on a small-scale using F-moc chemistry by peptide synthesizers of Applied BioSystems Models 430A, 431 and/or 433. Each peptide was produced by an independent synthesis on a solid-phase support, with F-moc protection at the N-terminus and side chain protecting groups of trifunctional amino acids. Completed peptides were cleaved from the solid support and side chain protecting groups were removed by 90% Trifluoroacetic acid (TFA). Synthetic peptide preparations were evaluated by Matrix-Assisted Laser Desorption/Ionization-Time-Of-Flight (MALDI-TOF) Mass Spectrometry to ensure correct amino acid content. Each synthetic peptide was also evaluated by Reverse Phase HPLC (RP-HPLC) to confirm the synthesis profile and concentration of the preparation. Despite rigorous control of the synthesis process (including stepwise monitoring the coupling efficiency), peptide analogues were also produced due to unintended events during elongation cycles, including amino acid insertion, deletion, substitution, and premature termination. Thus, synthesized preparations typically included multiple peptide analogues along with the targeted peptide.

Despite the inclusion of such unintended peptide analogues, the resulting synthesized peptide preparations were nevertheless suitable for use in immunological applications including immunodiagnosis (as antibody capture antigens) and pharmaceutical compositions (as peptide immunogens). Typically, such peptide analogues, either intentionally designed or generated through synthetic process as a mixture of byproducts, are frequently as effective as a purified preparation of the desired peptide, as long as a discerning QC procedure is developed to monitor both the manufacturing process and the product evaluation process to guarantee the reproducibility and efficacy of the final product employing these peptides. Large scale peptide syntheses in the multi-hundred to kilo gram quantities can be conducted on a customized automated peptide synthesizer UBI2003 or the like at 15 mmole to 150 mmole scale.

For active ingredients used in the final pharmaceutical composition for clinical trials, IAPP peptide immunogen constructs were purified by preparative RP-HPLC under a shallow elution gradient and characterized by MALDI-TOF mass spectrometry, amino acid analysis and RP-HPLC for purity and identity.

b. Preparation of Compositions Containing IAPP Peptide Immunogen Constructs

Formulations employing water-in-oil emulsions and in suspension with mineral salts were prepared. In order for a pharmaceutical composition designed to be used by a large population, safety becomes another important factor for consideration. Despite the fact that water-in-oil emulsions have been used in humans as pharmaceutical compositions in many clinical trials, Alum remains the major adjuvant for use in pharmaceutical composition due to its safety. Alum or its mineral salts ADJUPHOS (Aluminum phosphate) are therefore frequently used as adjuvants in preparation for clinical applications.

Briefly, the formulations specified in each of the study groups described below generally contained all types of designer IAPP peptide immunogen constructs. A multitude of designer IAPP peptide immunogen constructs were carefully evaluated in guinea pigs for their relative immunogenicity against the corresponding IAPP peptide used as the B cell epitope peptide. Epitope mapping and serological cross-reactivities were analyzed amongst the varying homologous peptides by ELISA assays using plates coated with peptides selected from the list with SEQ ID NOs: 3-69.

The IAPP peptide immunogen constructs at varying amounts were prepared in a water-in-oil emulsion with SEPPIC MONTANIDE™ ISA 51 as the approved oil for human use, or mixed with mineral salts ADJUPHOS (Aluminum phosphate) or ALHYDROGEL (Alum) as specified. Compositions were typically prepared by dissolving the IAPP peptide immunogen constructs in water at about 20 to 2,000 μg/mL and formulated with MONTANIDE™ ISA 51 into water-in-oil emulsions (1:1 in volume) or with mineral salts ADJUPHOS or ALHYDROGEL (Alum) (1:1 in volume). The compositions were kept at room temperature for about 30 min and mixed by vortex for about 10 to 15 seconds prior to immunization. Animals were immunized with 2 to 3 doses of a specific composition, which were administered at time 0 (prime) and 3 week post initial immunization (wpi) (boost), optionally 5 or 6 wpi for a second boost, by intramuscular route. Sera from the immunized animals were then tested with selected B cell epitope peptide(s) to evaluate the immunogenicity of the various IAPP peptide immunogen constructs present in the formulation and for the corresponding sera's cross-reactivity with the full-length oligomeric or aggregated IAPP with IAPP proteins. Those IAPP peptide immunogen constructs with potent immunogenicity found in the initial screening in guinea pigs were further tested in in vitro assays for their corresponding sera's functional properties. The selected candidate IAPP peptide immunogen constructs were then prepared in water-in-oil emulsion, mineral salts, and alum-based formulations for dosing regimens over a specified period as dictated by the immunization protocols.

Only the most promising LAPP peptide immunogen constructs were further assessed extensively prior to being incorporated into final formulations for immunogenicity, duration, toxicity and efficacy studies in GLP guided preclinical studies in preparation for submission of an Investigational New Drug application followed by clinical trials in patients suffering from disorders associated with aggregated IAPP.

The following examples serve to illustrate the present invention and are not to be used to limit the scope of the invention.

### EXAMPLE 2

### Serological Assays and Reagents

Serological assays and reagents for evaluating functional immunogenicity of the IAPP peptide immunogen constructs and formulations thereof are described in detail below.

a. IAPP or IAPP B Cell Epitope Peptide-Based ELISA Tests for Immunogenicity and Antibody Specificity Analysis

ELISA assays for evaluating immune serum samples described in the following Examples were developed and described below. The wells of 96-well plates were coated individually for 1 hour at 37° C. with 100 μL of IAPP or IAPP B cell epitope peptides (e.g., SEQ ID NOs: 3 to 69), at 2 μg/mL (unless noted otherwise), in 10 mM NaHCO₃ buffer, pH 9.5 (unless noted otherwise).

The IAPP or IAPP B cell epitope peptide-coated wells were incubated with 250 μL of 3% by weight gelatin in PBS at 37° C. for 1 hour to block non-specific protein binding sites, followed by three washes with PBS containing 0.05% by volume TWEEN® 20 and dried. Sera to be analyzed were diluted 1:20 (unless noted otherwise) with PBS containing 20% by volume normal goat serum, 1% by weight gelatin and 0.05% by volume TWEEN® 20. One hundred microliters (100 μL) of the diluted specimens (e.g., serum, plasma) were added to each of the wells and allowed to react for 60 minutes at 37° C. The wells were then washed six times with 0.05% by volume TWEEN® 20 in PBS in order to remove unbound antibodies. Horseradish peroxidase (HRP)-conjugated species (e.g., guinea pig or rat) specific goat polyclonal anti-IgG antibody or Protein A/G were used as a labeled tracer to bind with the antibody/peptide antigen complex formed in positive wells. One hundred microliters (100 μL) of the HRP-labeled detection reagent, at a pre-titered optimal dilution and in 1% by volume normal goat serum with 0.05% by volume TWEEN® 20 in PBS, was added to each well and incubated at 37° C. for another 30 minutes. The wells were washed six times with 0.05% by volume TWEEN® 20 in PBS to remove unbound antibody and reacted with 100 μL of the substrate mixture containing 0.04% by weight 3′, 3′, 5′, 5′-Tetramethylbenzidine (TMB) and 0.12% by volume hydrogen peroxide in sodium citrate buffer for another 15 minutes. This substrate mixture was used to detect the peroxidase label by forming a colored product. Reactions were stopped by the addition of 100 μL of 1.0M H₂SO₄ and absorbance at 450 nm (A₄₅₀) determined. For the determination of antibody titers of the vaccinated animals that received the various peptide vaccine formulations, a 10-fold serial dilutions of sera from 1:100 to 1:10,000 or a 4-fold serial dilutions of sera from 1:100 to 1:4.19×10⁸ were tested, and the titer of a tested serum, expressed as Logio, was calculated by linear regression analysis of the A₄₅₀ with the cutoff A₄₅₀ set at 0.5.

b. Dot Blot Tests Employing Full Length Monomeric, Oligomeric or Aggregated IAPP Molecule for Immunogenicity and Antibody Specificity Analysis

PVDF membrane and Bio-Dot Apparatus (Bio-Rad) were used in this experiment. The PVDF membrane was assembled into the device then wash with methanol. Two hundred microliters (200 μL) of TBST (TBS buffer with 0.1% TWEEN® 20) was loaded into each well twice to wash away remaining methanol. One hundred nanograms (100 ng) of target protein or peptide (e.g. monomeric, oligomeric, or aggregated IAPP) was applied to the wells and trapped by suction. The IAPP coated wells were incubated with 200 μL of 10% skim milk in TBS at 37° C. for 1 hour to block non-specific protein binding, followed by three washes with TBS containing 0.05% TWEEN® 20 and dried. One hundred microliters (100 μL) of diluted sera samples from immunized guinea pigs were added to each well and allowed to react for 2 hours at 37° C. The wells were then washed with TBS containing 0.05% TWEEN® 20 five times. The apparatus was opened to obtain the PVDF membrane. The membrane was then incubated with peroxidase-labeled rabbit anti-guinea pig IgG with optimal dilution prepared in TBS containing 5% skim milk with the reagent added to each well and incubated at 37° C. for 1 hour. After the incubation, wells were washed with TBS containing 0.05% TWEEN® 20 three times and membrane reacted with chemiluminescent substrate. The reactions were detected by UVP BioDoc-It 220 Imaging System.

c. Assessment of Antibody Reactivity Towards Th Peptide by Th Peptide-Based ELISA Tests

The wells of 96-well ELISA plates were coated individually for 1 hour at 37° C. with 100 μL of Th peptide at 2 μg/mL (unless noted otherwise), in 10 mM NaHCO₃ buffer, pH 9.5 (unless noted otherwise) in similar ELISA method and performed as described above. For the determination of antibody titers of the vaccinated animals that received the various formulations containing LAPP peptide immunogen constructs, 10-fold serial dilutions of sera from 1:100 to 1:10,000 were tested, and the titer of a tested serum, expressed as Log₁₀, was calculated by linear regression analysis of the A₄₅₀ with the cutoff A₄₅₀ set at 0.5.

d. Fine Specificity Analyses of a Target IAPP B Cell Epitope Peptide Determined by Epitope Mapping Through B Cell Epitope Cluster 10-Mer Peptide-Based ELISA Tests

Fine specificity analyses of anti-IAPP antibodies from hosts immunized with IAPP peptide immunogen constructs were determined by epitope mapping using B cell epitope cluster 10-mer peptide-based ELISA tests. Briefly, the wells of 96-well plates were coated with individual IAPP or related 10-mer peptides (SEQ ID NOs: 26-69) at 0.5 μg per 0.1 mL per well and then 100 μL serum samples (1:100 dilution in PBS) were incubated in 10-mer plate wells in duplicate following the steps of the antibody ELISA method described above. The target B cell epitope related fine specificity analyses of anti-IAPP antibodies from immunized hosts were tested with the corresponding IAPP peptide, or with non-relevant control peptide for specificity confirmation.

e. Immunogenicity Evaluation

Preimmune and immune serum samples from animal subjects were collected according to experimental vaccination protocols and heated at 56° C. for 30 minutes to inactivate serum complement factors. Following the administration of the formulations containing the IAPP peptide immunogen constructs, blood samples were obtained according to protocols and their immunogenicity against specific target site(s) were evaluated using the corresponding IAPP B cell epitope peptide-based ELISA tests. Serially diluted sera were tested and positive titers were expressed as Logio of the reciprocal dilution. Immunogenicity of a particular formulation was assessed for its ability to elicit high titer antibody response directed against the desired epitope specificity within the target antigen and high cross-reactivities with the IAPP polypeptide, while maintaining a low to negligible antibody reactivity towards the helper T cell epitopes employed to provide enhancement of the desired B cell responses.

### EXAMPLE 3

### Assessment of Functional Properties of Antibodies Elicted by the IAPP Peptide Immunnogen Constructs and Formulations Thereof

Immune sera or purified anti-IAPP antibodies in guinea pigs were further tested for their ability to (1) bind to monomer, oligomer, and fibril forms of IAPP; (2) suppress the IAPP amyloid fibrillation; and (3) inhibit the cytotoxic effects of hIAPP aggregates in RIN-m5F cells.

a. IAPP Oligomer Production

Highly purified monomeric IAPP was dissolved in 1,1,1,3,3,3-Hexafluoro-2-propanol (HFIP) (1 mg/mL) and incubate at 37° C. for 2 hours. The solvent HFIP was thereafter removed by SpeedVac apparatus with the peptide being resuspended in DMSO at a concentration of 20 μg/μL followed by further dilution with phosphate-buffered saline (pH 7.4) containing 1% SDS to a final concentration of 100 μM.

After incubation at 37° C. overnight, dialysis was performed. Samples were loaded into Slide-A-Lyzer™ Dialysis Cassettes 10K MWCO (Thermo) and dialyzed with 1% SDS/PBS for 24 hr at room temperature. SDS concentration was serially diluted 2-fold by changing buffer once every 24 hours until the SDS concentration was below 0.01%.

b. Dot Blot Assay

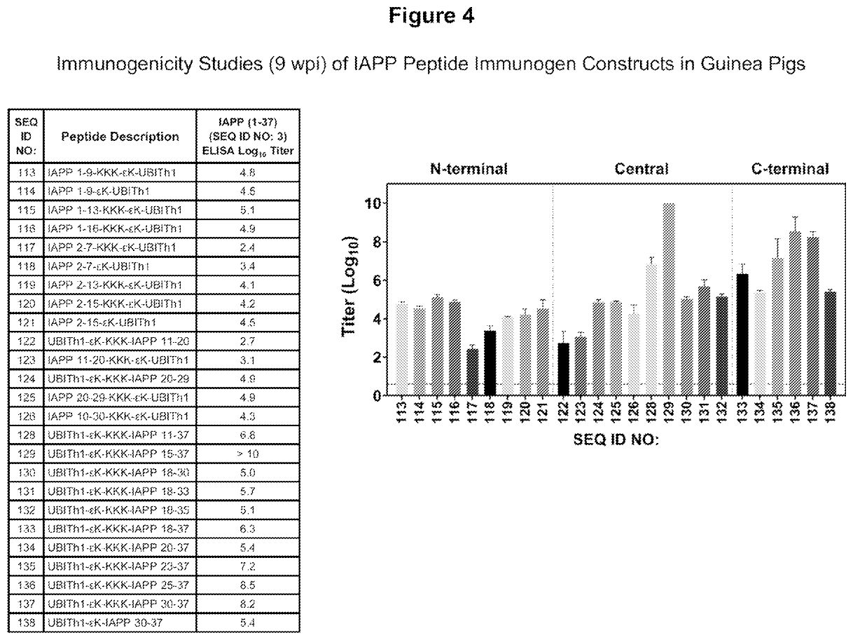

Dot blot tests employing full-length monomeric, oligomeric, or aggregated IAPP polypeptide for immunogenicity and antibody specificity analysis were performed as described in Example 2 above and the results are shown in FIGS. 4, 5, and 6.

c. Thioflavin T (ThT) Assay

ThT (Sigma) was dissolved in PBS (pH 7.4) and diluted to 10 mM as a stock solution and was then further diluted to 30 μM as a working solution. IAPP oligomer and monomer were prepared as described above. The oligomers or monomers were dissolved in DMSO then diluted with PBS to a final concentration of 30 μM. Purified IgGs (100 μg/mL) from guinea pig immune sera were added to the solution and incubated at 37° C. Thereafter, the solutions were mixed with ThT at a 1:1 ratio to test for RFU (Relative Fluorescence Units) which was read with excitation at 440 nm and emission at 485 nm, at both 0 and 24 hours.

d. Cells

RIN-m5F cell line was purchased from the American Type Culture Collection (Manassas, VA) and maintained in DMEM medium supplemented with 10% Fetal Bovine Serum (FBS), 4.5 g/L L-glutamine, sodium pyruvate, and 1% penicillin/streptomycin in a humidified 37° C. incubator with 5% CO₂.

e. MTT Cell Viability Assay

RIN-m5F cells (20,000 cells/well) were cultured in 96-well microplates (100 μL/well) and incubated overnight at 37° C. Human oligomers (5 μM total peptide) were added to each well in the presence of the antibodies at various concentrations. IAPP monomer/oligomer preparations were prepared as described above. The IAPP monomer, oligomer, or aggregated fibril preparation was dissolved with DMSO then diluted with PBS to a final concentration of 40 μM. The IAPP preparations were then incubated with serially diluted preparations of purified guinea pig polyclonal antibodies against IAPP peptide immunogen constructs before conducting the assay to assess the ability of the antibodies to neutralize cell death from the IAPP preparations. After incubation, the IAPP preparation/antibody mixtures were added to the wells containing the RIN-m5F cells for 24 hours.

MTT (3-(4,5-cimethylthiazol-2-yl)-2,5-diphenyl tetrazolium bromide) was dissolved in PBS (5 mg/mL) before treating cells. Medium was removed from each well before adding 110 μL of MTT/medium mixture (10% MTT). The cells were then incubated for 4 hours at 37° C. Eighty microliters (80 μL) of DMSO were added to each well and the cells were incubated for another 10 minutes at 37° C. after removing the mixture. Color intensity was measured at 540 nm.

Rin-m cells (2×10⁵ cells/mL) were cultured in 96-well microplates (100 μL/well) and incubated overnight at 37° C. Human oligomers (5 μM, total peptide) were added to each well in the presence of the antibodies at various concentrations. Each measurement was repeated four times. A control measurement with antibodies alone at the highest concentration was performed to evaluate any effect of the antibodies on cell viability. Following incubation for 6 hours at 37° C., cell viability was evaluated using the MTT assay.

### EXAMPLE 4

### Animals Used in Safety, Immunogenicity, Toxicity, and Efficacy Studies

a. Guinea Pigs:

Immunogenicity studies were conducted in mature, naïve, adult male and female Duncan-Hartley guinea pigs (300-350 g/BW). The experiments utilized at least 3 Guinea pigs per group.

Protocols involving Duncan-Hartley guinea pigs (8-12 weeks of age; Covance Research Laboratories, Denver, PA, USA) were performed under approved IACUC applications at a contracted animal facility under UBI sponsorship.

b. Cynomolgus Macaques:

Immunogenicity and repeated dose toxicity studies in adult male and female monkeys (Macaca fascicularis, approximately 3-4 years of age; Joinn Laboratories, Suzhou, China) were conducted under approved IACUC applications at a contracted animal facility under UBI sponsorship.

c. Mice

Representative IAPP peptide immunogen constructs were validated in a transgenic mice model expressing hIAPP: FVB/hIAPP (hemizygous) RHFSoel/J mice exposed to high-fat and high-sucrose diet (HFFD). The preventive and/or therapeutic efficacy was assessed by determining the β cell mass and hIAPP amyloid load in the pancreas as well as plasma levels of hIAPP along with functional tests of glucose metabolism and insulin secretion.

### EXAMPLE 5

### Formulations for Immunogenicity Assessment of IAPP Peptide Immunogen Constructs in Guinea Pigs

Pharmaceutical formulations comprising LAPP peptide immunogen constructs were prepared. Briefly, the formulations specified in each of the study groups generally contained all types of designer LAPP peptide immunogen constructs with a segment of the LAPP B cell epitope peptide linked via different type of spacers (e.g., εLys (εK) and/or lysine-lysine-lysine (KKK) to enhance the solubility of the peptide construct) and promiscuous Th epitopes, including two sets of artificial Th epitopes derived from Measles virus fusion protein and Hepatitis B surface antigen. The IAPP B cell epitope peptides were linked at the N- or C-terminus of the designer peptide constructs. A multitude of designer IAPP peptide immunogen constructs were initially evaluated in guinea pigs for their relative immunogenicity against the corresponding IAPP B cell epitope peptides. Various amounts of IAPP peptide immunogen constructs were prepared in a water-in-oil emulsion with SEPPIC MONTANIDE ISA 51 as the approved oil for human vaccine use, or with mineral salts (ADJUPHOS) or ALHYDROGEL (Alum) as a suspension, as specified. Formulations were usually prepared by dissolving the IAPP peptide immunogen constructs in water at about 20 to 800 μg/mL and formulated either with MONTANIDE ISA 51 into water-in-oil emulsions (1:1 in volume) or with mineral salts (ADJUPHOS) or ALHYDROGEL (Alum) (1:1 in volume). The formulations were kept at room temperature for about 30 min and mixed by vortex for about 10 to 15 seconds prior to immunization.

Some animals were immunized with 2 to 5 doses of a specific formulation, which were administered at time 0 (prime) and 3 weeks post initial immunization (wpi) (boost), and optionally at 5 wpi or 6 wpi for a second boost, by intramuscular route. The sera from immunized animals were then evaluated for the immunogenicity against either the corresponding IAPP peptide immunogen constructs used in the respective formulation, the full-length oligomeric or aggregated IAPP with the corresponding IAPP B cell epitope peptides, or full-length IAPP. The IAPP peptide immunogen constructs with potent immunogenicity in the initial screening in guinea pigs were further tested in both water-in-oil emulsion, mineral salts, and alum-based formulations in macaques for dosing regimens over a specified period as dictated by the immunization protocols.

The IAPP peptide immunogen constructs that were highly immunogenic were further evaluated for their ability to breakout immune tolerance in mice using corresponding mouse IAPP peptide immunogen constructs. The IAPP peptide immunogen constructs with best immunogenicity in mice elicited anti-IAPP antibody titers against endogenous IAPP.

Optimized IAPP peptide immunogen constructs can be incorporated into final formulations for GLP guided immunogenicity, duration, toxicity, and proof of efficacy studies in preparation for submission of an Investigational New Drug Application and clinical trials in patients with disorders associated with aggregated IAPP.

### EXAMPLE 6

### Design Rationale, Screening, Identification, Assessment of Functional Properties, and Optimization of Multi-Component Vaccine Formulations Incorporating IAPP Peptide Immunogen Constructs for Treatment of Disorders Associated with Aggregated IAPP

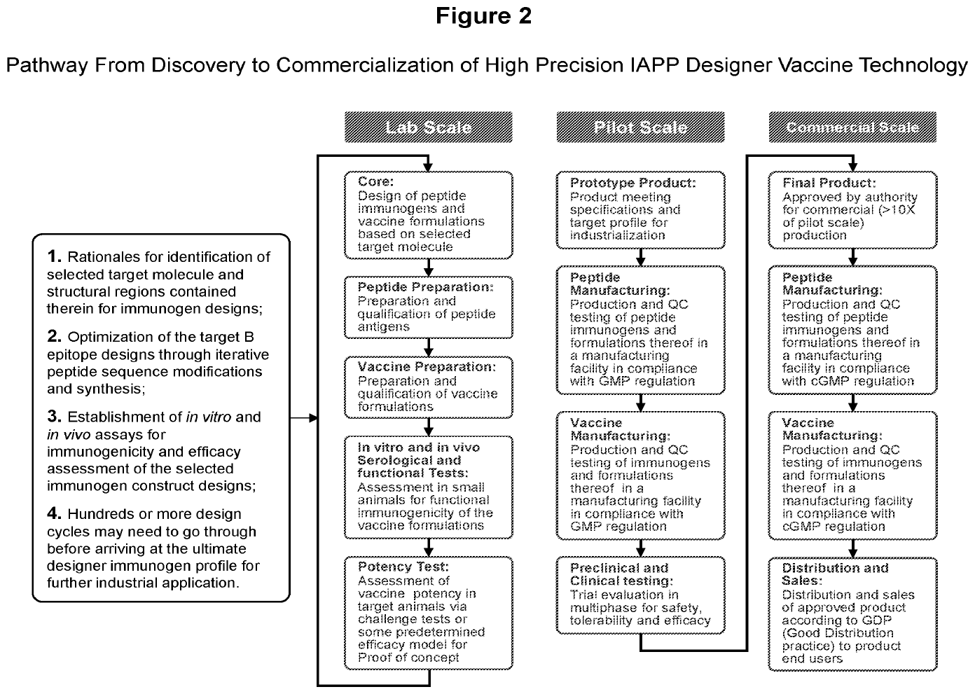

Based on scientific information described in the background section, IAPP was selected as the target polypeptide for design of the disclosed peptide immunogen constructs. FIG. 1C presents the sequence alignment of IAPP sequences from human (SEQ ID NO: 3), cat (SEQ ID NO: 4), macaque (SEQ ID NO: 5) and several other organisms. In addition, the general features and physiology associated with IAPP have been described by others (e.g., Hayden, M. R., et al., 2001 and Hay, D. L., et al., 2015). FIG. 2 depicts the pathways from discovery to commercialization (industrialization) of high precision designer IAPP peptide immunogen constructs. Detailed evaluation and analyses of each of the steps has led to a myriad of experiments in the past which would ultimately result in commercialization of a safe and efficacious pharmaceutical formulation containing IAPP peptide immunogen constructs.

a. Design History

Each peptide immunogen construct or immunotherapeutic product requires its own design focus and approach based on its specific disease mechanism and the target protein(s) required for intervention. For treatment of disorders associated with aggregated IAPP, IAPP was selected as the target molecule based on the scientific information available as outlined in the background and FIGS. 1A-1C. The pathway from discovery to commercialization as shown in FIG. 2 typically requires one or more decades to accomplish. Identification of the IAPP B cell epitope peptides correlating to the functional site(s) for intervention are key to the immunogen construct design. Consecutive pilot immunogenicity studies in guinea pigs incorporating various T helper support (carrier proteins or suitable T helper epitope peptides) in various formulations were conducted and subsequently evaluated for the functional properties of the elicited purified antibodies or the formulations employing specific IAPP peptide immunogen constructs in several in vitro functional assays or proof of concept in vivo studies in a selected animal model. Upon extensive serological validation, candidate IAPP B cell epitope peptide immunogen constructs can be further tested in non-human primates to further validate the immunogenicity and direction of the IAPP peptide immunogen design. Selected IAPP peptide immunogen constructs can be prepared in varying mixtures to evaluate the subtle differences in functional properties related to the respective interactions amongst peptide constructs when used in combinations. Upon additional evaluation, the final peptide constructs, peptide compositions, and formulations thereof, along with the respective physical parameters of the formulations can be established, leading to the final product development process.

The IAPP peptide immunogen constructs of the present disclosure were designed and selected based on a number of rationales, including:
(i) the IAPP B cell epitope peptide is non-immunogenic on its own to avoid autologous T cell activation;
(ii) the IAPP B cell epitope peptide can be rendered immunogenic by using a protein carrier or a potent T helper epitope(s);
(iii) when the IAPP B cell epitope peptide is rendered immunogenic and administered to a host, the peptide immunogen construct:
a. elicits high titer antibodies preferentially directed against the IAPP B cell epitope(s) and not the protein carrier or T helper epitope(s);
b. breaks immune tolerance in the immunized host and generates highly specific antibodies having cross-reactivity with the full-length oligomeric or aggregated IAPP (e.g., SEQ ID NOs: 3-7);
c. generates highly specific antibodies capable of inhibiting aggregation of IAPP monomer or oligomer into IAPP fibril;
d. generates highly specific antibodies capable of inhibiting the associated cytotoxicity exerted by the aggregated IAPP to β cells; and
e. generates highly specific antibodies capable of reducing in vivo aggregated IAPP; and
f. is capable of treating and/or preventing disorders caused by aggregated IAPP.

The disclosed IAPP peptide immunogen constructs and formulations thereof can effectively function as a pharmaceutical composition or vaccine formulation to prevent and/or treat subjects predisposed to, or suffering from, disorders related to aggregated IAPP.

b. Design and Validation of IAPP Peptide Immunogen Constructs for Pharmaceutical Compositions with the Potential to Treat Patients Suffering from Disorders Associated with Aggregated IAPP.

In order to generate the most potent peptide constructs for incorporation into the pharmaceutical compositions, a repertoire of human IAPP B cell epitope peptides (e.g., SEQ ID NOs: 8-69) and promiscuous T helper epitopes derived from various pathogens or artificially T helper epitopes (e.g. SEQ ID NOs: 73-112 and 171-182) were further designed and made into representative IAPP peptide immunogen constructs (e.g., SEQ ID NOs: 113-167) for immunogenicity studies initially in guinea pigs.

i) Selection of IAPP B Cell Epitope Peptide Sequences from the Sites that are (a) Prone to IAPP Aggregation; (b) Responsible for Interaction of the IAPP Polypeptide with the Membrane; and (c) Responsible for the Cytotoxicity of β Cells.

The IAPP B cell epitope peptide sequences were selected for IAPP B cell epitope design. Peptide immunogen constructs were then prepared with these B cell epitopes to elicit antibodies in guinea pigs, which were initially evaluated for immunogenicity by ELISA using the respective B cell epitope peptide or full-length IAPP, and subsequently used for in vitro functional assay assessment.

IAPP peptide immunogen constructs (SEQ ID NOs: 113-138, containing the B cell epitopes of SEQ ID NOs: 8-26) were formulated initially with ISA 51 and CpG for prime immunization in guinea pigs at 400 μg/1 mL and boosts (given at 3, 6, and 9 wpi) at 100 μg/0.25 mL for immunogenicity studies. These peptide immunogen constructs contained B cell epitopes from the N-terminal (SEQ ID NOs: 113-121), Central (SEQ ID NOs: 122-132), and C-terminal (SEQ ID NOs: 133-138) regions of IAPP.

To test the immunogenicity of the IAPP peptide immunogen constructs in guinea pigs, ELISA assay was used with guinea pig immune sera from various bleeds (0, 3, 6, 9, 12, and 15 wpi), diluted at a 10-fold serial dilution from 1:100 to 1:10,000. ELISA plates were coated with full-length human IAPP peptide (SEQ ID NO: 3) at 0.5 μg peptide per well. The titer of a tested serum, expressed as Log₁₀, was calculated by linear regression analysis of the A450 nm with the cutoff A₄₅₀ set at 0.5.

Table 4 provides the immunogenicity results obtained for each immunized animal at 0, 3, 6, 9, 12, and 15 wpi. FIG. 4 summarizes the average relative immunogenicity profile for each LAPP peptide immunogen construct (SEQ ID NOs: 113-138) from guinea pig sera at 9 wpi. As can be easily observed, the peptide immunogen constructs derived from, or containing, the C-terminal region of IAPP have a relatively higher immunogenicity compared to peptide immunogen constructs derived from other regions of the IAPP polypeptide. The peptide immunogen construct of SEQ ID NO: 129, containing the IAPP B cell epitope with amino acids from 15-37 (SEQ ID NO: 18), had the highest immunogenicity.

ii) Autologous T Helper Epitopes are not Present within Selected IAPP B Cell Epitopes

Generally, and ideally, short B cell epitope peptides are non-immunogenic on their own, due to the lack of endogenous Th epitopes. Short B cell epitope peptides that are highly immunogenic on their own suggest that a Th epitope is present within the amino acid sequence. Thus, an experiment was performed on select short IAPP B cell epitope peptides to determine if they are capable of eliciting an immune response on their own.

Specifically, the short IAPP B cell epitope peptides containing aa1-13 (SEQ ID NO: 9) and aa1-16 (SEQ ID NO: 10) were evaluated. Immunizing guinea pigs with formulations containing these short B cell epitope peptides found that these peptides were non-immunogenic, as shown in Table 5. However, these short B cell epitopes were able to elicit an appreciable immune response when presented in a peptide immunogen construct containing a foreign Th epitope (UBITH®1; SEQ ID NO: 97). Specifically, SEQ ID NOs: 115 and 116, which contained the B cell epitopes of SEQ ID NOs: 9 and 10, respectively, became immunogenic, when presented as peptide immunogen constructs, as shown in Table 4. Therefore, the use of foreign Th epitopes enhanced the immunogenicity of the short, non-immunogenic B cell epitope peptides.

iii) The Antibody Response Elicited by IAPP Peptide Immunogen Constructs is Targeted at the IAPP B Cell Epitope Only

It is well known that carrier proteins (e.g., Keyhole Limpet Hemocyanin (KLH), Diphtheria toxoid (DT) and Tetanus Toxoid (TT) proteins) used to potentiate an immune response against targeted B cell epitope peptides, by chemical conjugation of such B cell epitope peptides to the respective carrier protein, will elicit more than 90% of the antibodies against the potentiating carrier protein with less than 10% of the antibodies directed against the targeted B cell epitope in immunized hosts.

It is therefore of interest to assess the specificity of the disclosed IAPP peptide immunogen constructs, to confirm that the antibodies are directed against the B cell epitope peptides and not the Th epitopes. Two representative LAPP peptide immunogen constructs (SEQ ID NOs: 137 and 136), with B cell epitopes from LAPP aa30-37 (SEQ ID NO: 26) and aa25-37 (SEQ ID NO: 25), respectively, linked through a spacer to the heterologous T cell epitope UBITh®1 (SEQ ID NO: 97), were prepared for immunogenicity assessment. Sera from guinea pigs immunized with SEQ ID NOs: 137 and 136 were evaluated for their immunogenicity against the UBITh®1 peptide by ELISA assay to test for their cross-reactivities with the UBITh®1 peptide used for immunopotentiation. In this experiment, it was found that most, if not all, of the immune sera were found non-reactive to the UBITh®1 peptide as shown in Table 6. These results are in contrast with the high immunogenicities of these constructs towards their corresponding targeted LAPP B cell epitope peptides, as illustrated by the high titers of antibodies (˜5 Log₁₀) generated towards the IAPP B cell epitope(s) upon even a single shot (SEQ ID NOs: 136 and 137, as shown in Table 4).

In summary, simple immunogen design incorporating target IAPP B cell epitope peptide linked to carefully selected T helper epitopes allows for the generation of a focused immune response targeted only to the corresponding IAPP B cell epitope peptide. For pharmaceutical composition design, the more specific the immune response a peptide immunogen generates, the higher safety profile it provides for the composition. Therefore, the IAPP peptide immunogen constructs of the present disclosure is highly specific and highly potent against its B cell target, suggesting that they are also very safe.

iv) Fine Epitope Mapping with Immune Sera Directed Against Selected IAPP Peptide Immunogen Constructs

A fine epitope mapping study was performed to localize the antibody binding site(s) to specific residues within the IAPP polypeptide. Specifically, 43 overlapping 10-mer peptides (SEQ ID NOs: 27-69) were synthesized that cover the IAPP polypeptide from amino acid −7 to amino acid 44, covering the full-length region of IAPP along with the precursor sequences before and after the processed IAPP polypeptide. These 10-mer peptides were individually coated onto 96-well microtiter plate wells as solid-phase immune-absorbents. Guinea pig antisera for multiple peptide immunogen constructs (SEQ ID NOs: 118, 113, 116, 123, 125, 128, 129, 133, 134, 135, 136, and 137) were added at a 1:100 dilution in specimen diluent buffer to ELISA plate wells coated with 10-mer peptide at 2.0 μg/mL followed by incubation for one hour at 37° C. After washing the wells of the plate with wash buffer, horseradish peroxidase-conjugated rProtein A/G was added and incubated for 30 min. After washing with PBS, substrate was added to the wells for measurement of absorbance at 450 nm by ELISA plate reader. The samples were analyzed in duplicate. The binding of antibodies from immune sera obtained from animals immunized with IAPP peptide immunogen constructs was evaluated against the corresponding IAPP B cell epitope peptide coated wells to determine the maximal antibody binding signal.

The results from this fine epitope mapping experiment are shown in Table 7. A summary of the results is as follows:
a. Peptide immunogen constructs derived from, or containing, the C-terminal region of IAPP (SEQ ID NOs: 128, 129, and 133-137) elicited antibodies with high reactivities against the full-length IAPP peptide (SEQ ID NO: 3). All of these constructs elicited strong antibodies directed toward 10-mer peptides from aa28-37 of IAPP. The IAPP peptide immunogen constructs of SEQ ID NOs: 128 (aa11-37), 129 (aa15-37), 134 (aa20-37) and 135 (aa23-37) were not found to be reactive against other B cell epitope regions of IAPP.
b. Peptide immunogen constructs derived from, or containing, the Central region of IAPP (SEQ ID NOs: 123, 125, 128 and 129) had weak to moderate reactivities against the full-length IAPP polypeptide. The peptide immunogen construct of SEQ ID NO: 125 moderately reacted with B cell epitopes spanning aa18-29, while the peptide immunogen construct of SEQ ID NO: 123 exclusively reacted with the B cell epitope peptide covering aa11-20, which is the central membrane binding alpha helical region of IAPP.
c. Peptide immunogen constructs derived from, or containing, B cell epitope peptides covering the N-terminal region of IAPP (SEQ ID NOs: 118, 113, and 116) had relatively weak reactivities against the full-length IAPP polypeptide.

In summary, the designed synthetic IAPP peptide immunogen constructs with B cell epitopes derived from, or containing, the C-terminal region of IAPP induced robust immune responses in guinea pigs that generated polyclonal antibodies targeted against distinct clusters of 10-mer peptides within IAPP. The IAPP peptide immunogen construct of SEQ ID NO: 133 (containing a B cell epitope with aa18-37) had a focused reactivity towards aa18-29, which is near the IAPP aggregation prone region. The epitope mapping study, along with additional functional assay assessment, can provide for the identification of the most optimal peptide immunogen constructs formulations.

### v) IAPP Monomer Binding Profiles by Dot Blot Binding Assay

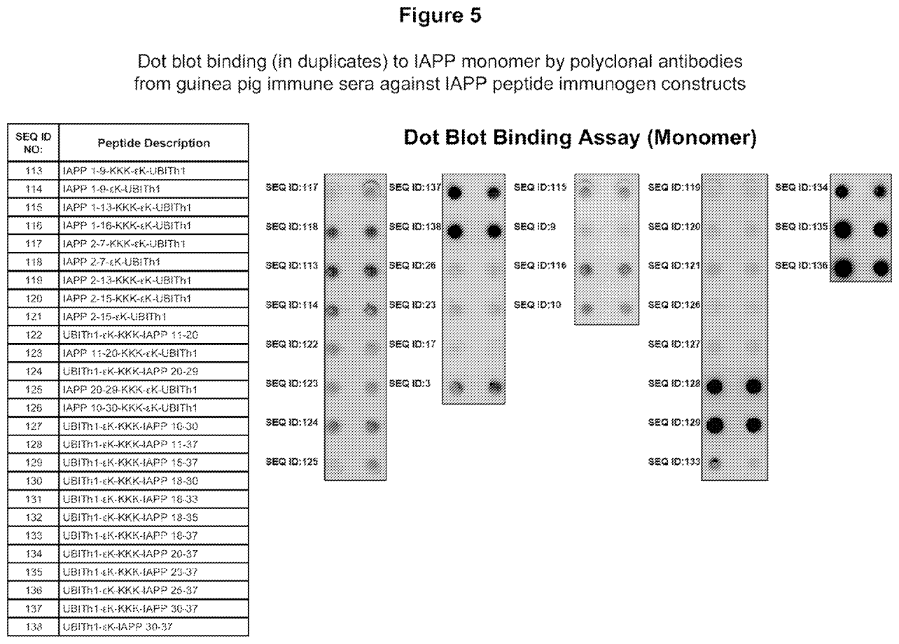

Antibodies elicited by the peptide immunogen constructs of SEQ ID NOs: 113-138 were evaluated for their abilities to bind to IAPP monomers. FIG. 5 depicts the IAPP monomer binding profiles by dot blot binding assays with guinea pig immune sera collected at 12 wpi (all samples were run in duplicates). The results demonstrate that the peptide immunogen constructs derived from, or containing, the C-terminal region of IAPP (e.g., SEQ ID NOs: 128, 129, and 134-138) had the strongest binding profile to the IAPP monomer compared to the other peptide immunogen constructs.

### vi) IAPP Oligomer Binding Profiles by Dot Blot Binding Assay

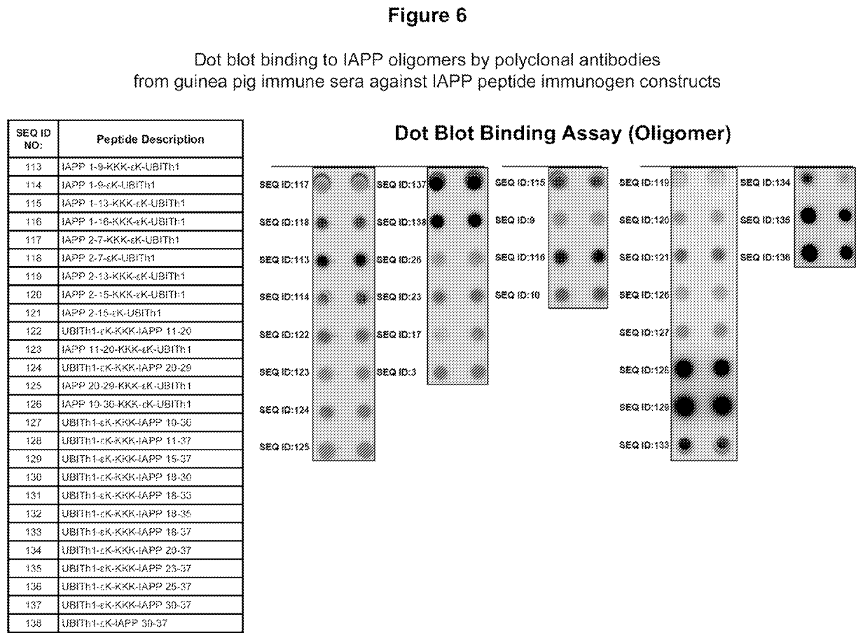

Antibodies elicited by the peptide immunogen constructs of SEQ ID NOs: 113-138 were evaluated for their abilities to bind to IAPP oligomers. FIG. 6 depicts the IAPP oligomer binding profiles by dot blot binding assay with guinea pig immune sera collected at 12 wpi (all samples run in duplicate). The results demonstrate that the peptide immunogen constructs derived from, or containing, the C-terminal region of IAPP (e.g., SEQ ID NOs: 128, 129, and 134-138) had the strongest binding profile to the IAPP oligomer compared to the other peptide immunogen constructs.

vii) IAPP Fibril Binding Profiles by Dot Blot Binding Assay

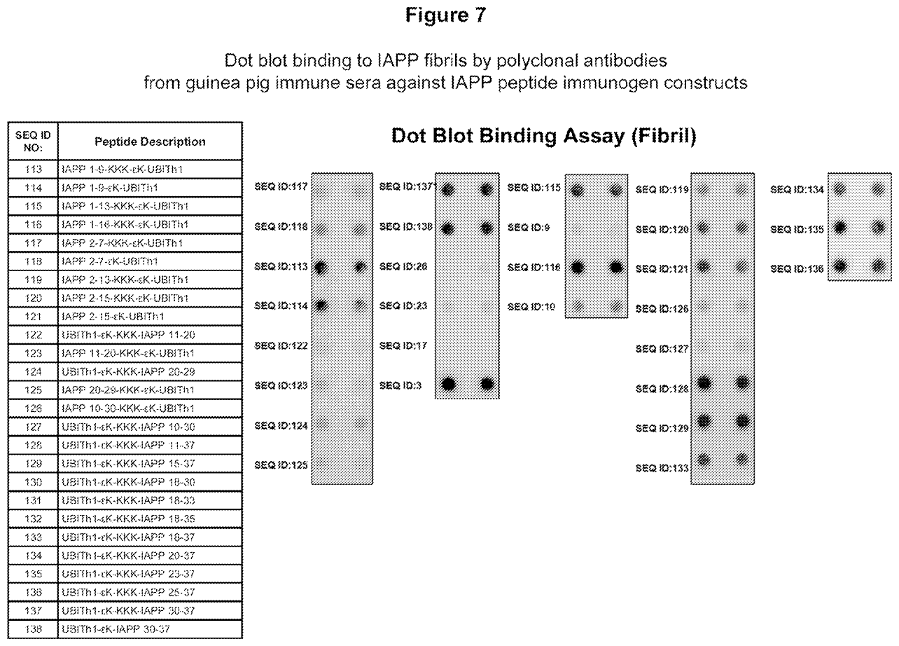

Antibodies elicited by the peptide immunogen constructs of SEQ ID NOs: 113-138 were evaluated for their abilities to bind to IAPP fibril. FIG. 7 depicts the IAPP fibril binding profiles by dot blot binding assay with guinea pig immune sera collected at 12 wpi (all samples run in duplicate). The results demonstrate that the peptide immunogen constructs derived from, or containing, the C-terminal region of IAPP (e.g., SEQ ID NOs: 128, 129, and 134-138) had the strongest binding profile to the IAPP oligomer compared to the other peptide immunogen constructs.

viii) Inhibition of IAPP Aggregation by Anti-IAPP Antibodies from 12 Wpi Guinea Pig Immune Sera to Show Inhibition of Fibrillation from Monomer to Fibril and Oligomer to Fibril.

The disclosed peptide immunogen constructs and antibodies elicited therefrom were tested for their ability to inhibit IAPP aggregation from (a) monomer to fibril and (b) oligomer to fibril. Specifically, a Thioflavin T (ThT) fluorescence assay was performed to show inhibition of fibrillation from monomer to fibril or oligomer to fibril.

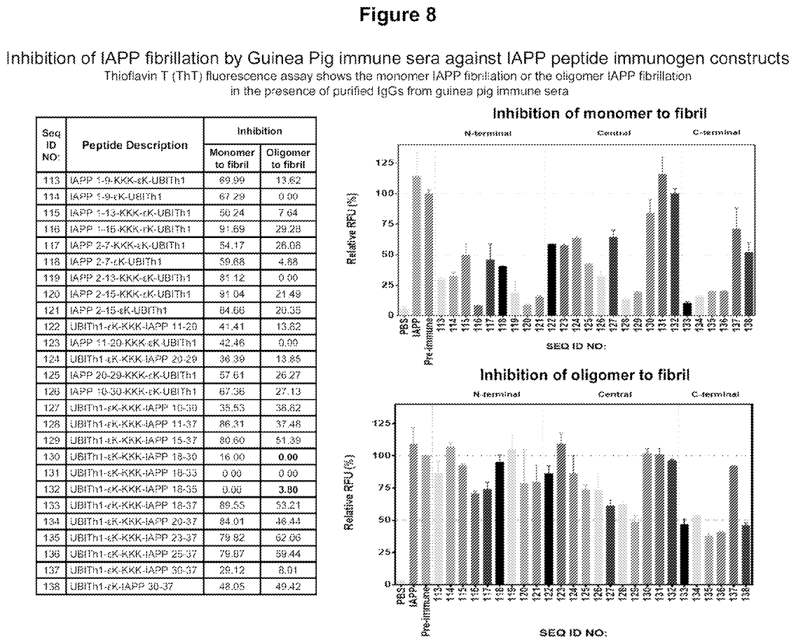

The dot blot binding profiles from 12 wpi immune sera against full-length IAPP monomer, oligomer, and fibril (based on FIGS. 5-7) were arranged according to their relative strengths, as summarized in Table 8 for ease of pattern recognition. Significant inhibition of fibrillation from monomer and from oligomers were found, as shown in FIG. 8. Preferential inhibition was found with constructs covering IAPP epitopes derived from, or containing, the central to C-terminal region of IAPP, which are aggregation prone. The N-terminal region, which is involved in membrane binding by IAPP, was found to inhibit fibril formation more weakly compared to the central and C-terminal regions.

FIG. 8 depicts inhibition of IAPP aggregation by anti-IAPP antibodies from 12 wpi guinea pig immune sera with corresponding IAPP peptide immunogen constructs of SEQ ID NOs: 113-138. All samples were run in duplicates.

### EXAMPLE 7

### Assessment of Functional Properties of Antibodies Elicted by the IAPP Peptide Immunnogen Constructs and Formulations Thereof in an Ex-Vivo Mode for Inhibition of Cell Toxicity Towards RIN-M5Fs Cells

After demonstration of the high immunogenicity and cross-reactivities of the antibodies purified from immune sera of guinea pigs immunized with IAPP immunogen constructs, as shown in Tables 4, 5, 6, 7, and 8, the following study was performed to assess the ability of antibodies produced by the disclosed peptide immunogen constructs to suppress cell cytotoxicity of IAPP oligomers in RIN-M5Fs cells.

Specifically, the viability of RIN-m5Fs cells was assessed after exposure to 40 μM aggregated IAPP oligomers in the presence of either pre-immune sera or anti-IAPP antibodies from 12 wpi guinea pig immune sera immunized with an IAPP peptide immunogen construct (SEQ ID NOs: 113-138). A control sample of RIN-m5Fs cells was exposed to PBS (where no IAPP oligomer was added to the cell culture), to determine maximum cell viability.

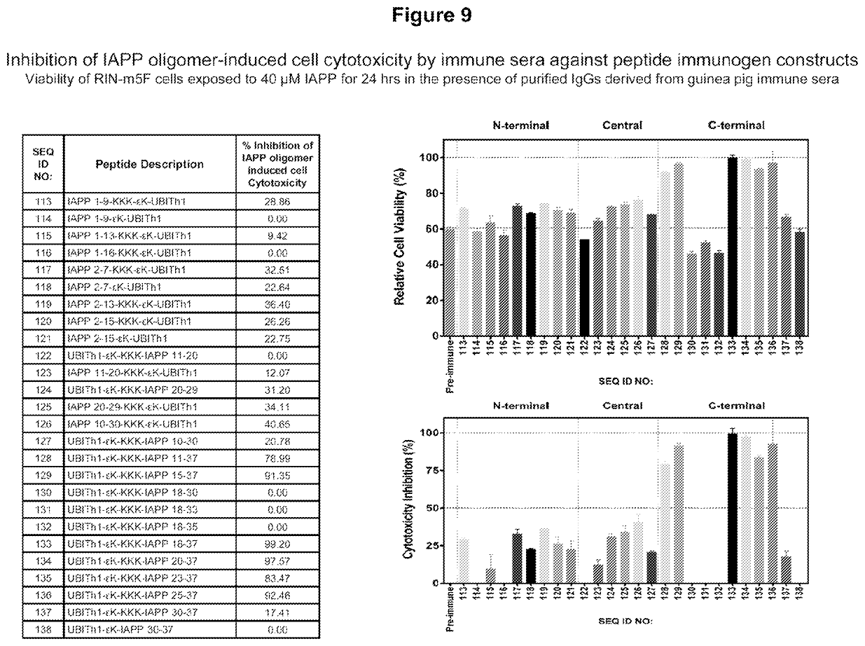

The top bar graph in FIG. 9 depicts the cell viability of RIN-m5Fs cells exposed to aggregated IAPP oligomers in the presence of either pre-immune sera or anti-IAPP antibodies elicited by IAPP peptide immunogen constructs (SEQ ID NOs: 113-138) relative to the PBS control (the dotted line at 100% in the top bar graph corresponds the cell viability of the PBS control). Purified antibody preparations from pooled pre-immune sera was used as a negative control, corresponding to maximal cell toxicity (approximately 60% cell viability). The relative cell viabilities for RIN-m5Fs cells exposed to aggregated IAPP oligomers in the presence of antibodies elicited by SEQ ID NOs: 113-138 are also shown in the top bar graph.

The cytotoxicity inhibition percentage (%) for each of the experimental samples was determined using the following equation:

$$\frac{\begin{matrix}( cell viability of target group - \\ cell viability of pre - immune group )\end{matrix}}{\begin{matrix}( cell viability of PBS group - \\ cell viability of pre - immune group )\end{matrix}} \times 100 = Cytotox . Inhib . \%$$

The cytotoxicity inhibition percentages of each of the samples is reported in the bottom bar graph and table shown in FIG. 9. The samples that had a negative cytotoxicity inhibition percentage, corresponding to cell viability values below the pre-immune sera (i.e., SEQ ID NOs: 114, 116, 122, 130-132, and 138), were assigned a value of 0.00% inhibition.

Significant protection/inhibition of cell cytotoxicity was observed with IAPP peptide immunogen constructs having B cell epitopes covering the C-terminal region, followed by those from the N-terminal membrane interaction region, then the central alphα-helical region. The peptide immunogen constructs from the C-terminal region (i.e., SEQ ID NOs: 128, 129, 133, 134, and 135) demonstrated the highest suppression/inhibition of cell cytotoxicity exerted by the IAPP oligomers. Cytotoxicity inhibition for each sample is also reported in Table 8 for ease of pattern recognition.

In summary, all immunological and functional features of the polyclonal antibodies from immune sera directed against IAPP peptide immunogen constructs, as summarized in Table 8, provide valuable references for testing of IAPP vaccine formulations to demonstrate efficacy in preventative and therapeutic modes through intervention by active immunization with IAPP peptide immunogen constructs.

### EXAMPLE 8

### Assessment of IAPP Peptide Immunogen Constructs in Both Preventive and Therapeutic Modes on a Type II Diabetes Mellitus (T2D) Model in FVB/N-Tg (Ins2-IAPP) RHFSoel/J Mice

Candidate IAPP peptide immunogen constructs and the formulations thereof along with a placebo group were validated in two transgenic mice models (hIAPP^{+/+}TG MICE and hIAPP^{+/-}TG MICE) expressing hIAPP: (1) FVB/hIAPP (homozygous) RHFSoel/J mice exposed to standard diet; and (2) FVB/hIAPP (hemizygous) RHFSoel/J mice exposed to high-fat and high-sucrose diet (HFFD).

Mice homozygous for the RIPHAT transgene are viable and fertile, with expression of hIAPP under the regulatory control of the rat insulin II promoter. While huIAPP RNA from the transgene is observed in pancreas, kidney, and stomach, h-IAPP protein is reported only in pancreas tissues. Homozygous males spontaneously develop diabetes mellitus due to beta-cell death, associated with impaired insulin secretion (hypoinsulinemia), hyperglycemia, and abnormal intracellular aggregates of h-IAPP (the donating investigator reports that extracellular aggregates are not found on this strain background). Homozygous male onset is between 4-8 weeks of age with death around 16 weeks of age. Homozygous females exhibit a less severe phenotype. The RIPHAT transgenic mice may be useful in studying the beta-cell destruction and islet amyloid deposition associated with non-insulin-dependent diabetes mellitus (NIDDM) or type II diabetes, as well as beta-cell apoptosis and characteristics of the endoplasmic reticulum (ER) stress pathway.

Mice hemizygous for the RIPHAT transgene, exposed to high-fat and high-sucrose diet (HFFD), have also been validated as a diabetes-associated phenotype transgenic mice model having over-expressed human IAPP with amyloid self-assembly ability.

Both Homozygote mice (hIAPP^{+/+}TG MICE) FVB/N-Tg (Ins2-IAPP) RHFSoel/J and hemizygote mice (hIAPP^{+/-}TG MICE): FVB/hIAPP (hemizygous) RHFSoel/J can be purchased from Jackson Laboratory. Due to the active immunization nature of the IAPP peptide immunogen constructs, and the rapid onset between 4-8 weeks of age with death around 16 weeks of age associated with the homozygote mice, the hemizygote mice, maintained by high-fat and high-sucrose (HFFD) diet, with a longer onset after 12 weeks, were selected for testing in this efficacy study. This study involved preventive and therapeutic disease interventions by assessing the beta-cell mass and hIAPP amyloid load in the pancreas as well as plasma levels of hIAPP, and functional tests of glucose metabolism and insulin secretion. More specifically, antibody titers, and other diabetes-associated parameters such as fasting blood glucose levels, serum concentrations of insulin and IAPP were monitored. In addition, hIAPP oligomers induced cytotoxicity towards β-cells were also assessed over the course of the study.

All experimental and handling procedures were performed under the supervision and approval of the UBI Asia Institutional Animal Care and Use Committee (IACUC), in accordance with NIH guidelines for the humane treatment of animals.

Blood glucose concentrations were examined after an 8 hour fast every 7 days. Values were measured from a tail-tip blood sample by a freestyle blood glucose meter (Accu-chek Performa). Mice were sacrificed under anesthesia and the pancreas was removed. Insulin was extracted from pancreas by incubating the pancreas, diced into small fragments and homogenized, in 5 mL acid alcohol (1.5% HCl in 70% EtOH) at 4° C. for several days and neutralized with 1 M Tris buffer in a 1:1 ratio. Since the extraction was performed in acid alcohol, the addition of protease inhibitors was not required. Insulin levels were determined using an ultrasensitive insulin ELISA kit (Mercodia). Blood samples were collected by submandibular bleeding. Antibodies serum titers were evaluated by ELISA.

### Tissue Collection and Analysis:

Sedated mice were perfused through the heart with 20 mL of 4% paraformaldehyde, pancreas was removed in cold PBS, weighed and fixed in 4% paraformaldehyde, 4° C. for 24 hours and embedded in paraffin. Histological sections (4 μm) were deparaffinized then washed with Tris-buffered saline (TBS)/0.1% TWEEN® 20, then blocked with TBS/0.2% Triton X-100/3% BSA/2% normal donkey serum (Jackson Immunoresearch Laboratories, West Grove, PA) for 3 hours at room temperature. Sections were treated with human IAPP antibody (E-5, Santa Cruz Biotechnology) or Insulin antibody (H-86, Santa Cruz biotechnology). Images were acquired using LSM 510 Meta confocal laser scanning microscope (Carl Zeiss Jena, Germany).

### Antibodies Purification

Antibodies from treated and mock groups were purified by protein-A/G column (GE healthcare). Serum was diluted 1:20 with loading buffer (20 mM Na₂HPO₄, 2 mM NaH₂PO₄ pH 7) and loaded onto a 5-ml protein-A/G column, flow throw was collected and reloaded 3 times. Bound antibody was eluted with 0.1 M of citric acid (pH 3.0) and neutralized with 1 M Tris-HCl (pH 9.0) for 1 ml of eluate, 200 μl of Tris buffer was added. Protein-containing fractions were combined, dialyzed against 2 liters PBS buffer (16 hours, 4° C.). Antibodies concentration was determined using Bradford reagent (Sigma-Aldrich).

### Antibodies Neutralizing Effect Against Rin-M Cell Toxicity Exerted by IAPP Oligomers.

Rin-m cells (2×10⁵ cells/ml) were cultured in 96-well microplates (100 μL/well) and incubated overnight at 37° C. Human oligomers (5 μM, total peptide) were added to each well in the presence of the antibodies at various concentrations. Each measurement was repeated four times. A control measurement with antibodies alone at the highest concentration was performed to refute any effect of antibodies on cell viability. Following incubation for 6 hours at 37° C., cell viability was evaluated using MTT assay.

### Statistical Analysis

Quantitative results are shown as means±SD. The statistical analysis was performed by Student's t-test between control and tested groups. P value of ≤0.05 was considered significant. *Pv≤0.05, **Pv≤0.005 and ***Pv≤0.0

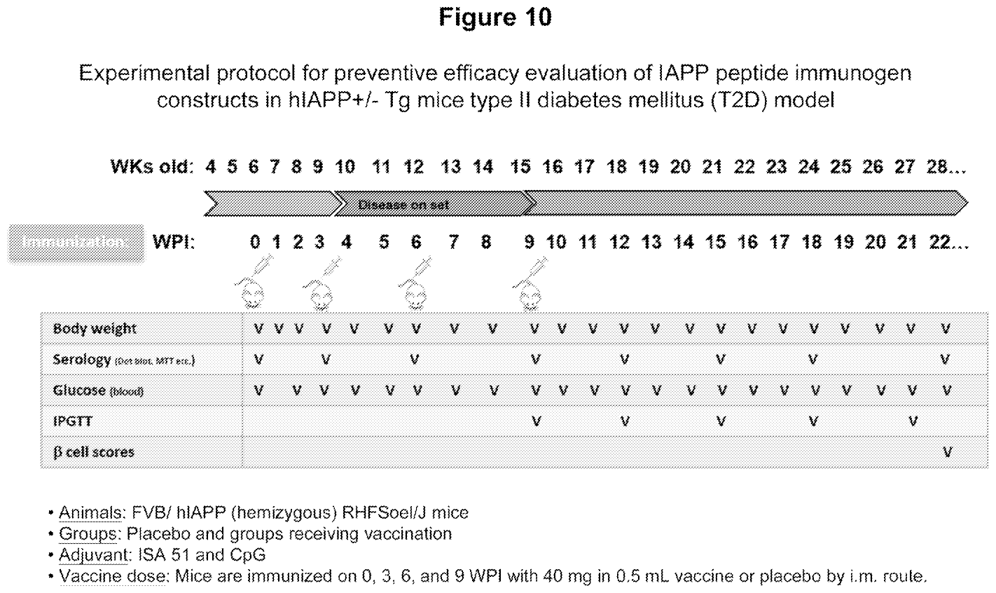

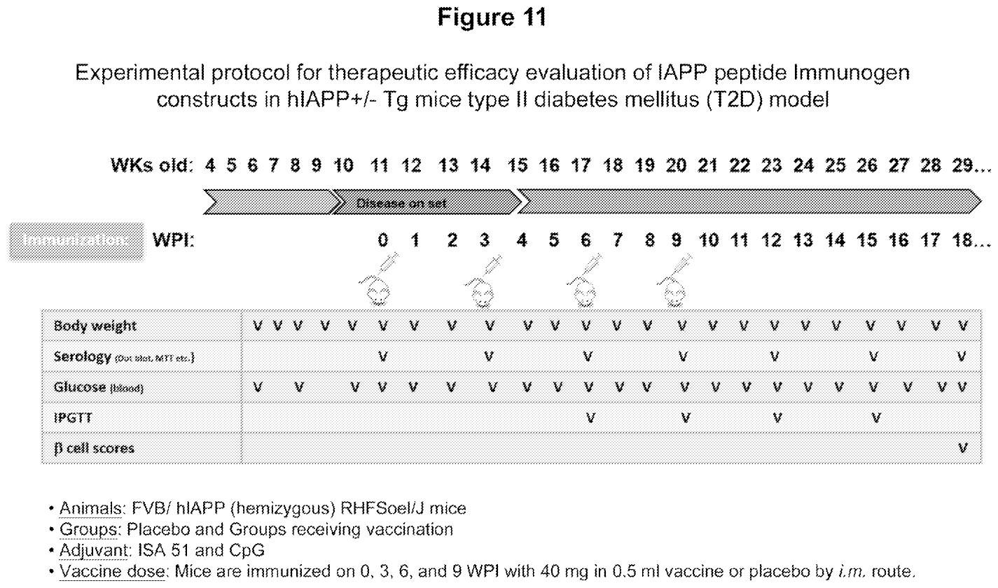

Experimental protocols for preventative and therapeutic efficacy evaluation of IAPP peptide immunogen constructs in hIAPP+/−Tg mice type II diabetes mellitus (T2D) model are shown in FIGS. 10 and 11 respectively.

A total of 10 mice per group will be used for the study with one being the placebo group. Mice in the experimental groups will be injected with the corresponding IAPP peptide immunogen constructs formulated with ISA 51 and CpG at 40 μg/0.5 mL dose for prime and boost immunizations under intramuscular route. A total of four doses will be administered on 0, 3, 6 and 9 WPI. All mice would have free access to high-fat and high-sucrose diet (HFFD) and water. The mice are to be bled on 0, 3, 6, 9, 12, 15, 18 and 22 WPI to test for efficacy parameters including antibody titers by ELISA and dot plot assays and MTT cell viability assay. Clinical symptoms of T2D, including weight gain and hyperglycemia are assessed weekly. Blood glucose concentrations are examined after 8-10 hour fasting. For intraperitoneal glucose tolerance test (IPGTT) on 9, 12, 15, 18 and 21 WPI, animals are to receive glucose 1 mg/g BW through intraperitoneal injection after 8-10 hours fasting and collected about 30 μL of serum from tail vein at 0, 15, 30, 60, 90 and 120 minutes. The insulin content and hIAPP accumulation in the pancreas are administrated at the end of the study.

The insulin content and hIAPP accumulation in the pancreas are suggested to have an important role in the T2D pathogenesis. Immunohistochemical staining method was applied to assess the pancreases tissue. At the end of the study, mice were sacrificed under anesthesia and the pancreas was removed in cold PBS, weighed and fixed in 4% paraformaldehyde, 4° C. 24 hours and embedded in paraffin. Histological sections (4 μm) were deparaffinized then processed for microwave-enhanced antigen retrieval. Slide-mounted sections immersed in Antigen Retrieval Citrate Solution (Scytek) were heated until boiling in a microwave oven at maximum power and cooled down to room temperature for 30 min. Endogenous peroxidase activity was blocked with 3% hydrogen peroxide/PBS for 10 min. and then washed with Tris-buffered saline (TBS)/0.1% Tween-20, then blocked with TBS/0.2% Triton X-100/3% BSA/2% normal donkey serum (Jackson Immunoresearch Laboratories, West Grove, PA) for 3 hours at room temperature. Sections were treated with human LAPP antibody (E-5, Santa Cruz Biotechnology) or Insulin antibody (H-86, Santa Cruz biotechnology). Images were acquired using LSM 510 Meta confocal laser scanning microscope (Carl Zeiss Jena, Germany).

TABLE 1
Amino Acid Sequences of IAPP and Fragments Thereof Employed in Serological Assays

| | SEQ | |
|---|---|---|
| Amino Acid positions | ID NO: | Sequence |
| Human PreProIAPP | 1 | MGILK LQVFL IVLSV ALNHL KATPI ESHQV EKRKC NTATC |
| | | ATQRL ANFLV HSCNN FGAIL SSTNV GSNTY GKRNA VEVLK |
| | | REPLN YLPL |
| Human ProIAPP | 2 | TPIES HQVEK RKCNT ATCAT QRLAN FLVHS CNNFG AILSS |
| | | TNVGS NTYGK RNAVE VLKRE PLNYL PL |
| Human IAPP (Amylin) | 3 | KCNTA TCATQ RLANF LVHSS NNFGA ILSST NVGSN TY |
| Cat IAPP | 4 | KCNTA TCATQ RLANF LIRSS NNLGA ILSPT NVGSN TY |
| Macaque IAPP | 5 | KCNTA TCATQ RLANF LVRSS NNFGT ILSST NVGSD TY |
| Rat/Mouse IAPP | 6 | KCNTA TCATQ RLANF LVRSS NNLGP VLPPT NVGSN TY |
| Guinea pig IAPP | 7 | KCNTA TCATQ RLTNF LVRSS HNLGA ALLPT DVGSN TY |
| IAPP 1-9 | 8 | KCNTA TCAT |
| IAPP ₁₋₁₃ | 9 | KCNTA TCATQ RLA |
| IAPP ₁₋₁₆ | 10 | KCNTA TCATQ RLANF L |
| IAPP ₂₋₇ | 11 | CNTAT |
| IAPP ₂₋₁₃ | 12 | CNTAT CATQR LA |
| IAPP ₂₋₁₅ | 13 | CNTAT CATQR LANF |
| IAPP ₁₁₋₂₀ | 14 | RLANF LVHSS |
| IAPP ₂₀₋₂₉ | 15 | SNNFG AILSS |
| IAPP ₁₀₋₃₀ | 16 | QRLAN FLVHS SNNFG AILSS T |
| IAPP ₁₁₋₃₇ | 17 | RLANF LVHSS NNFGA ILSST NVGSN TY |
| IAPP ₁₅₋₃₇ | 18 | FLVHS SNNFG AILSS TNVGS NTY |
| IAPP ₁₈₋₃₀ | 19 | HSSNN FGAIL SST |
| IAPP ₁₈₋₃₃ | 20 | HSSNN FGAIL SSTNV G |
| IAPP ₁₈₋₃₅ | 21 | HSSNN FGAIL SSTNV GSN |
| IAPP ₁₈₋₃₇ | 22 | HSSNN FGAIL SSTNV GSNTY |
| IAPP ₂₀₋₃₇ | 23 | SNNFG AILSS TNVGS NTY |
| IAPP ₂₃₋₃₇ | 24 | FGAIL SSTNV GSNTY |
| IAPP ₂₅₋₃₇ | 25 | AILSS TNVGS NTY |
| IAPP ₃₀₋₃₇ | 26 | TNVGS NTY |
| IAPP ₋₈₋₂ | 27 | ESHQV EKRKC |
| IAPP ₋₇₋₃ | 28 | SHQVE KRKCN |
| IAPP ₋₆₋₄ | 29 | HQVEK RKCNT |
| IAPP ₋₅₋₅ | 30 | QVEKR KCNTA |
| IAPP ₋₄₋₆ | 31 | VEKRK CNTAT |
| IAPP ₋₃₋₇ | 32 | EKRKC NTATC |
| IAPP ₋₂₋₈ | 33 | KRKCN TATCA |
| IAPP ₋₁₋₉ | 34 | RKCNT ATCAT |
| IAPP ₁₋₁₀ | 35 | KCNTA TCATQ |
| IAPP ₂₋₁₁ | 36 | CNTAT CATQR |
| IAPP ₃₋₁₂ | 37 | NTATC ATQRL |
| IAPP ₄₋₁₃ | 38 | TATCA TQRLA |
| IAPP ₅₋₁₄ | 39 | ATCAT QRLAN |
| IAPP ₆₋₁₅ | 40 | TCATQ RLANF |
| IAPP ₇₋₁₆ | 41 | CATQR LANFL |
| IAPP ₈₋₁₇ | 42 | ATQRL ANFLV |
| IAPP ₉₋₁₈ | 43 | TQRLA NFLVH |
| IAPP ₁₀₋₁₉ | 44 | QRLAN FLVHS |
| IAPP ₁₁₋₂₀ | 45 | RLANF LVHSS |
| IAPP ₁₂₋₂₁ | 46 | LANFL VHSSN |
| IAPP ₁₃₋₂₂ | 47 | ANFLV HSSNN |
| IAPP ₁₄₋₂₃ | 48 | NFLVH SSNNF |
| IAPP ₁₅₋₂₄ | 49 | FLVHS SNNEG |
| IAPP ₁₆₋₂₅ | 50 | LVHSS NNFGA |
| IAPP ₁₇₋₂₆ | 51 | VHSSN NFGAI |
| IAPP ₁₈₋₂₇ | 52 | HSSNN FGAIL |
| IAPP ₁₉₋₂₈ | 53 | SSNNF GAILS |
| IAPP ₂₀₋₂₉ | 54 | SNNFG AILSS |
| IAPP ₂₁₋₃₀ | 55 | NNFGA ILSST |
| IAPP ₂₂₋₃₁ | 56 | NFGAI LSSTN |
| IAPP ₂₃₋₃₂ | 57 | FGAIL SSTNV |
| IAPP ₂₄₋₃₃ | 58 | GAILS STNVG |
| IAPP ₂₅₋₃₄ | 59 | AILSS TNVGS |
| IAPP ₂₆₋₃₅ | 60 | ILSST NVGSN |
| IAPP ₂₇₋₃₆ | 61 | LSSTN VGSNT |
| IAPP ₂₈₋₃₇ | 62 | SSTNV GSNTY |
| IAPP ₂₉₋₃₈ | 63 | STNVG SNTYG |
| IAPP ₃₀₋₃₉ | 64 | TNVGS NTYGK |
| IAPP ₃₁₋₄₀ | 65 | NVGSN TYGKR |
| IAPP ₃₂₋₄₁ | 66 | VGSNT YGKRN |
| IAPP ₃₃₋₄₂ | 67 | GSNTY GKRNA |
| IAPP ₃₄₋₄₃ | 68 | SNTYG KRNAV |
| IAPP ₃₅₋₄₄ | 69 | NTYGK RNAVE |
| Flexible Hinge Spacer | 70 | PPXPXP |
| Spacer1 | 71 | ϵK-KKK |
| Spacer2 | 72 | KKK-ϵK |
| Human α-CGRP ₁₋₃₇ | 168 | ACDTA TCVTH RLAGL LSRSG GVVKN NFVPT NVGSK AF |
| Human Abeta ₁₋₄₂ | 169 | DAEFR HDSGY EVHHQ KLVFF AEDVG SNKGA IIGLM VGGVV IA |

TABLE 2
Amino Acid Sequences of Pathogen Protein Derived Th Epitopes Including Idealized
Artificial Th Epitopes for Employment in the Design of IAPP Peptide Immunogen
Constructs

| | SEQ ID | |
|---|---|---|
| Description | NO: | Sequence |
| Clostridium tetani1 Th (UBITh ®6) | 73 | KKQYIKANSKFIGITEL |
| MvF1 Th (UBITh@5) | 74 | LSEIKGVIVHRLEGV |
| Bordetella pertussis Th (UBITh ®7) | 75 | GAYARCPNGTRALTVAELRGNAEL |
| Clostridium tetani2 Th | 76 | WVRDIIDDETNESSQKT |
| Diphtheria Th | 77 | DSETADNLEKTVAALSILPGHGC |
| Plasmodium falciparum Th | 78 | DHEKKHAKMEKASSVENVVNS |
| Schistosoma mansoni Th | 79 | KWFKTNAPNGVDEKHRH |
| Cholera Toxin Th | 80 | ALNIWDRFDVFCTLGATTGYLKGNS |
| MvF2 Th | 81 | ISEIKGVIVHKIEGI |
| KKKMvF3 Th | 82 | KKKISISEIKGVIVHKIEGILF |
| | 83 | T RT TR T |
| | 84 | KKKISIXEIXXVIVXXIEXILF |
| HBsAg1 Th | 85 | KKKLFLLTKLLTLPQSLD |
| | 86 | RRRIKII RII I L IR |
| | 87 | VRVV VV V I V |
| | 88 | F FF FF F V F |
| | 89 | F |
| | 90 | XXXXXXXTXXXTXPXSXX |
| MvF4 Th (UBITh@3) | 91 | ISISEIKGVIVHKIETILF |
| | 92 | T RT TR |
| | 93 | ISIXEIXXVIVXXIETILF |
| HBsAg2 Th | 94 | KKKIITITRIITIPQSLD |
| | 95 | FFLL L ITTI |
| | 96 | KKKXXXXTRIXTIXXXXD |
| MvF5 Th (UBITh ®1) | 97 | ISITEIKGVIVHRIETILF |
| HBsAg3 Th (UBITh ®2) | 98 | KKKIITITRIITIITTID |
| Influenza MP1_1 Th | 99 | FVFTLTVPSER |
| Influenza MP1_2 Th | 100 | SGPLKAEIAQRLEDV |
| Influenza NSP1 Th | 101 | DRLRRDQKS |
| EBV BHRF1 Th | 102 | AGLILSLLVICSYLFISRG |
| Clostridium tetani TT1 Th | 103 | QYIKANSKFIGITEL |
| EBV EBNA-1 Th | 104 | PGPLRESIVCYFMVELQTHI |
| Clostridium tetani TT2 Th | 105 | FNNFTVSFWLRVPKVSASHLE |
| Clostridium tetani TT3 Th | 106 | KFIIKRYTPNNEIDSE |
| Clostridium tetani TT4 Th | 107 | VSIDKFRIFCKALNPK |
| EBV CP Th | 108 | VPGLYSPCRAFFNKEELL |
| HCMV IE1 Th | 109 | DKREMWMACIKELH |
| EBV GP340 Th | 110 | TGHGARTSTEPTTDY |
| EBV BPLF1 Th | 111 | KELKRQYEKKLRQ |
| EBV EBNA-2 Th | 112 | TVFYNIPPMPL |
| MvF Th | 171 | DLSDLKGLLLHKLDGL |
| (SSAL1 Th1) | 172 | EI EIR III RIE I |
| | 173 | V V VVV V V |
| | 174 | F F FFF F F |
| | 175 | XXSXXXGXXXHXXXGX |
| MvF3 Th | 176 | ISISEIKGVIVHKIEGILF |
| | 177 | T RT TR T |
| | 178 | ISIXEIXXVIVXXIEXILF |
| KKKMvF5 Th (UBITh ®1a) | 179 | KKKISITEIKGVIVHRIETILF |
| HBsAg Th (UBITh ®4) | 180 | FELLTRILTIPQSLD |
| KKK-HBsAg Th | 181 | KKKFELLTRILTIPQSLD |
| HBsAg Th | 182 | FELLTRILTIPQSL |

TABLE 3
Amino Acid Sequences of IAPP Peptide Immunogen Constructs

| | SEQ ID | |
|---|---|---|
| Peptide Description | NO | Sequence |
| IAPP₁₋₉-KKK-ϵK-UBITh1 | 113 | KCNTATCAT -KKK-ϵK-UBITh1 |
| IAPP₁₋₉-ϵK-UBITh1 | 114 | KCNTATCAT -ϵK-UBITh1 |
| IAPP₁₋₁₃-KKK-ϵK-UBITh1 | 115 | KCNTATCATQRLA -KKK-ϵK-UBITh1 |
| IAPP₁₋₁₆-KKK-ϵK-UBITh1 | 116 | KCNTATCATQRLANFL -KKK-ϵK-UBITh1 |
| IAPP₂₋₇-KKK-ϵK-UBITh1 | 117 | CNTATC -KKK-ϵK-UBITh1 |
| IAPP₂₋₇-ϵK-UBITh1 | 118 | CNTATC -ϵK-UBITh1 |
| IAPP₂₋₁₃-KKK-ϵK-UBITh1 | 119 | CNTATCATQRLA -KKK-ϵK-UBITh1 |
| IAPP₂₋₁₅-KKK-ϵK-UBITh1 | 120 | CNTATCATQRLANF -KKK-ϵK-UBITh1 |
| IAPP₂₋₁₅-ϵK-UBITh1 | 121 | CNTATCATQRLANF -ϵK-UBITh1 |
| UBITh1-ϵK-KKK-IAPP₁₁₋₂₀ | 122 | UBITh1-ϵK-KKK- RLANFIVHSS |
| IAPP₁₁₋₂₀-KKK-ϵK-UBITh1 | 123 | RLANFLVHSS -KKK-ϵK-UBITh1 |
| UBITh1-ϵK-KKK-IAPP₂₀₋₂₉ | 124 | UBITh1-ϵK-KKK- SNNFGAILSS |
| IAPP₂₀₋₂₉-KKK-ϵK-UBITh1 | 125 | SNNFGAILSS -KKK-ϵK-UBITh1 |
| IAPP₁₀₋₃₀-KKK-ϵK-UBITh1 | 126 | QRLANFLVHSSNNFGAILSST -KKK-ϵK-UBITh1 |
| UBITh1-ϵK-KKK-IAPP₁₀₋₃₀ | 127 | UBITh1-ϵK-KKK- QRLANFLVASSNNFGAILSST |
| UBITh1-ϵK-KKK-IAPP₁₁₋₃₇ | 128 | UBITh1-ϵK-KKK- RLANFLVHSSNNFGAILSSTNVGSNTY |
| UBITh1-ϵK-KKK-IAPP₁₅₋₃₇ | 129 | UBITh1-ϵK-KKK- FLVHSSNNFGAILSSTNVGSNTY |
| UBITh1-K-KKK-IAPP₁₈₋₃₀ | 130 | UBITh1-ϵK-KKK- HSSNNFGAILSST |
| UBITh1-ϵK-KKK-IAPP₁₈₋₃₃ | 131 | UBITh1-ϵK-KKK- HSSNNFGAILSSTNVG |
| UBITh1-ϵK-KKK-IAPP₁₈₋₃₅ | 132 | UBITh1-ϵK-KKK- HSSNNFGAILSSTNVGSN |
| UBITh1-ϵK-KKK-IAPP₁₈₋₃₇ | 133 | UBITh1-ϵK-KKK- HSSNNFGAILSSTNVGSNTY |
| UBITh1-ϵK-KKK-IAPP₂₀₋₃₇ | 134 | UBIThl-ϵK-KKK- SNNFGAILSSTNVGSNTY |
| UBITh1-ϵK-KKK-IAPP₂₃₋₃₇ | 135 | UBITh1-ϵK-KKK- FGAILSSTNVGSNTY |
| UBITh1-ϵK-KKK-IAPP₂₅₋₃₇ | 136 | UBIThl-ϵK-KKK- AILSSTNVGSNTY |
| UBITh1-ϵK-KKK-IAPP₃₀₋₃₇ | 137 | UBIThl-ϵK-KKK- TNVGSNTY |
| UBITh1-ϵK-IAPP₃₀₋₃₇ | 138 | UBITh1-ϵK- TNVGSNTY |
| UBITh1-ϵK-KKK-Guinea | 139 | UBIThl-ϵK-KKK- RSSHNLGAALLPTDVGSNTY |
| Pig IAPP₁₈₋₃₇ | | |
| HBsAg3 Th (UBITh2)- | 140 | KKKIITITRIITIITTID-ϵK-KKK- HSSNNFGAILSSTNVGSNTY |
| ϵK-KKK-IAPP₁₈₋₃₇ | | |
| MvF4 Th (UBITh3)-ϵK- | 141 | ISISEIKGVIVHKIETILF-ϵK-KKK- HSSNNFGAILSSTNVGSNTY |
| KKK-IAPP₁₈₋₃₇ | | T RT TR |
| Clostridium tetani1 | 142 | KKQYIKANSKFIGITEL-ϵK-KKK- HSSNNFGAILSSTNVGSNTY |
| Th-ϵK-KKK-IAPP₁₈₋₃₇ | | |
| MvF 1Th-ϵK-KKK- | 143 | LSEIKGVIVHRLEGV-ϵK-KKK- HSSNNFGAILSSTNVGSNTY |
| IAPP₁₈₋₃₇ | | |
| Bordetella pertussis | 144 | GAYARCPNGTRALTVAELRGNAEL-ϵK-KKK- HSSNNFGAILSSTNVGSNTY |
| Th-ϵK-KKK-IAPP₁₈₋₃₇ | | |
| Clostridium tetani2 | 145 | WVRDIIDDFTNESSQKT-ϵK-KKK- HSSNNFGAILSSTNVGSNTY |
| Th-ϵK-KKK-IAPP₁₈₋₃₇ | | |
| Diphtheria Th-ϵK- | 146 | DSETADNLEKTVAALSILPGHGC-ϵK-KKK- HSSNNFGAILSSTNVGSNTY |
| KKK-IAPP₁₈₋₃₇ | | |
| Plasmodium falciparum | 147 | DHEKKHAKMEKASSVFNVVNS-ϵK-KKK- HSSNNFGAILSSTNVGSNTY |
| Th-ϵK-KKK-IAPP₁₈₋₃₇ | | |
| Schistosoma mansoni | 148 | KWFKTNAPNGVDEKARH-ϵK-KKK- HSSNNFGAILSSTNVGSNTY |
| Th-ϵK-KKK-IAPP₁₈₋₃₇ | | |
| Cholera Toxin Th- | 149 | ALNIWDRFDVFCTLGATTGYLKGNS-ϵK-EKK-HSSNNFGAILSSTNVGSNTY |
| ϵK-KKK-IAPP₁₈₋₃₇ | | |
| MvF2 Th-ϵK-KKK- | 150 | ISEIKGVIVHKIEGI-ϵK-KKK- HSSNNFGAILSSTNVGSNTY |
| IAPP₁₈₋₃₇ | | |
| KKKMvF3 Th-ϵK- | 151 | KKKISISEIKGVIVHKIEGILF-ϵK-KEK- HSSNNFGAILSSTNVGSNTY |
| KKK-IAPP₁₈₋₃₇ | | T RT TR T |
| HBsAg1 Th-ϵK- | 152 | KKKLFLLTKLLTLPQSLD-ϵK-KKK- |
| KKK-IAPP₁₈₋₃₇ | | RRRIKII RII I L IR |
| | | VRVV VV V I V |
| | | F FF FF F V F |
| | | F |
| HBsAg2 Th-ϵK- | 153 | KKKIITITRIITIPQSLD-ϵK-KKK- HSSNNFGAILSSTNVGSNTY |
| KKK-IAPP₁₈₋₃₇ | | FFLL L ITTI |
| Influenza MP1_1 | 154 | FVFTLTVPSER-ϵK-KKK- HSSNNFGAILSSTNVGSNTY |
| Th-ϵK-KKK-IAPP₁₈₋₃₇ | | |
| Influenza MP1_2 | 155 | SGPLKAEIAQRLEDV-ϵK-KKK- HSSNNFGAILSSTNVGSNTY |
| Th-ϵK-KKK-IAPP₁₈₋₃₇ | | |
| Influenza NSP1 Th- | 156 | DRLRRDQKS-ϵK-KKK- HSSNNFGAILSSTNVGSNTY |
| ϵK-KKK-IAPP₁₈₋₃₇ | | |
| EBV BHRF1 Th- | 157 | AGLTLSLLVICSYLFISRG-ϵK-KKK- HSSNNFGAILSSTNVGSNTY |
| ϵK-KKK-IAPP₁₈₋₃₇ | | |
| Clostridium tetani | 158 | QYIKANSKFIGITEL-ϵK-KKK- HSSNNFGAILSSTNVGSNTY |
| TT1 Th-ϵK-KKK- | | |
| α-CGRP₁₈₋₃₇ | | |
| EBV EBNA-1 Th-ϵK- | 159 | PGPLRESIVCYFMVFLQTHI-ϵK-KKK- HSSNNFGAILSSTNVGSNTY |
| KKK-IAPP₁₈₋₃₇ | | |
| Clostridium tetani | 160 | FNNFTVSFWLRVPKVSASHLE-ϵK-KKK- HSSNNFGAILSSTNVGSNTY |
| TT2 Th-ϵK-KKK- | | |
| α-CGRP₁₈₋₃₇ | | |
| Clostridium tetani | 161 | KFIIKRYTPNNEIDSF-ϵK-KKK- HSSNNFGAILSSTNVGSNTY |
| TT3 Th-ϵK- | | |
| KKK-IAPP₁₈₋₃₇ | | |
| Clostridium | 162 | VSIDKFRIFCKALNPK-ϵK-KKK- HSSNNFGAILSSTNVGSNTY |
| tetani TT4 | | |
| Th-ϵK-KKK-IAPP₁₈₋₃₇ | | |
| EBV CP Th-ϵK- | 163 | VPGLYSPCRAFFNKEELL-ϵK-KKK- HSSNNFGAILSSTNVGSNTY |
| KKK-IAPP₁₈₋₃₇ | | |
| HCMV IE1 Th-ϵK- | 164 | DKREMWMACIKELH-ϵK-KKK- HSSNNFGAILSSTNVGSNTY |
| KKK-IAPP₁₈₋₃₇ | | |
| EBV GP340 Th-ϵK- | 165 | TGHGARTSTEPTTDY-ϵK-KKE- HSSNNFGAILSSTNVGSNTY |
| KKK-IAPP₁₈₋₃₇ | | |
| EBV BPLF1 Th-ϵK- | 166 | KELKRQYEKKLRQ-ϵK-KKK- HSSNNFGAILSSTNVGSNTY |
| KKK-IAPP₁₈₋₃₇ | | |
| EBV EBNA-2 Th-ϵK- | 167 | TVFYNIPPMPL-ϵK-KKK- HSSNNFGAILSSTNVGSNTY |
| KKK-IAPP₁₈₋₃₇ | | |

¹ Peptides are cyclized by cysteine disulfide bonds with the cysteines underlined.
² UBITH1 has the sequence of SEQ ID NO: 97

TABLE 4
Immunogenicity Assessment in Guinea Pigs of IAPP
Peptide Immunogen Constructs

| | SEQ | | IAPP (1-37) (SEQ ID NO: 3) | | | | | |
|---|---|---|---|---|---|---|---|---|
| | ID | Animal | ELISA Log10 Titer | | | | | |
| Immunogen | NO: | No | 0 w | 3 w | 6 w | 9 w | 12 w | 15 w |
| IAPP ₁₋₉-KKK-εK-UBITh1 | 113 | 7090 | 0.085 | 3.747 | 4.712 | 4.590 | 4.634 | 4.631 |
| | | 7091 | 0.086 | 3.695 | 4.948 | 4.964 | 5.121 | 4.993 |
| | | 7092 | 0.093 | 3.103 | 4.757 | 4.757 | 4.741 | 4.513 |
| IAPP ₁₋₉-εK-UBITh1 | 114 | 7093 | 0.090 | 3.405 | 5.151 | 4.605 | 4.838 | 4.816 |
| | | 7094 | 0.084 | 3.029 | 4.828 | 4.327 | 4.650 | 4.505 |
| | | 7095 | 0.081 | 3.647 | 4.968 | 4.694 | 4.716 | 4.786 |
| IAPP ₁₋₁₃-KKK-εK-UBITh1 | 115 | 7426 | 0.096 | 3.433 | 5.426 | 5.369 | 5.260 | 5.094 |
| | | 7427 | 0.082 | 3.278 | 5.492 | 5.058 | 4.905 | 4.956 |
| | | 7428 | 0.092 | 4.947 | 5.974 | 4.982 | 4.923 | 4.841 |
| IAPP ₁₋₁₆-KKK-εK-UBITh1 | 116 | 7432 | 0.104 | 3.664 | 5.304 | 4.898 | 5.041 | 4.936 |
| | | 7433 | 0.068 | 3.392 | 5.040 | 4.676 | 4.724 | 4.733 |
| | | 7434 | 0.070 | 4.473 | 5.630 | 5.018 | 4.964 | 4.892 |
| IAPP ₂₋₇-KKK-εK-UBITh1 | 117 | 7084 | 0.101 | 2.071 | 2.513 | 2.256 | 2.939 | 3.189 |
| | | 7085 | 0.117 | 0.000 | 2.745 | 2.226 | 2.701 | 3.138 |
| | | 7086 | 0.084 | 0.148 | 2.783 | 2.831 | 3.035 | 2.935 |
| IAPP ₂₋₇-εK-UBITh1 | 118 | 7087 | 0.094 | 2.816 | 3.434 | 3.765 | 3.990 | 3.669 |
| | | 7088 | 0.085 | 1.988 | 1.879 | 2.946 | 3.453 | 3.265 |
| | | 7089 | 0.079 | 2.795 | 4.518 | 3.451 | 4.144 | 3.760 |
| IAPP ₂₋₁₃-KKK-εK-UBITh1 | 119 | 7487 | 0.075 | 3.685 | 4.418 | 4.011 | 4.687 | 4.817 |
| | | 7488 | 0.075 | 3.252 | 4.180 | 4.140 | 3.829 | 4.542 |
| | | 7489 | 0.076 | 2.521 | 4.028 | 4.054 | 3.911 | 4.131 |
| IAPP ₂₋₁₅-KKK-εK-UBITh1 | 120 | 7490 | 0.051 | 2.458 | 3.553 | 3.648 | 4.137 | 4.465 |
| | | 7491 | 0.068 | 3.373 | 4.936 | 4.716 | 4.428 | 4.796 |
| | | 7492 | 0.055 | 2.404 | 4.989 | 4.238 | 4.479 | 4.631 |
| IAPP ₂₋₁₅-εK-UBITh1 | 121 | 7493 | 0.059 | 4.067 | 5.072 | 4.778 | 4.8854 | 4.904 |
| | | 7494 | 0.065 | 3.029 | 5.167 | 5.150 | 5.016 | 5.086 |
| | | 7495 | 0.067 | 2.876 | 6.971 | 3.654 | 3.521 | 3.821 |
| UBITh1-εK-KKK-IAPP ₁₁₋₂₀ | 122 | 7096 | 0.078 | 2.947 | 2.673 | 3.538 | 3.542 | 2.881 |
| | | 7097 | 0.080 | 1.156 | 2.438 | 1.483 | 2.051 | 1.471 |
| | | 7098 | 0.078 | 2.882 | 3.526 | 3.140 | 3.477 | 3.193 |
| IAPP ₁₁₋₂₀-KKK-εK-UBITh1 | 123 | 7099 | 0.084 | 1.921 | 4.004 | 2.674 | 3.729 | 2.918 |
| | | 7100 | 0.089 | 1.299 | 3.298 | 3.115 | 3.724 | 3.258 |
| | | 7101 | 0.084 | 2.267 | 3.242 | 3.417 | 4.394 | 4.059 |
| UBITh1-εK-KKK-IAPP ₂₀₋₂₉ | 124 | 7102 | 0.084 | 4.048 | 5.158 | 4.705 | 4.910 | 4.834 |
| | | 7103 | 0.086 | 4.633 | 4.898 | 4.739 | 4.998 | 4.877 |
| | | 7104 | 0.077 | 4.083 | 5.058 | 5.126 | 5.178 | 5.119 |
| IAPP ₂₀₋₂₉-KKK-εK-UBITh1 | 125 | 7105 | 0.077 | 4.219 | 5.199 | 4.766 | 4.834 | 4.746 |
| | | 7106 | 0.079 | 4.976 | 5.806 | 4.965 | 5.322 | 5.334 |
| | | 7107 | 0.087 | 3.207 | 4.906 | 4.851 | 4.774 | 4.534 |
| IAPP ₁₀₋₃₀-KKK-εK-UBITh1 | 126 | 7496 | 0.075 | 0.704 | 4.133 | 4.375 | 4.002 | 4.330 |
| | | 7497 | 0.081 | 2.553 | 3.577 | 3.481 | 3.394 | 3.433 |
| | | 7498 | 0.051 | 3.421 | 5.162 | 4.962 | 4.966 | 4.985 |
| UBITh1-εK-KKK-IAPP ₁₁₋₃₇ | 128 | 7502 | 0.064 | 2.961 | 6.335 | 7.125 | 7.498 | 7.189 |
| | | 7503 | 0.070 | 4.843 | 7.437 | 6.133 | 6.907 | 7.086 |
| | | 7504 | 0.073 | 5.376 | 9.530 | 7.256 | 9.549 | >10 |
| UBITh1-εK-KKK-IAPP ₁₅₋₃₇ | 129 | 7505 | 0.078 | 4.997 | >10 | >10 | >10 | >10 |
| | | 7506 | 0.055 | 5.287 | >10 | >10 | >10 | >10 |
| | | 7507 | 0.056 | 5.256 | >10 | >10 | >10 | >10 |
| UBITh1-εK-KKK-IAPP ₁₈₋₃₀ | 130 | 7793 | 0.082 | 4.767 | 5.121 | 5.263 | 5.920 | 5.498 |
| | | 7794 | 0.086 | 4.641 | 4.858 | 4.846 | 4.805 | 4.709 |
| | | 7795 | 0.086 | 4.422 | 4.944 | 4.931 | 4.931 | 4.792 |
| UBITh1-εK-KKK-IAPP ₁₈₋₃₃ | 131 | 7790 | 0.083 | 4.976 | 5.601 | 6.296 | 5.482 | 5.260 |
| | | 7791 | 0.073 | 4.722 | 5.108 | 5.054 | 4.902 | 4.834 |
| | | 7792 | 0.069 | 5.230 | 6.474 | 5.649 | 5.355 | 5.037 |
| UBITh1-εK-KKK-IAPP ₁₈₋₃₅ | 132 | 7787 | 0.099 | 4.898 | 5.519 | 5.411 | 5.393 | 5.121 |
| | | 7788 | 0.087 | 5.012 | 5.089 | 4.946 | 4.877 | 4.748 |
| | | 7789 | 0.082 | 5.016 | 5.207 | 5.070 | 5.164 | 5.100 |
| UBITh1-εK-KKK-IAPP ₁₈₋₃₇ | 133 | 7508 | 0.052 | 4.667 | 5.890 | 6.191 | 5.727 | 5.883 |
| | | 7509 | 0.051 | 4.566 | 8.492 | 7.262 | 6.858 | 7.336 |
| | | 7510 | 0.061 | 5.540 | >10 | 5.550 | 5.464 | 5.603 |
| UBITh1-εK-KKK-IAPP ₂₀₋₃₇ | 134 | 7511 | 0.073 | 5.258 | 9.125 | 5.524 | 5.639 | 5.657 |
| | | 7512 | 0.072 | 4.651 | 5.912 | 5.414 | 5.414 | 5.868 |
| | | 7513 | 0.061 | 4.722 | 5.668 | 5.186 | 5.190 | 5.237 |
| UBITh1-εK-KKK-IAPP ₂₃₋₃₇ | 135 | 7514 | 0.055 | 5.631 | >10 | 7.157 | 6.571 | 6.737 |
| | | 7515 | 0.055 | 3.854 | 6.173 | 5.467 | 5.692 | 7.210 |
| | | 7516 | 0.050 | 4.828 | >10 | 8.877 | 8.298 | >10 |
| UBITh1-εK-KKK-IAPP ₂₅₋₃₇ | 136 | 7517 | 0.051 | 4.779 | >10 | 9.740 | 8.732 | >10 |
| | | 7518 | 0.056 | 5.102 | >10 | 7.212 | 8.058 | 8.120 |
| | | 7519 | 0.070 | 4.883 | >10 | 8.684 | 7.053 | 7.296 |
| UBITh1-εK-KKK-IAPP ₃₀₋₃₇ | 137 | 7108 | 0.094 | 5.543 | >10 | 7.669 | 6.722 | 5.904 |
| | | 7109 | 0.086 | 4.794 | >10 | 8.483 | 8.111 | 6.898 |
| | | 7110 | 0.089 | 5.117 | >10 | 8.550 | 9.374 | 6.738 |
| UBITh1-εK-IAPP ₃₀₋₃₇ | 138 | 7111 | 0.084 | 4.646 | 5.535 | 5.248 | 5.135 | 5.009 |
| | | 7112 | 0.073 | 4.842 | 5.469 | 5.429 | 5.459 | 5.479 |
| | | 7113 | 0.075 | 5.123 | 6.443 | 5.577 | 5.616 | 5.195 |

TABLE 5
Lack of immunogenicity of selected IAPP B epitope sequences

| | | | IAPP (1-16) (SEQ ID NO: 10) | | | | | | IAPP (1-37) (SEQ ID NO: 3) | | | | | |
|---|---|---|---|---|---|---|---|---|---|---|---|---|---|---|
| | SEQ ID | Animal | ELISA Log₁₀ Titer | | | | | | ELISA Log10 Titer | | | | | |
| Immunogen | NO: | No | 0 w | 3 w | 6 w | 9 w | 12 w | 15 w | 0 w | 3 w | 6 w | 9 w | 12 w | 15 w |
| IAPP ₁₋₁₃ | 9 | 7429 | 0.095 | 0.000 | 0.613 | 0.000 | 0.000 | 0.000 | 0.093 | 0.000 | 0.000 | 2.218 | 2.106 | 2.734 |
| cyclic peptide | | 7430 | 0.064 | 0.000 | 0.000 | 0.000 | 0.000 | 0.000 | 0.072 | 0.000 | 0.000 | 0.000 | 0.000 | 0.000 |
| | | 7431 | 0.062 | 0.000 | 0.000 | 0.000 | 0.000 | 0.000 | 0.073 | 0.000 | 0.000 | 0.000 | 0.000 | 0.000 |
| IAPP ₁₋₁₆ | 10 | 7435 | 0.072 | 0.000 | 0.000 | 0.000 | 1.390 | 0.397 | 0.070 | 0.000 | 0.000 | 0.000 | 0.000 | 1.347 |
| cyclic peptide | | 7436 | 0.067 | 0.000 | 0.000 | 0.000 | 1.252 | 0.000 | 0.077 | 0.000 | 0.000 | 0.000 | 1.460 | 0.000 |
| | | 7437 | 0.087 | 0.000 | 0.000 | 0.000 | 0.000 | 0.000 | 0.081 | 0.000 | 1.546 | 1.829 | 1.738 | 1.745 |

TABLE 6
Immunogenicity Assessment in Guinea Pigs against the CpG and the Th Epitope Portion of
the selected IAPP Peptide Immunogen Constructs

| | | | UBITh1 (SEQ ID NO: 97) | | | | | | CpG ODN | | | | | |
|---|---|---|---|---|---|---|---|---|---|---|---|---|---|---|
| | SEQ ID | Animal | ELISA Log₁₀ Titer | | | | | | ELISA Log10 Titer | | | | | |
| Immunogen | NO: | No | 0 w | 3 w | 6 w | 9 w | 12 w | 15 w | 0 w | 3 w | 6 w | 9 w | 12 w | 15 w |
| UBITh1-εK- | 137 | 7108 | 0.060 | 0.000 | 0.000 | 0.445 | 1.928 | 2.855 | 0.066 | 0.000 | 0.000 | 0.000 | 0.000 | 0.000 |
| KKK-IAPP ₃₀₋₃₇ | | 7109 | 0.086 | 0.000 | 0,000 | 0.000 | 0.000 | 0.854 | 0.085 | 0.000 | 0.000 | 0.000 | 0.000 | 0.000 |
| | | 7110 | 0.075 | 0.000 | 0.000 | 0.559 | 0.281 | 1.934 | 0.089 | 0.000 | 0.000 | 0.000 | 0.000 | 0.000 |
| UBITh1-εK- | 136 | 7517 | 0.054 | 0.000 | 0.000 | 0.000 | 0.000 | 0.000 | 0.052 | 0.000 | 0.000 | 0.000 | 0.000 | 0.000 |
| KKK-IAPP ₂₅₋₃₇ | | 7518 | 0.053 | 0.000 | 0.000 | 0.039 | 1.546 | 1.846 | 0.055 | 0.000 | 0.000 | 0.000 | 0.000 | 0.000 |
| | | 7519 | 0.058 | 0.000 | 1.437 | 1.833 | 1.051 | 1.029 | 0.054 | 1.546 | 1.471 | 1.049 | 1.257 | 1.990 |

TABLE 7
Mapping of IAPP B epitopes with Immune Sera from IAPP Peptide Immunogen Constructs

| | | | A₄₅₀ₙₘ ELISA of Immune Sera from IAPP Peptide Immunogen Constructs | | | | | | | | | | | |
|---|---|---|---|---|---|---|---|---|---|---|---|---|---|---|
| 10 mer peptides | | | SEQ ID NO | | | | | | | | | | | |
| from −7 to 44 of | SEQ | | 118 | 113 | 116 | 123 | 125 | 128 | 129 | 133 | 134 | 125 | 136 | 137 |
| IAPP employed | ID | IAPP | IAPP region | | | | | | | | | | | |
| for epitope mapping | NO | region | (2-7) | (1-9) | (1-16) | (11-20) | (20-29) | (11-37) | (15-37) | (18-37) | (20-37) | (23-37) | (25-37) | (30-37) |
| SRQVEKRRCNTATCAT- | 170 | | | | | | | | | | | | | |
| QRLANFLVRSS- | | | | | | | | | | | | | | |
| NNFGAILSSTNV- | | | | | | | | | | | | | | |
| GSNTYGKRNAVE | | | | | | | | | | | | | | |
| SHQVEKRKCN | 28 | −7-3 | 0.128 | 0.108 | 0.086 | 0.084 | 0.087 | 0.080 | 0.071 | 0.061 | 0.075 | 0.067 | 0.087 | 0.078 |
| HQVEKRKCNT | 29 | −6-4 | 0.126 | 0.097 | 0.101 | 0.104 | 0.093 | 0.077 | 0.070 | 0.057 | 0.075 | 0.068 | 0.081 | 0.079 |
| QVEKRKCNTA | 30 | −5-5 | 0.137 | 0.097 | 0.096 | 0.090 | 0.093 | 0.075 | 0.078 | 0.057 | 0.084 | 0.069 | 0.090 | 0.072 |
| VEKRKCNTAT | 31 | −4-6 | 0.138 | 0.103 | 0.091 | 0.084 | 0.086 | 0.072 | 0.076 | 0.062 | 0.069 | 0.077 | 0.092 | 0.102 |
| EKRKCNTATC | 32 | −3-7 | 0.137 | 0.096 | 0.095 | 0.088 | 0.084 | 0.074 | 0.080 | 0.057 | 0.078 | 0.069 | 0.088 | 0.076 |
| KRKCNTATCA | 33 | −2-8 | 0.154 | 1.063 | 0.125 | 0.090 | 0.081 | 0.075 | 0.080 | 0.062 | 0.082 | 0.081 | 0.089 | 0.082 |
| RKCNTATCAT | 34 | −1-9 | 0.732 | 0.360 | 0.112 | 0.101 | 0.092 | 0.090 | 0.088 | 0.072 | 0.089 | 0.080 | 0.104 | 0.071 |
| KCNTATCATQ | 35 | 1-10 | 1.030 | 0.572 | 0.139 | 0.094 | 0.089 | 0.077 | 0.092 | 0.071 | 0.080 | 0.067 | 0.098 | 0.074 |
| CNTATCATQR | 36 | 2-11 | 0.511 | 0.231 | 0.108 | 0.088 | 0.087 | 0.080 | 0.095 | 0.066 | 0.067 | 0.064 | 0.074 | 0.071 |
| NTATCATQRL | 37 | 3-12 | 0.121 | 0.555 | 0.092 | 0.086 | 0.099 | 0.121 | 0.081 | 0.067 | 0.061 | 0.076 | 0.080 | 0.074 |
| TATCATQRLA | 38 | 4-13 | 0.120 | 0.094 | 0.089 | 0.091 | 0.080 | 0.075 | 0.074 | 0.060 | 0.063 | 0.060 | 0.071 | 0.068 |
| ATCATQRLAN | 39 | 5-14 | 0.124 | 0.094 | 0.092 | 0.093 | 0.092 | 0.069 | 0.077 | 0.059 | 0.063 | 0.062 | 0.078 | 0.076 |
| TCATQRLANF | 40 | 6-15 | 0.123 | 0.099 | 0.096 | 0.230 | 0.097 | 0.081 | 0.078 | 0.061 | 0.064 | 0.061 | 0.067 | 0.067 |
| CATQRLANFL | 41 | 7-16 | 0.118 | 0.095 | 0.093 | 0.110 | 0.088 | 0.075 | 0.085 | 0.060 | 0.072 | 0.060 | 0.070 | 0.066 |
| ATQRLANFLV | 42 | 8-17 | 0.126 | 0.097 | 0.094 | 0.101 | 0.091 | 0.084 | 0.076 | 0.058 | 0.071 | 0.064 | 0.072 | 0.061 |
| TQRLANFLVR | 43 | 9-18 | 0.122 | 0.089 | 0.111 | 0.135 | 0.095 | 0.086 | 0.083 | 0.063 | 0.067 | 0.071 | 0.062 | 0.066 |
| QRLANFLVHS | 44 | 10-19 | 0.120 | 0.111 | 0.108 | 0.150 | 0.105 | 0.097 | 0.096 | 0.088 | 0.126 | 0.092 | 0.101 | 0.093 |
| RLANFLVHSS | 45 | 11-20 | 0.135 | 0.112 | 0.100 | 3.299 | 0.100 | 0.081 | 0.079 | 0.063 | 0.064 | 0.076 | 0.077 | 0.070 |
| LANFLVRSSN | 46 | 12-21 | 0.122 | 0.112 | 0.092 | 0.153 | 0.099 | 0.086 | 0.078 | 0.062 | 0.077 | 0.073 | 0.080 | 0.070 |
| ANFLVHSSNN | 47 | 13-22 | 0.129 | 0.102 | 0.095 | 0.091 | 0.088 | 0.083 | 0.153 | 0.073 | 0.073 | 0.071 | 0.081 | 0.075 |
| NFLVHSSNNF | 48 | 14-23 | 0.125 | 0.103 | 0.096 | 0.091 | 0.085 | 0.082 | 0.087 | 0.069 | 0.064 | 0.129 | 0.071 | 0.091 |
| FLVRSSNNFG | 49 | 15-24 | 0.121 | 0.106 | 0.113 | 0.089 | 0.090 | 0.077 | 0.078 | 0.059 | 0.063 | 0.081 | 0.085 | 0.076 |
| LVRSSNNFGA | 50 | 16-25 | 0.122 | 0.097 | 0.107 | 0.089 | 0.099 | 0.076 | 0.080 | 0.066 | 0.082 | 0.072 | 0.093 | 0.070 |
| VHSSNNFGAI | 51 | 17-26 | 0.134 | 0.103 | 0.101 | 0.089 | 0.084 | 0.079 | 0.078 | 0.068 | 0.058 | 0.068 | 0.077 | 0.070 |
| HSSNNFGAIL | 52 | 18-27 | 0.133 | 0.150 | 0.105 | 0.091 | 1.047 | 0.070 | 0.070 | 3.061 | 0.065 | 0.066 | 0.073 | 0.066 |
| SSNNFGAILS | 53 | 19-28 | 0.135 | 0.102 | 0.105 | 0.101 | 3.550 | 0.083 | 0.082 | 1.953 | 0.081 | 0.086 | 0.076 | 0.065 |
| SHQVEKRKCN- | 170 | | | | | | | | | | | | | |
| TATCATQRLAN- | | | | | | | | | | | | | | |
| FLVHSSNNFGAILSST- | | | | | | | | | | | | | | |
| NVGSNTYGKRNAVE | | | | | | | | | | | | | | |
| SNNFGAILSS | 54 | 20-29 | 0.144 | 0.121 | 0.107 | 0.109 | 1.682 | 0.077 | 0.095 | 1.470 | 0.078 | 0.066 | 0.091 | 0.102 |
| NNFGAILSST | 55 | 21-30 | 0.125 | 0.116 | 0.103 | 0.102 | 0.124 | 0.079 | 0.087 | 0.238 | 0.075 | 0.083 | 0.078 | 0.073 |
| NFGAILSSTN | 56 | 22-31 | 0.113 | 0.095 | 0.090 | 0.100 | 0.130 | 0.079 | 0.077 | 0.571 | 0.063 | 0.081 | 0.407 | 0.076 |
| FGAILSSTNV | 57 | 23-32 | 0.128 | 0.109 | 0.103 | 0.103 | 0.096 | 0.408 | 0.082 | 0.067 | 0.069 | 0.090 | 0.681 | 3.568 |
| GAILSSTNVG | 58 | 24-33 | 0.113 | 0.091 | 0.087 | 0.100 | 0.090 | 0.067 | 0.083 | 0.065 | 0.069 | 0.078 | 4.000 | 0.077 |
| AILSSTNVGS | 59 | 25-34 | 0.125 | 0.090 | 0.088 | 0.088 | 0.081 | 0.073 | 0.072 | 0.083 | 0.069 | 0.085 | 1.329 | 0.085 |
| ILSSTNVGSN | 60 | 26-35 | 0.128 | 0.090 | 0.084 | 0.088 | 0.090 | 0.070 | 0.071 | 0.061 | 0.066 | 0.073 | 0.626 | 0.074 |
| LSSTNVGSNT | 61 | 27-36 | 0.121 | 0.091 | 0.097 | 0.083 | 0.086 | 0.076 | 0.075 | 0.063 | 0.069 | 0.069 | 0.090 | 0.069 |
| SSTNVGSNTY | 62 | 28-37 | 0.107 | 0.086 | 0.082 | 0.089 | 0.089 | 3.937 | 4.000 | 4.000 | 4.000 | 4.000 | 4.000 | 4.000 |
| STNVGSNTYG | 63 | 29-38 | 0.134 | 0.103 | 0.117 | 0.106 | 0.092 | 0.141 | 0.188 | 0.083 | 0.093 | 0.147 | 0.507 | 0.096 |
| TNVGSNTYGK | 64 | 30-39 | 0.137 | 0.117 | 0.109 | 0.117 | 0.119 | 0.177 | 0.374 | 0.119 | 0.147 | 0.317 | 0.566 | 0.186 |
| NVGSNTYGKR | 65 | 31-40 | 0.131 | 0.121 | 0.106 | 0.142 | 0.096 | 0.118 | 0.221 | 0.075 | 0.089 | 0.172 | 0.217 | 0.104 |
| VGSNTYGKRN | 66 | 32-41 | 0.110 | 0.105 | 0.084 | 0.117 | 0.091 | 0.083 | 0.109 | 0.068 | 0.073 | 0.108 | 0.109 | 0.078 |
| GSNTYGKRNA | 67 | 33-42 | 0.124 | 0.111 | 0.095 | 0.102 | 0.084 | 0.103 | 0.162 | 0.084 | 0.080 | 0.110 | 0.130 | 0.085 |
| SNTYGKRNAV | 68 | 34-43 | 0.159 | 0.098 | 0.092 | 0.094 | 0.091 | 0.076 | 0.113 | 0.066 | 0.064 | 0.068 | 0.088 | 0.075 |
| NTYGKRNAVE | 69 | 35-44 | 0.111 | 0.105 | 0.092 | 0.104 | 0.099 | 0.083 | 0.076 | 0.060 | 0.065 | 0.065 | 0.085 | 0.071 |
| KCNTATCATQRLANFL- | 3 | 1-37 | 0.368 | 1.520 | 0.921 | 0.166 | 2.046 | 3.985 | 4.000 | 4.000 | 3.967 | 4.000 | 4.000 | 3.907 |
| VHSSNNF- | | | | | | | | | | | | | | |
| GAILSSTNVGSNTY | | | | | | | | | | | | | | |
| RLANFLVHSSNNF- | 17 | 11-37 | 0.119 | 0.115 | 0.054 | 0.164 | 1.439 | 3.916 | 4.000 | 3.976 | 3.924 | 4.000 | 4.000 | 3.882 |
| GAILSSTNVGSNTY | | | | | | | | | | | | | | |

TABLE 8
Immunogenicity Assessment in Guinea Pigs of IAPP Peptide Immunogen Constructs

| | | | | | | | | Agg. | Agg. | IAPP |
|---|---|---|---|---|---|---|---|---|---|---|
| | | | | | | | | Inhibit. | Inhibit. | Oligomer |
| | | | | | | | | (%) | (%) | Induced |
| SEQ ID | | | | | | | | (mono-> | (oligomer-> | Cytotoxicity |
| NO | Region | Cyclized | Peptide Description | IGN | Monomer | Oligomer | Fibril | fibril) | fibril) | Inhibit. (%) |
| 113 | 1-9 | cyclized | IAPP 1-9-KKK-εK-UBITh1 | 4.82 | + | + | + | 69.99 | 13.62 | 28.86 |
| 114 | 1-9 | cyclized | IAPP 1-9-εK-UBITh1 | 4.83 | + | + | + | 67.29 | 0.00 | 0.00 |
| 115 | 1-13 | cyclized | IAPP 1-13-KKK-εK-UBITh1 | 5.14 | +/− | + | + | 50.24 | 7.64 | 9.42 |
| 116 | 1-16 | cyclized | IAPP 1-16-KKK-εK-UBITh1 | 4.86 | +/− | + | ++ | 91.69 | 29.28 | 0.00 |
| 117 | 2-7 | cyclized | IAPP 2-7-KKK-εK-UBITh1 | 2.53 | − | +/− | − | 54.17 | 26.08 | 32.51 |
| 118 | 2-7 | cyclized | IAPP 2-7-εK-UBITh1 | 3.14 | + | + | +/− | 59.68 | 4.88 | 22.64 |
| 119 | 2-13 | cyclized | IAPP 2-13-KKK-εK-UBITh1 | 4.07 | − | − | + | 81.12 | 0.00 | 36.40 |
| 120 | 2-15 | cyclized | IAPP 2-15-KKK-εK-UBITh1 | 4.20 | − | + | + | 91.04 | 21.49 | 26.26 |
| 121 | 2-15 | cyclized | IAPP 2-15-εK-UBITh1 | 4.53 | − | + | + | 84.66 | 20.35 | 22.75 |
| 122 | 11-20 | | UBITh1-εK-KKK-IAPP 11-20 | 3.10 | +/− | + | − | 41.41 | 13.82 | 0.00 |
| 123 | 11-20 | | IAPP 11-20-KKK-εK-UBITh1 | 3.36 | +/− | + | − | 42.46 | 0.00 | 12.07 |
| 124 | 20-29 | | UBITh1-εK-KKK-IAPP 20-29 | 5.12 | +/− | + | − | 36.39 | 13.85 | 31.20 |
| 125 | 20-29 | | IAPP 20-29-KKK-εK-UBITh1 | 5.18 | +/− | + | − | 57.61 | 26.27 | 34.11 |
| 126 | 10-30 | | IAPP 10-30-KKK-εK-UBITh1 | 4.27 | − | +/− | − | 67.36 | 27.13 | 40.65 |
| 127 | 10-30 | | UBITh1-εK-KKK-IAPP 10-30 | BDL | − | +/− | − | 35.53 | 38.62 | 20.78 |
| 128 | 11-37 | | UBITh1-εK-KKK-IAPP 11-37 | 6.84 | ++ | +++ | + | 86.31 | 37.48 | 78.99 |
| 129 | 15-37 | | UBITh1-εK-KKK-IAPP 15-37 | 10.00 | ++ | +++ | + | 80.60 | 51.39 | 91.35 |
| 130 | 18-30 | | UBITh1-εK-KKK-IAPP 18-30 | 5.14 | + | +++ | − | 16.00 | — | 0.00 |
| 131 | 18-33 | | UBITh1-εK-KKK-IAPP 18-33 | 5.67 | ++ | +++ | + | 0.00 | — | 0.00 |
| 132 | 18-35 | | UBITh1-εK-KKK-IAPP 18-35 | 5.01 | +/− | ++ | − | 0.00 | — | 0.00 |
| 133 | 18-37 | | UBITh1-εK-KKK-IAPP 18-37 | 6.33 | +/− | + | + | 89.55 | 53.21 | 99.20 |
| 134 | 20-37 | | UBITh1-εK-KKK-IAPP 20-37 | 5.37 | + | +/− | + | 84.01 | 46.44 | 97.57 |
| 135 | 23-37 | | UBITh1-εK-KKK-IAPP 23-37 | 7.17 | ++ | ++ | + | 79.82 | 62.06 | 83.47 |
| 136 | 25-37 | | UBITh1-εK-KKK-IAPP 25-37 | 8.55 | ++ | ++ | + | 79.87 | 59.44 | 92.46 |
| 137 | 30-37 | | UBITh1-εK-KKK-IAPP 30-37 | 10.00 | + | + | + | 29.12 | 8.01 | 17.41 |
| 138 | 30-37 | | UBITh1-εK-IAPP 30-37 | 5.81 | + | + | + | 48.05 | 49.42 | 0.00 |

1. An IAPP peptide immunogen construct having about 20 or more amino acids, represented by the formulae:
(Th)ₘ-(A)ₙ-(IAPP functional B cell epitope peptide)-X
or
(IAPP functional B cell epitope peptide)-(A)ₙ-(Th)ₘ-X
or
(Th)ₘ-(A)ₙ-(IAPP functional B cell epitope peptide)-(A)ₙ-(Th)ₘ-X
wherein
Th is a heterologous T helper epitope;
A is a heterologous spacer;
(IAPP functional B cell epitope peptide) is a B cell epitope peptide having from 6 to about 28 amino acid residues of IAPP (SEQ ID NO: 3);
X is an α-COOH or α-CONH₂ of an amino acid;
m is from 1 to about 4; and
n is from 0 to about 10.

2. The IAPP peptide immunogen construct according to claim 1, wherein the IAPP functional B cell epitope peptide is selected from the group consisting of SEQ ID NOs: 8-69.

3. The IAPP peptide immunogen construct according to claim 1, wherein the Th epitope is selected from the group consisting of SEQ ID NOs: 73-112 and 171-182.

4. The IAPP peptide immunogen construct according to claim 1, wherein the IAPP functional B cell epitope peptide is selected from the group consisting of SEQ ID NOs: 8-26 and the Th epitope is selected from the group consisting of SEQ ID NOs: 73-112 and 171-182.

5. The IAPP peptide immunogen construct according to claim 1, wherein the peptide immunogen construct is selected from the group consisting of SEQ ID NOs: 113-167.

6. An IAPP peptide immunogen construct comprising:
a. a B cell epitope comprising from about 6 to about 28 amino acid residues from the IAPP sequence of SEQ ID NOs: 3-7;
b. a T helper epitope comprising an amino acid sequence selected from the group consisting of SEQ ID NOs: 73-112 and 171-182, and any combination thereof; and
c. an optional heterologous spacer selected from the group consisting of an amino acid, Lys-, Gly-, Lys-Lys-Lys-, (α, ε-N)Lys, ε-N-Lys-Lys-Lys-Lys (SEQ ID NO: 71), Lys-Lys-Lys-ε-N-Lys (SEQ ID NO: 72), and Pro-Pro-Xaa-Pro-Xaa-Pro (SEQ ID NO: 70), and any combination thereof,
wherein the B cell epitope is covalently linked to the T helper epitope directly or through the optional heterologous spacer.

7. The IAPP peptide immunogen construct of claim 6, wherein the B cell epitope is selected from the group consisting of SEQ ID NOs: 8-69.

8. The IAPP peptide immunogen construct of claim 6, wherein the optional heterologous spacer is (α, ε-N)Lys, ε-N-Lys-Lys-Lys-Lys (SEQ ID NO: 71), Lys-Lys-Lys-ε-N-Lys (SEQ ID NO: 72), or Pro-Pro-Xaa-Pro-Xaa-Pro (SEQ ID NO: 70), where Xaa is any amino acid.

9. The IAPP peptide immunogen construct of claim 6, wherein the T helper epitope is covalently linked to the amino- or carboxyl-terminus of the B cell epitope.

10. The IAPP peptide immunogen construct of claim 6, wherein the T helper epitope is covalently linked to the amino- or carboxyl-of the B cell epitope through the optional heterologous spacer.

11. A composition comprising the IAPP peptide immunogen construct according to claim 1.

12. A pharmaceutical composition comprising:
a. a peptide immunogen construct according to claim 1; and
b. a pharmaceutically acceptable delivery vehicle and/or adjuvant.

13. The pharmaceutical composition of claim 12, wherein
a. the IAPP functional B cell epitope peptide is selected from the group consisting of SEQ ID NOs: 8-69;
b. the Th epitope is selected from the group consisting of SEQ ID NOs: 73-112 and 171-182; and
c. the heterologous spacer is selected from the group consisting of an amino acid, Lys-, Gly-, Lys-Lys-Lys-, (α, ε-N)Lys, ε-N-Lys-Lys-Lys-Lys (SEQ ID NO: 71), Lys-Lys-Lys-ε-N-Lys (SEQ ID NO: 72), and Pro-Pro-Xaa-Pro-Xaa-Pro (SEQ ID NO: 70), and any combination thereof; and
wherein the IAPP peptide immunogen construct is mixed with an CpG oligodeoxynucleotide (ODN) to form a stabilized immunostimulatory complex.

14. The pharmaceutical composition of claim 12, wherein
a. the IAPP peptide immunogen construct is selected from the group consisting of SEQ ID NOs: 113-139 and 140-167; and
wherein the IAPP peptide immunogen construct is mixed with an CpG oligodeoxynucleotide (ODN) to form a stabilized immunostimulatory complex.

15. A method for generating antibodies against IAPP in an animal comprising administering the pharmaceutical composition according to claim 12 to the animal.

16. An isolated antibody or epitope-binding fragment thereof that specifically binds to the amino acid sequence of SEQ ID NOs: 8-25.

17. The isolated antibody or epitope-binding fragment thereof according to claim 16 bound to the IAPP peptide immunogen construct.

18. A composition comprising the isolated antibody or epitope-binding fragment thereof according to claim 16.

19. A method of preventing and/or treating disorders associated with aggregated IAPP. in an animal comprising administering the pharmaceutical composition of claim 12 to the animal.
